SEQUENCE LISTING (15 sequences)

SEQ ID NO: 1 - protein, 1477 aa, Unknown
  Unknown Streptococcus species
MDETQDKTVTQSNSGTTASLVTSPEATKEADKRTNTKEADVLTPAKETNAVETATTTNTQATAEAATTATTADVAVAAVPNKEAVVTTDAPAVTTEKAEEQPATVKAEVVNTEVKAPEAALKDSEVEAALSLKNIKNIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYRTNEVYTSVRYGKDIMTANDTEGSKYSRTSGQVTLVANNPKLNLDQSAKLNVEMGKIHANQKYRALIVGTADGIKNFTSDADAIAAGYVKETDSNGVLTFGANDIKGYETFDMSGFVAVWVPVGASDNQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANSKSSGKDYQAKYGGEFLAELKAKYPEMFKVNMISTGKPIDDSVKLKQWKAEYFNGTNVLERGVGYVLSDEATGKYFTVTKEGNFIPLQLTGKEKVITGFSSDGKGITYFGTSGTQAKSAFVTFNGNTYYFDARGHMVTNSEYSPNGKDVYRFLPNGIMLSNAFYIDANGNTYLYNSKGQMYKGGYTKFDVSETDKDGKESKVVKFRYFTNEGVMAKGVTVIDGFTQYFGEDGFQAKDKLVTFKGKTYYFDAHTGNGIKDTWRNINGKWYYFDANGVAATGAQVINGQKLYFNEDGSQVKGGVVKNADGTYSKYKEGFGELVTNEFFTTDGNVWYYAGANGKTVTGAQVINGQHLYFNADGSQVKGGVVKNADGTYSKYNASTGERLTNEFFTTGDNNWYYIGANGKSVTGEVKIGDDTYFFAKDGKQVKGQTVSAGNGRISYYYGDSGKRAVSTWIEIQPGVYVYFDKNGLAYPPRVLN

SEQ ID NO: 2 - protein, 1341 aa, Unknown
  Unknown Streptococcus species
MIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEDWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVYIRAHDNNVQDIIAEIIKKEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYSTNEVYTSVRYGKDIMTANDTEGSKYSRTSGQVTLVANNPKLTLDQSAKLNVEMGKIHANQKYRALIVGTADGIKNFTSDADAIAAGYVKETDSNGVLTFGANDIKGYETFDMSGFVAVWVPVGASDDQDIRVAPSTEAKKEGELTLKATEAYDSQLIYEGFSNFQTIPDGSDPSVYTNRKIAENVDLFKSWGVTSFEMAPQFVSADGGTFLDSVIQNGYAFADRYDLAMSKNNKYGSKEDLRDALKALHKAGIQAIADWVPDQIYQLPGKEVVTATRTDGAGRKIADAIIDHSLYVANTKSSGKDYQAKYGGEFLAELKAKYPEMFKVNMISTGKPIDDSVKLKQWKAEYFNGTNVLERGVGYVLSDEATGKYFTVTKDGNFIPLQLTGNEKVVTGFSNDGKGITYFGTSGTQAKSAFVTFNGNTYYFDARGHMVTNGEYSPNGKDVYRFLPNGIMLSNAFYVDANGNTYLYNSKGQMYKGGYTKFDVTETDKDGKESKVVKFRYFTNEGVMAKGVTVIDGFTQYFGEDGFQAKDKLVTFKGKTYYFDAHTGNAIKDTWRNINGKWYHFDANGVAATGAQVINGQKLYFNEDGSQVKGGVVKNADGTYSKYKEGSGELVTNEFFTTDGNVWYYAGANGKTVTGAQVINGQHLYFNADGSQVKGGVVKNADGTYSKYDASTGERLTNEFFTTGDNNWYYIGANGKSVTGEVKIGDDTYFFAKDGKQVKGQTVSAGNGRISYYYGDSGKRAVSTWIEIQPGVYVYFDKNGIAYPPRVLN

SEQ ID NO: 3 - protein, 1435 aa, Streptococcus sobrinus
MVDGKYYYYDQDGNVKKNFAVSVGDKIYYFDETGAYKDTSKVDADKSSSAVSQNATIFAANNRAYSTSAKNFEAVDNYLTADSWYRPKSILKDGKTWTESGKDDFRPLLMAWWPDTETKRNYVNYMNKVVGIDKTYTAETSQADLTAAAELVQARIEQKITSENNTKWLREAISAFVKTQPQWNGESEKPYDDHLQNGALLFDNQTDLTPDTQSNYRLLNRTPTNQTGSLDSRFTYNPNDPLGGYDFLLANDVDNSNPVVQAEQLNWLHYLLNFGSIYANDADANFDSIRVDAVDNVDADLLQISSDYLKAAYGIDKNNKNANNHVSIVEAWSDNDTPYLHDDGDNLMNMDNKFRLSMLWSLAKPLDKRSGLNPLIHNSLVDREVDDREVETVPSYSFARAHDSEVQDIIRDIIKAEINPNSFGYSFTQEEIEQAFKIYNEDLKKTDKKYTHYNVPLSYTLLLTNKGSIPRVYYGDMFTDDGQYMANKTVNYDAIESLLKARMKYVSGGQAMQNYQIGNGEILTSVRYGKGALKQSDKGDATTRTSGVGVVMGNQPNFSLDGKVVALNMGAAHANQEYRALMVSTKDGVATYATDADASKAGLVKRTDENGYLYFLNDDLKGVANPQVSGFLQVWVPVGAADDQDIRVAASDTASTDGKSLHQDAAMDSRVMFEGFSNFQSFATKEEEYTNVVIANNVDKFVSWGITDFEMAPQYVSSTDGQFLDSVIQNGYAFTDRYDLGMSKANKYGTADQLVKAIKALHAKGLKVMADWVPDQMYTFPKQEVVTVTRTDKFGKPIAGSQINHSLYVTDTKSSGDDYQAKYGGAFLDELKEKYPELFTKKQISTGQAIDPSVKIKQWSAKYFNGSNILGRGADYVLSDQVSNKYFNVASDTLFLPSSLLGKVVESGIRYDGKGYIYNSSATGDQVKASFITEAGNLYYFGKDGYMVTGAQTINGANYFFLENGTALRNTIYTDAQGNSHYYANDGKRYENGYQQFGNDWRYFKDGNMAVGLTTVDGNVQYFDKDGVQAKDKIIVTRDGKVRYFDQHNGNAATNTFIADKTGHWYYLGKDGVAVTGAQTVGKQKLYFEANGQQVKGDFVTSDEGKLYFYDVDSGDMWTDTFIEDKAGNWFYLGKDGAAVTGAQTIRGQKLYFKANGQQVKGDIVKGTDGKIRYYDAKSGEQVFNKTVKAADGKTYVIGNDGVAVDPSVVKGQTFKDASGALRFYNLKGQLVTGSGWYETANHDWVYIQSGKALTGEQTINGQHLYFKEDGHQVKGQLVTGTDGKVRYYDANSGDQAFNKSVTVNGKTYYFGNDGTAQTAGNPKGQTFKDGSDIRFYSMEGQLVTGSGWYENAQGQWLYVKNGKVLTGLQTVGSQRVYFDENGIQAKGKAVRTSDGKIRYFDENSGSMITNQWKFVYGQYYYFGNDGARIYRGWN

SEQ ID NO: 4 - protein, 1341 aa, Streptococcus salivarius [sequence omitted]

SEQ ID NO: 5 - protein, 1247 aa, Streptococcus salivarius
MPSHIKTINGKQYYVEDDGTIRKNYVLERIGGSQYFNAETGELSNQKEYRFDKNGGTGSSADSTNTNVTVNGDKNAFYGTTDKDIELVDGYFTANTWYRPKEILKDGKEWTASTENDKRPLLTVWWPSKAIQASYLNYMKEQGLGTNQTYTSFSSQTQMDQAALEVQKRIEERIAREGNTDWLRTTIKNFVKTQPGWNSTSENLDNNDHLQGGALLYNNDSRTSHANSDYRLLNRTPTSQTGKHNPKYTKDTSNGGFEFLLANDIDNSNPAVQAEQLNWLHYIMNIGTITGGSEDENFDGVRVDAVDNVNADLLQIASDYFKAKYGADQSQDQAIKHLSILEAWSHNDAYYNEDTKGAQLPMDDPMHLALVYSLLRPIGNRSGVEPLISNSLNDRSESGKNSKRMANYAFVRAHDSEVQSIIGQIIKNEINPQSTGNTFTLDEMKKAFEIYNKDMRSANKQYTQYNIPSAYALMLTHKDTVPRVYYGDMYTDDGQYMAQKSPYYDAIETLLKGRIRYAAGGQDMKVNYIGYGNTNGWDAAGVLTSVRYGTGANSASDTGTAETRNQGMAVIVSNQPALRLTSNLTINMGAAHRNQAYRPLLLTTNDGVATYLNDSDANGIVKYTDGNGNLTFSANEIRGIRNPQVDGYLAVWVPVGASENQDVRVAPSKEKNSSGLVYESNAALDSQVIYEGFSNFQDFVQNPSQYTNKKIAENANLFKSWGITSFEFAPQYVSSDDGSFLDSVIQNGYAFTDRYDIGMSKDNKYGSLADLKAALKSLHAVGISAIADWVPDQIYNLPGDEVVTATRVNNYGETKDGAIIDHSLYAAKTRTFGNDYQGKYGGAFLDELKRLYPQIFDRVQISTGKRMTTDEKITQWSAKYMNGTNILDRGSEYVLKNGLNGYYGTNGGKVSLPKVVGSNQSTNGDNQNGDGSGKFEKRLFSVRYRYNNGQYAKNAFIKDNDGNVYYFDNSGRMAVGEKTIDGKQYFFLANGVQLRDGYRQNRRGQVFYYDQNGVLNANGKQDPKPDNNNNASGRNQFVQIGNNVWAYYDGNGKRVTGHQNINGQELFFDNNGVQVKGRTVNENGAIRYYDANSGEMARNRFAEIEPGVWAYFNNDGTAVKGSQNINGQDLYFDQNGRQVKGALANVDGNLRYYDVNSGELYRNRFHEIDGSWYYFDGNGNAVKGMVNINGQNLLFDNNGKQIKGHLVRVNGVVRYFDPNSGEMAVNRWVEVSPGWWVYFDGEGRGQI

SEQ ID NO: 6 - protein, 1477 aa, Streptococcus salivarius; identical to SEQ ID NO: 1

SEQ ID NO: 7 - protein, 1436 aa, Streptococcus downei
MVDGKYYYYDQDGNVKKNFAVSVGEKIYYFDETGAYKDTSKVEADKSGSDISKEETTFAANNRAYSTSAENFEAIDNYLTADSWYRPKSILKDGKTWTESSKDDFRPLLMAWWPDTETKRNYVNYMNKVVGIDKTYTAETSQADLTAAAELVQARIEQKITTEQNTKWLREAISAFVKTQPQWNGESEKPYDDHLQNGALKFDNQSDLTPDTQSNYRLLNRTPTNQTGSLDSRFTYNANDPLGGYELLLANDVDNSNPIVQAEQLNWLHYLLNFGTIYAKDADANFDSIRVDAVDNVDADLLQISSDYLKAAYGIDKNNKNANNHVSIVEAWSDNDTPYLHDDGDNLMNMDNKFRLSMLWSLAKPLDKRSGLNPLIHNSLVDREVDDREVETVPSYSFARAHDSEVQDLIRDIIKAEINPNAFGYSFTQDEIDQAFKIYNEDLKKTDKKYTHYNVPLSYTLLLTNKGSIPRVYYGDMFTDDGQYMANKTVNYDAIESLLKARMKYVAGGQAMQNYQIGNGEILTSVRYGKGALKQSDKGDATTRTSGVGVVMGNQPNFSLDGKVVALNMGAAHANQEYRALMVSTKDGVATYATDADASKAGLVKRTDENGYLYFLNDDLKGVANPQVSGFLQVWVPVGAADDQDIRVAASDTASTDGKSLHQDAAMDSRVMFEGFSNFQSFATKEEEYTNVVIANNVDKFVSWGITDFEMAPQYVSSTDGQFLDSVIQNGYAFTDRYDLGMSKANKYGTADQLVKAIKALHAKGLKVMADWVPDQMYTFPKQEVVTVTRTDKFGKPIAGSQINHSLYVTDTKSSGDDYQAKYGGAFLDELKEKYPELFTKKQISTGQAIDPSVKIKQWSAKYFNGSNILGRGADYVLSDQASNKYLNVSDDKLFLPKTLLGQVVESGIRFDGTGYVYNSSTTGEKVTDSFITEAGNLYYFGQDGYMVTGAQNIKGSNYYFLANGAALRNTVYTDAQGQNHYYGNDGKRYENGYQQFGNDSWRYFKNGVMALGLTTVDGHVQYFDKDGVQAKDKIIVTRDGKVRYFDQHNGNAVTNTFVADKTGHWYYLGKDGVAVTGAQTVGKQHLYFEANGQQVKGDFVTAKDGKLYFYDVDSGDMWTNTFIEDKAGNWFYLGKDGAAVTGAQTIKGQKLYFKANGQQVKGDIVKDADGKIRYYDAQTGEQVFNKSVSVNGKTYYFGSDGTAQTQANPKGQTFKDGSGVLRFYNLEGQYVSGSGWYETAEHEWVYVKSGKVLTGAQTIGNQRVYFKDNGHQVKGQLVTGNDGKLRYYDANSGDQAFNKSVTVNGKTYYFGSDGTAQTQANPKGQTFKDGSGVLRFYNLEGQYVSGSGWYKNAQGQWLYVKDGKVLTGLQTVGNQKVYFDKNGIQAKGKAVRTSDGKVRYFDENSGSMITNQWKFVYGQYYYFGSDGAAVYRGWN

SEQ ID NO: 8 - protein, 1313 aa, Streptococcus mutans
MIDGKYYYYDNNGKVRTNFTLIADGKILHFDETGAYTDTSIDTVNKDIVTTRSNLYKKYNQVYDRSAQSFEHVDHYLTAESWYRPKYILKDGKTWTQSTEKDFRPLLMTWWPSQETQRQYVNFMNAQLGINKTYDDTSNQLQLNIAAATIQAKIEAKITTLKNTDWLRQTISAFVKTQSAWNSDSEKPFDDHLQNGAVLYDNEGKLTPYANSNYRILNRTPTNQTGKKDPRYTADNTIGGYEFLLANDVDNSNPVVQAEQLNWLHFLMNFGNIYANDPDANFDSIRVDAVDNVDADLLQIAGDYLKAAKGIHKNDKAANDHLSILEAWSDNDTPYLHDDGDNMINMDNKLRLSLLFSLAKPLNQRSGMNPLITNSLVNRTDDNAETAAVPSYSFIRAHDSEVQDLIRDIIKAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKAMDTGDRTTRTSGVAVIEGNNPSLRLKASDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYALPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFNISDNKEINFLPKTLLNQDSQVGFSYDGKGYVYYSTSGYQAKNTFISEGDKWYYFDNNGYMVTGAQSINGVNYYFLPNGLQLRDAILKNEDGTYAYYGNDGRRYENGYYQFMSGVWRHFNNGEMSVGLTVIDGQVQYFDEMGYQAKGKFVTTADGKIRYFDKQSGNMYRNRFIENEEGKWLYLGEDGAAVTGSQTINGQHLYFRANGVQVKGEFVTDRHGRISYYDGNSGDQIRNRFVRNAQGQWFYFDNNGYAVTGARTINGQHLYFRANGVQVKGEFVTDRHGRISYYDGNSGDQIRNRFVRNAQGQWFYFDNNGYAVTGARTINGQHLYFRANGVQVKGEFVTDRYGRISYYDGNSGDQIRNRFVRNAQGQWFYFDNNGYAVTGARTINGQHLYFRANGVQVKGEFVTDRYGRISYYDANSGERVRIN

SEQ ID NO: 9 - protein, 1323 aa, Streptococcus dentirousetti
MVDGKYYYYDADGNVKKNFAVSVGDAIFYFDETGAYKDTSKVDADKTSSSVNQTTETFAANNRAYSTAAENFEAIDNYLTADSWYRPKSILKDGTTWTESTKDDFRPLLMAWWPDTETKRNYVNYMNKVVGIDKTYTAETSQADLTAAAELVQARIEQKITSEKNTKWLREAISAFVKTQPQWNGESEKPYDDHLQNGALKFDNETSLTPDTQSGYRILNRTPTNQTGSLDPRFTFNQNDPLGGYEYLLANDVDNSNPVVQAESLNWLHYLLNFGSIYANDPEANFDSIRVDAVDNVDADLLQISSDYLKSAYKIDKNNKNANDHVSIVEAWSDNDTPYLNDDGDNLMNMDNKFRLSMLWSLAKPTNVRSGLNPLIHNSVVDREVDDREVEATPNYSFARAHDSEVQDLIRDIIKAEINPNSFGYSFTQEEIDQAFKIYNEDLKKTNKKYTHYNVPLSYTLLLTNKGSIPRIYYGDMFTDDGQYMANKTVNYDAIESLLKARMKYVSGGQAMQNYNIGNGEILTSVRYGKGALKQSDKGDKTTRTSGIGVVMGNQSNFSLEGKVVALNMGATHTKQKYRALMVSTETGVAIYNSDEEAEAAGLIKTTDENGYLYFLNDDLKGVANPQVSGFLQVWVPVGAPADQDIRVAATDAASTDGKSLHQDAALDSRVMFEGFSNFQSFATKEEEYTNVVIAKNVDKFVSWGITDFEMAPQYVSSTDGTFLDSVIQNGYAFTDRYDLGMSKANKYGTADQLVAAIKALHAKGLRVMADWVPDQMYTFPKKEVVTVTRTDKFGNPVAGSQINHTLYVTDTKGSGDDYQAKYGGAFLDELKEKYPELFTKKQISTGQAIDPSVKIKQWSAKYFNGSNILGRGANYVLSDQASNKYFNVAEGKVFLPAAMLGKVVESGIRFDGKGYIYNSSTTGEQVKDSFITEAGNLYYFGKDGYMVMGAQNIQGANYYFLANGAALRNSILTDQDGKSHYYANDGKRYENGYYQFGNDSWRYFENGVMAVGLTRVAGHDQYFDKDGIQAKNKIIVTRDGKVRYFDEHNGNAATNTFISDQAGHWYYLGKDGVAVTGAQTVGKQHLYFEANGQQVKGDFVTAKDGKLYFLDGDSGDMWTDTFVQDKAGHWFYLGKDGAAVTGAQTVRGQKLYFKANGQQVKGDIVKGADGKIRYYDANSGDQVYNRTVKGSDGKTYIIGNDGVAITQTIAKGQTIKDGSVLRFYSMEGQLVTGSGWYSNAKGQWLYVKNGQVLTGLQTVGSQRVYFDANGIQAKGKAVRTSDGKLRYFDANSGSMITNQWKEVNGQYYYFDNNGVAIYRGWN

SEQ ID NO: 10 - protein, 1348 aa, Streptococcus oralis
MIDGKNYYVQDDGTVKKNFAVELNGRILYFDAETGALVDSNEYQFQQGTSSLNNEFSQKNAFYGTTDKDIETVDGYLTADSWYRPKFILKDGKTWTASTETDLRPLLMAWWPDKRTQINYLNYMNQQGLGAGAFENKVEQALLTGASQQVQRKIEEKIGKEGDTKWLRTLMGAFVKTQPNWNIKTESETTGTKKDHLQGGALLYTNNEKSPHADSKFRLLNRTPTSQTGTPKYFIDKSNGGYEFLLANDFDNSNPAVQAEQLNWLHYMMNFGSIVANDPTANFDGVRVDAVDNVNADLLQIASDYFKSRYKVGESEEEAIKHLSILEAWSDNDPDYNKDTKGAQLAIDNKLRLSLLYSFMRNLSIRSGVEPTITNSLNDRSSEKKNGERMANYIFVRAHDSEVQTVIADIIRENINPNTDGLTFTMDELKQAFKIYNEDMRKADKKYTQFNIPTAHALMLSNKDSITRVYYGDLYTDDGQYMEKKSPYHDAIDALLRARIKYVAGGQDMKVTYMGVPREADKWSYNGILTSVRYGTGANEATDEGTAETRTQGMAVIASNNPNLKLNEWDKLQVNMGAAHKNQYYRPVLLTTKDGISRYLTDEEVPQSLWKKTDANGILTFDMNDIAGYSNVQVSGYLAVWVPVGAKADQDARTTASKKKNASGQVYESSAALDSQLIYEGFSNFQDFATRDDQYTNKVIAKNVNLFKEWGVTSFELPPQYVSSQDGTFLDSIIQNGYAFEDRYDMAMSKNNKYGSLKDLLNALRALHSVNIQAIADWVPDQIYNLPGKEVVTATRVNNYGTYREGAEIKEKLYVANSKTNETDFQGKYGGAFLDELKAKYPEIFERVQISNGQKMTTDEKITKWSAKYFNGTNILGRGAYYVLKDWASNDYLTNRNGEIVLPKQLVNKNSYTGFVSDANGTKFYSTSGYQAKNSFIQDENGNWYYFDKRGYLVTGAHEIDGKHVYFLKNGIQLRDSIREDENGNQYYYDQTGAQVLNRYYTTDGQNWRYFDAKGVMARGLVKIGDGQQFFDENGYQVKGKIVSAKDGKLRYFDKDSGNAVINRFAQGDNPSDWYYFGVEFAKLTGLQKIGQQTLYFDQDGKQVKGKIVTLSDKSIRYFDANSGEMAVGKFAEGAKNEWYYFDKTGKAVTGLQKIGKQTLYFDQDGKQVKGKVVTLADKSIRYFDADSGEMAVGKFAEGAKNEWYYFDQTGKAVTGLQKIDKQTLYFDQDGKQVKGKIVTLSDKSIRYFDANSGEMATNKFVEGSQNEWYYFDQAGKAVTGLQQVGQQTLYFTQDGKQVKGKVVDVNGVSRYFDANSGDMARSKWIQLEDGSWMYFDRDGRGQNFGRN

SEQ ID NO: 11 - protein, 1348 aa, Streptococcus sanguinis
MIDGKKYYVQDDGTVKKNFAVELNGKILYFDAETGALIDSAEYQFQQGTSSLNNEFTQKNAFYGTTDKDVETIDGYLTADSWYRPKFILKDGKTWTASTEIDLRPLLMAWWPDKQTQVSYLNYMNQQGLGAGAFENKVEQAILTGASQQVQRKIEERIGKEGDTKWLRTLMGAFVKTQPNWNIKTESETTGTNKDHLQGGALLYSNSDKTSHANSKYRILNRTPTNQTGTPKYFIDKSNGGYEFLLANDFDNSNPAVQAEQLNWLHFMMNFGSIVANDPTANFDGVRVDAVDNVNADLLQIASDYFKSRYKVGESEEEAIKHLSILEAWSDNDPDYNKDTKGAQLPIDNKLRLSLLYSFMRKLSIRSGVEPTITNSLNDRSTEKKNGERMANYIFVRAHDSEVQTVIADIIRENINPNTDGLTFTMDELKQAFKIYNEDMRKADKKYTQFNIPTAHALMLSNKDSITRVYYGDLYTDDGQYMEKKSPYHDAIDALLRARIKYVAGGQDMKVTYMGVPREADKWSYNGILTSVRYGTGANEATDEGTAETRTQGMAVIASNNPNLKLNEWDKLQVNMGAAHKNQYYRPVLLTTKDGISRYLTDEEVPQSLWKKTDANGILTFDMNDIAGYSNVQVSGYLAVWVPVGAKADQDARVTASKKKNASGQVYESSAALDSQLIYEGFSNFQDFATRDDQYTNKVIAKNVNLFKEWGVTSFELPPQYVSSQDGTFLDSIIQNGYAFEDRYDMAMSKNNKYGSLNDLLNALRALHSVNIQAIADWVPDQIYNLPGKEVVTATRVNNYGTYREGSEIKENLYVANTKTNGTDYQGKYGGAFLDELKAKYPEIFERVQISNGQKMTTDEKITKWSAKHFNGTNILGRGAYYVLKDWASNEYLNNKNGEMVLPKQLVNKNAYTGFVSDASGTKYYSTSGYQARNSFIQDENGNWYYFNNRGYLVTGAQEIDGKQLYFLKNGIQLRDSLREDENGNQYYYDKTGAQVLNRYYTTDGQNWRYFDVKGVMARGLVTMGGNQQFFDQNGYQVKGKIARAKDGKLRYFDKDSGNAAANRFAQGDNPSDWYYFGADGVAVTGLQKVGQQTLYFDQDGKQVKGKVVTLADKSIRYFDANSGEMAVNKFVEGAKNVWYYFDQAGKAVTGLQTINKQVLYFDQDGKQVKGKVVTLADKSIRYFDANSGEMAVGKFAEGAKNEWYYFDQAGKAVTGLQKIGQQTLYFDQNGKQVKGKVVTLADKSIRYFDANSGEMASNKFVEGAKNEWYYFDQAGKAVTGLQQIGQQTLYFDQNGKQVKGKIVYVNGANRYFDANSGEMARNKWIQLEDGSWMYFDRNGRGRRFGWN

SEQ ID NO: 12 - protein, 1340 aa, Unknown
  Unknown Streptococcus species
MIDGKYYYVNEDGSHKENFAITVNGQLLYFGKDGALTSSSTYSFTQGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSNYRLLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDVAALAMENKQRLALLFSLAKPIKERTPAVSPLYNNTFNTTQRDEKTDWINKDGSKAYNEDGTVKKSTIGKYNEKYGDASGNYVFIRAHDNNVQDIIAEIIKKEINEKSDGFTITDSEMKRAFEIYNKDMLSNDKKYTLNNIPAAYAVMLQNMETITRVYYGDLYTDDGNYMEAKSPYYDTIVNLMKSRIKYVSGGQAQRSYWLPTDGKMDKSDVELYRTNEVYTSVRYGKDIMTADDTQGSKYSRTSGQVTLVVNNPKLTLDQSAKLNVVMGKIHANQKYRALIVGTPNGIKNFTSDAEAIAAGYVKETDGNGVLTFGANDIKGYETFDMSGFVAVWVPVGASDDQDIRVAASTAAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRNALKALHKAGIQAIADWVPDQIYQLPGKEVVTATRTDGAGRKISDAIIDHSLYVANSKSSGKDYQAKYGGEFLAELKAKYPEMFKVNMISTGKPIDDSVKLKQWKAEYFNGTNVLDRGVGYVLSDEATGKYFTVTKEGNFIPLQLKGNKKVITGFSSDGKGITYFGTSGNQAKSAFVTFNGNTYYFDARGHMVTNGEYSPNGKDVYRFLPNGIMLSNAFYVDGNGNTYLYNSKGQMYKGGYSKFDVTETKDGKESKVVKFRYFTNEGVMAKGVTVVDGFTQYFNEDGIQSKDELVTYNGKTYYFEAHTGNAIKNTWRNIKGKWYHFDANGVAATGAQVINGQHLYFNEDGSQVKGSIVKNADGTFSKYKDSSGDLVVNEFFTTGDNVWYYAGANGKTVTGAQVINGQHLFFKEDGSQVKGDFVKNSDGTYSKYDAASGERLTNEFFTTGDNHWYYIGANGKTVTGEVKIGDDTYFFAKDGKQLKGQIVTTRSGRISYYFGDSGKKAISTWVEIQPGVFVFFDKNGLAYPPENMN

SEQ ID NO: 13 - protein, 1340 aa, Streptococcus salivarius
MIDGKYYYVNKDGSHKENFAITVNGQLLYFGKDGALTSSSTYSFTQGTTNIVDGFSKNNRAYDSSEASFELIDGYLTADSWYRPVSIIKDGVTWQASTKEDFRPLLMAWWPNVDTQVNYLNYMSKVFNLDAKYTSTDKQVDLNRAAKDIQVKIEQKIQAEKSTQWLREAISAFVKTQPQWNKETENFSKGGGEDHLQGGALLYVNDPRTPWANSNYRLLNRTATNQTGTIDKSVLDEQSDPNHMGGFDFLLANDVDTSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKKAFEIYNKDMLSSDKKYTLNNIPAAYAVMLQNMETITRVYYGDLYTDDGHYMETKSPYYDTIVNLMKNRIKYVSGGQAQRSYWLPTDGKMDKSDVELYRTNEVYTSVRYGKDIMTADDTQGSKYSRTSGQVTLVVNNPKLSLDKSAKLDVEMGKIHANQKYRALIVGTPNGIKNFTSDAEAIAAGYVKETDGNGVLTFGANDIKGYETFDMSGFVAVWVPVGASDDQDIRVAASTAAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRNALKALHKAGIQAIADWVPDQIYQLPGKEVVTATRTDGAGRKISDAIIDHSLYVANSKSSGKDYQAKYGGEFLAELKAKYPEMFKVNMISTGKPIDDSVKLKQWKAEYFNGTNVLDRGVGYVLSDEATGKYFTVTKEGNFIPLQLKGNEKVITGFSSDGKGITYFGTSGNQAKSAFVTFNGNTYYFDARGHMVTNGEYSPNGKDVYRFLPNGIMLSNAFYVDGNGNTYLYNSKGQMYKGGYSKFDVTETKDGKESKVVKFRYFTNEGVMAKGVTVVDGFTQYFNEDGIQSKDELVTYNGKTYYFEAHTGNAIKNTWRNIKGKWYHFDANGVAATGAQVINGQHLYFNEDGSQVKGGVVKNADGTFSKYKDGSGDLVVNEFFTTGDNVWYYAGANGKTVTGAQVINGQHLFFKEDGSQVKGDFVKNSDGTYSKYDAASGERLTNEFFTTGDNHWYYIGANGKTVTGEVKIGDDTYFFAKDGKQLKGQIVTTRSGRISYYFGDSGKKAISTWVEIQPGVFVFFDKNGLAYPPENMN

SEQ ID NO: 14 - protein, 1341 aa, Streptococcus salivarius
MTDGKYYYVNEDGSHKENFAITVNGQLLYFGKDGALTSSSTHSFTPGTTNIVDGFSINNRAYDSSEASFELINGYLTADSWYRPVSIIKDGVTWQASTAEDFRPLLMAWWPNVDTQVNYLNYMSKVFNLEAKYTSTDKQADLNRAAKDIQVKIEQKIQAEKSTQWLRETISAFVKTQPQWNKETENYSKGGGEDHLQGGALLYVNDSRTPWANSNYRLLNRTATNQTGTINKSVLDEQSDPNHMGGFDFLLANDVDLSNPVVQAEQLNQIHYLMNWGSIVMGDKDANFDGIRVDAVDNVNADMLQLYTNYFREYYGVNKSEAQALAHISVLEAWSLNDNHYNDKTDGAALAMENKQRLALLFSLAKPIKDRTPAVSPLYNNTFNTTQRDFKTDWINKDGSTAYNEDGTAKQSTIGKYNEKYGDASGNYVFIRAHDNNVQDIIAEIIKKEINKKSDGFTISDSEMKQAFEIYNKDMLSSNKKYTLNNIPAAYAVMLQNMETITRVYYGDLYTDDGHYMETKSPYHDTIVNLMKNRIKYVSGGQAQRSYWLPTDGKMDNSDVELYRTSEVYTSVRYGKDIMTADDTEGSKYSRTSGQVTLVVNNPKLTLHESAKLNVEMGKIHANQKYRALIVGTADGIKNFTSDAEAIAAGYVKETDSNGVLTFGANDIKGYETFDMSGFVAVWVPVGASDDQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANSKSSGRDYQAQYGGEFLAELKAKYPKMFTENMISTGKPIDDSVKLKQWKAKYFNGTNVLDRGVGYVLSDEATGKYFTVTKEGNFIPLQLTGNEKAVTGFSNDGKGITYFGTSGNQAKSAFVTFNGNTYYFDARGHMVTNGEYSPNGKDVYRFLPNGIMLSNAFYVDANGNTYLYNYKGQMYKGGYTKFDVTETDKDGNESKVVKFRYFTNEGVMAKGLTVIDGSTQYFGEDGFQTKDKLATYKGKTYYFEAHTGNAIKNTWRNIDGKWYHFDENGVAATGAQVINGQKLYFNEDGSQVKGGVVKNADGTYSKYKEGSGELVTNEFFTTDGNVWYYAGADGKTVTGAQVINGQHLYFKEDGSQVKGGVVKNADGTYSKYDAATGERLTNEFFTTGDNNWYYIGSNGKTVTGEVKIGADTYYFAKDGKQVKGQTVTAGNGRISYYYGDSGKKAISTWIEIQPGIYVYFDKTGIAYPPRVLN

SEQ ID NO: 15 - protein, 1341 aa, Streptococcus salivarius
MIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYTSTDKQETLNVAAKDIQVKIEQKIQAEKSTQWLRETISAFVKTQPQWNKETENYSKGGGEDHLQGGALLYVNDSRTPWANSNYRLLNHTATNQKGTIDKSVLDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKKAFEIYNKDMLSSDKKYTLNNIPAAYAVMLQNMETITRVYYGDLYTDNGNYMETKSPYYDTIVNLMKNRIKYVSGGQAQRSYWLPTDGKMDNSDVELYRTNEVYASVRYGKDIMTADDTEGSKYSRTSGQVTLVANNPKLTLDQSAKLKVEMGKIHANQKYRALIVGTADGIKNFTSDADAIAAGYVKETDSNGVLTFGANDIKGYETFDMSGFVAVWVPVGASDDQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANTKSSGKDYQAKYGGEFLAELKAKYPEMFKVNMISTGKPIDDSVKLKQWKAEYFNGTNVLERGVGYVLSDEATGKYFTVTKDGNFIPLQLTGNEKVVTGFSNDGKGITYFGTSGTQAKSAFVTFNGNTYYFDARGHMVTNGEYSPNGKDVYRFLPNGIMLSNAFYVDANGNTYLYNSKGQMYKGGYTKFDVTETDKDGKESKVVKFRYFTNEGVMAKGVTVIDGFTQYFGEDGFQAKDKLVTFKGKTYYFDAHTGNAIKNTWRNIDGKWYHFDANGVAATGAQVINGQKLYFNEDGSQVKGGVVKNADGTYSKYKEGSGELVTNEFFTTDGNVWYYAGANGKTVTGAQVINGQHLYFNADGSQVKGGVVKNADGTYSKYDAATGERLTNEFFTTGDNNWYYIGANGKTVTGEVKIGDDTYYFAKDGKQVKGQTVSAGNGRISYYYGDSGKRAVSTWVEIQPGVYVYFDKNGLAYPPRVLN

(19) United States
(12) Patent Application Publication
(10) Pub. No.: US 2021/0282422 A1
(43) Pub. Date: Sep. 16, 2021

(54) USE OF GLUCOSYL TRANSFERASE TO PROVIDE IMPROVED TEXTURE IN FERMENTED MILK BASED PRODUCTS

(71) Applicant: DUPONT NUTRITION BIOSCIENCES APS, Copenhagen K (DK)

(72) Inventors: Charlotte Horsmans POULSEN, Brabrand (DK); Hans Christian BEJDER, Brabrand (DK); Morten Krog LARSEN, Sabro (DK); Signe Munk RYDTOFT, AARHUS C (DK)

(21) Appl. No.: 17/258,067

(22) PCT Filed: Jul. 3, 2019
(86) PCT No.: PCT/US19/40458

§ 371 Date: Jan. 5, 2021

Related U.S. Application Data
(60) Provisional application No. 62/694,061, filed on Jul. 5, 2018.

(30) Foreign Application Priority Data
Jun. 27, 2019 (US) PCT/US2019/039447

(51) Int. Cl. A23C 9/12; A23C 9/13; A23C 9/127

(52) U.S. Cl. CPC A23C 9/1216; C12Y 204/01; A23C 9/1275; A23C 9/1307

(57) ABSTRACT

The present teachings provide a method of making a yogurt product having increased thickness having the steps of providing milk; adding sucrose to the milk to form sweetened milk; contacting the sweetened milk with a glucosyl transferase to form an insoluble glucose polymer; inoculating with a starter culture; and fermenting to provide the yogurt product having increased thickness. Additional methods are provided.

### BACKGROUND OF THE INVENTION

Texture is a key quality and value parameter of fresh fermented dairy products, such as yogurts and fermented milks. Yogurt texture as relates to consumer eating sensation heavily impacts consumer perception. Today, stabilizers such as starch are common additives to yogurt to enhance texture. Yogurts containing starch, however, require special handling during processing so as not to lose the texture created by the starch through shear forces. The use of starch also adds to the expense of the yogurt. In addition, it is well known that starch negatively impacts yogurt in several ways. First, starch diminishes the “shininess” of yogurt, negatively impacting consumer visual perception. Moreover, added starch often leads to an undesirable sensory dryness of the yogurt.

In addition to starch, protein and fat levels in yogurt also contribute in a significant way to texture. Moreover, fat levels also impact taste. Modifying the protein level or fat level is a way to work with the cost profile and the nutritional profile of the yogurt. When reducing the content of any of these it is common to use other ingredients to compensate for texture or taste loss, typically by adding ingredients such as starch.

There is a need for adding texture to fermented dairy products that does not include the addition of starch or other stabilizers.

### SUMMARY OF THE INVENTION

In accordance with an aspect of the present invention, a method is presented of making a yogurt product having improved texture, improved texture being increased thickness and/or mouthfeel, having the steps of: providing milk; adding sucrose to the milk to form sweetened milk; contacting the sweetened milk with a glucosyl transferase to form an insoluble glucose polymer; inoculating with a starter culture; and fermenting to provide the yogurt product having improved texture which is increased thickness and/or increased mouthfeel.

Optionally, the milk is cow's milk. Optionally, the milk is selected from the group consisting of raw milk, pre-pasteurized milk, whole milk, skim milk, reconstituted milk, lactase treated milk, reduced lactose milk, lactose free milk and condensed milk. Optionally, the milk is raw milk.

Optionally, the method has the additional steps of homogenizing and pasteurizing the milk. Optionally, the step of contacting with glucosyl transferase is performed after the steps of homogenizing and pasteurizing. Optionally, the step of contacting with glucosyl transferase is performed before the steps of homogenizing and pasteurizing.

Optionally, the sucrose is added to constitute about 0.1 to 12% (w/w). Optionally, the sucrose is added to constitute about 2 to 8% (w/w). Optionally, the sucrose is added to constitute about 4 to 6% (w/w).

Optionally, the glucosyl transferase is an enzyme which has at least 70% sequence identity to an enzyme selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO: 12), GTF2919 (SEQ ID NO: 13), GTF2678 (SEQ ID NO; 14), and GTF3929 (SEQ ID NO: 15). Optionally, the glucosyl transferase is an enzyme which has at least 80% sequence identity to an enzyme selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO. 12), GTF2919 (SEQ ID NO: 13), GTF2678 (SEQ ID NO; 14), and GTF3929 (SEQ ID NO: 15). Optionally, the glucosyl transferase is an enzyme which has at least 90% sequence identity to an enzyme selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO: 12), GTF2919 (SEQ ID NO: 13), GTF2678 (SEQ ID NO; 14), and GTF3929 (SEQ ID NO: 15). Optionally, the glucosyl transferase is an enzyme which has at least 95% sequence identity to an enzyme selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO: 12), GTF2919 (SEQ ID NO: 13), GTF2678 (SEQ ID NO; 14), and GTF3929 (SEQ ID NO: 15). Optionally, the glucosyl transferase is selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO. 12), GTF2919 (SEQ ID NO: 13), GTF2678 (SEQ ID NO; 14), and GTF3929 (SEQ ID NO: 15). Optionally, the glucosyl transferase is GTFJ (SEQ ID NO: 1).

Optionally, the glucosyl transferase is present in the milk in an amount from about 0.005 mg per 100 ml milk to 15 mg per 100 ml milk. Optionally, the glucosyltransferase is present in an amount from about 0.03 mg per 100 ml milk to about 12.5 mg per 100 ml milk.

Optionally, the GTFJ is present in an amount from about 0.033 mg per 100 ml milk to about 12.5 mg per 100 ml milk. Optionally, the GTFJ is present in an amount from about 0.3 mg per 100 ml milk to about 5.0 mg per 100 ml milk.

Optionally, the glucosyl transferase is GTF300 (SEQ ID NO: 2). Optionally, the GTF300 is present in an amount from about 0.033 mg per 100 ml to about 12.5 mg per 100 ml milk. Optionally, the GTF300 is present in an amount from about 1.25 mg per 100 ml milk to about 5 mg per 100 ml milk.

Optionally, the increased texture is increased thickness. Optionally, the thickness is increased by 30% or more as compared with a control sample (no GTF enzyme). Optionally, the thickness is increased by 50% or more. Optionally, the thickness is increased by 70% or more. Optionally, the thickness is increased by 90% or more. Optionally, the thickness is increased by 100% or more. Optionally, the thickness is increased by 110% or more. Optionally, the thickness is increased by 120% or more.

Optionally, the increased texture is increased mouthfeel. Optionally, the mouthfeel is increased by 30% or more as compared with a control sample (no GTF enzyme). Optionally, the mouthfeel is increased by 50% or more. Optionally, the mouthfeel is increased by 70% or more. Optionally, the mouthfeel is increased by 90% or more. Optionally, the mouthfeel is increased by 100% or more. Optionally, the mouthfeel is increased by 110% or more. Optionally, the mouthfeel is increased by 120% or more.

Optionally, the method includes the further steps of cooling the yogurt of to a temperature of between 5 and 10° C. to provide a chilled yogurt; and pouring the chilled yogurt into preformed containers. Optionally, the containers provide a single serving of yogurt.

Optionally, the milk is low fat milk to provide a low fat yogurt. Optionally, the milk is non-fat milk to provide a non-fat yogurt.

Optionally, the protein content of the milk is adjusted to at least about 3% (w/w). Optionally, the protein content of the milk is adjusted to at least about 3.5%. Optionally, the protein content of the milk is adjusted to at least about 3.7% (w/w). Optionally, the protein content of the milk is adjusted to at least about 3.8% (w/w). Optionally, the protein content of the milk is adjusted to at least about 3.9% (w/w). Optionally, the protein content of the milk is adjusted to at least about 4.0% (w/w).

In accordance with an aspect of the present invention, a yogurt is presented which is made according to any or the preceding methods. Optionally, the yogurt contains pectin.

### BRIEF DESCRIPTION OF THE DRAWINGS

FIG. 1 depicts a flow diagram for inoculation of milk with culture and treatment with GTF enzyme.

FIG. 2 depicts the thickness and mouthfeel of GTF300 treated and control yogurt after 5 days using three different starter cultures: FIG. 2A (YO-MIX 860), FIG. 2B (YO-MIX 495) and FIG. 2C (YO-MIX 465).

FIG. 3 depicts the thickness and mouthfeel of enzyme treated and control yogurt after 28 days with GTF300 addition at the same time as culture inoculation using three different starter cultures: FIG. 3A (YO-MIX 860), FIG. 3B (YO-MIX 495) and FIG. 3C (YO-MIX 465).

FIG. 4 depicts a flow diagram for addition of enzyme prior to pasteurization and homogenization.

FIG. 5 depicts the thickness and mouthfeel of GTF300 treated and control yogurt after 7 days with enzyme addition before pasteurization and homogenization using four different starter cultures: FIG. 5A (YO-MIX 495), FIG. 5B (YO-MIX 465), FIG. 5C (YO-MIX 860) and FIG. 5D (YO-MIX 204).

FIG. 6 depicts the thickness and mouthfeel of enzyme treated and control yogurt after 28 days with GTF300 addition before pasteurization and homogenization using four different starter cultures: FIG. 6A (YO-MIX 860), FIG. 6B (YO-MIX 495), FIG. 6C (YO-MIX 465) and FIG. 6D (YO-MIX 204).

FIG. 7A (2% sucrose) and 7B (4% sucrose) thickness and mouthfeel as evaluated with GTF300 addition before pasteurization and homogenization.

FIG. 8 depicts the effect of yogurt cooling to different temperatures on texture and mouthfeel generated by GTF300 after filling.

FIG. 9 depicts the effect of GTFJ on thickness and mouthfeel. FIG. 9A shows effect of GTFJ as function of enzyme concentration and FIG. 9B shows the effect of 3.7% protein plus GTFJ as compared with a 4% protein yogurt without enzyme.

FIG. 10 depicts an extract of the chromatogram of 5% sucrose in water or 5% sucrose and 5% lactose in water with GTF300 at a dose of 0.2% in water.

### REFERENCE TO SEQUENCE LISTING SUBMITTED ELECTRONICALLY

The official copy of the sequence listing is submitted electronically via EFS-Web as an ASCII formatted sequence listing with a file named 20180703_NB41287_ST25.txt created on Jul. 3, 2018 and having a size of 174 kilobytes and is filed concurrently with the specification. The sequence listing contained in this ASCII formatted document is part of the specification and is herein incorporated by reference in its entirety.

### BRIEF DESCRIPTION OF THE SEQUENCE IDS

SEQ ID NO: 1 is the amino acid sequence of GTFJ.

SEQ ID NO. 2 is the amino acid sequence of GTF300.

SEQ ID NO: 3 is the amino acid sequence of GTF0874.

SEQ ID NO: 4 is the amino acid sequence of GTF6855.

SEQ ID NO: 5 is the amino acid sequence of GTF2379.

SEQ ID NO: 6 is the amino acid sequence of GTF7527.

SEQ ID NO: 7 is the amino acid sequence of GTF1724.

SEQ ID NO: 8 is the amino acid sequence of GTF0544.

SEQ ID NO: 9 is the amino acid sequence of GTF5926.

SEQ ID NO: 10 is the amino acid sequence of GTF4297.

SEQ ID NO. 11 is the amino acid sequence of GTF5618.

SEQ ID NO: 12 is the amino acid sequence of GTF2765.

SEQ ID NO: 13 is the amino acid sequence of GTF2919.

SEQ ID NO: 14 is the amino acid sequence of GTF2678.

SEQ ID NO. 15 is the amino acid sequence of GTF3929.

### DETAILED DISCLOSURE OF THE INVENTION

### Detailed Description of the Inventions

The practice of the present teachings will employ, unless otherwise indicated, conventional techniques of molecular biology (including recombinant techniques), microbiology, cell biology, and biochemistry, which are within the skill of the art. Such techniques are explained fully in the literature, for example, Molecular Cloning: A Laboratory Manual, second edition (Sambrook et al., 1989); Oligonucleotide Synthesis (M. J. Gait, ed., 1984; Current Protocols in Molecular Biology (F. M. Ausubel et al., eds., 1994); PCR: The Polymerase Chain Reaction (Mullis et al., eds., 1994); Gene Transfer and Expression: A Laboratory Manual (Kriegler, 1990), and The Alcohol Textbook (Ingledew et al., eds., Fifth Edition, 2009), and Essentials of Carbohydrate Chemistry and Biochemistry (Lindhorste, 2007).

Unless defined otherwise herein, all technical and scientific terms used herein have the same meaning as commonly understood by one of ordinary skill in the art to which the present teachings belong. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this invention. Any methods and materials similar or equivalent to those described herein can be used in the practice or testing of the present teachings.

Numeric ranges provided herein are inclusive of the numbers defining the range.

### Definitions

As used herein, “alpha (1-3) glucan” refers to an oligo or polysaccharide containing alpha 1-3 bonds between glucose monomers.

The terms “glucosyl transferase”, “glucosyl transferase enzyme”, “GTF enzyme”, and “GTF” are used interchangeably herein. Glucosyl transferases catalyze the synthesis of high molecular weight D-glucose polymers named glucan from sucrose. GTF enzymes are classified under the glycoside hydrolase family 70 (GH70) according to the CAZy (Carbohydrate-Active EnZymes) database (Cantarel et al., Nucleic Acids Res. 37:D233-238, 2009).

The terms, “wild-type,” “parental,” or “reference,” with respect to a polypeptide, refer to a naturally-occurring polypeptide that does not include a man-made substitution, insertion, or deletion at one or more amino acid positions. Similarly, the terms “wild-type,” “parental,” or “reference,” with respect to a polynucleotide, refer to a naturally-occurring polynucleotide that does not include a man-made nucleotide change. However, note that a polynucleotide encoding a wild-type, parental, or reference polypeptide is not limited to a naturally-occurring polynucleotide, and encompasses any polynucleotide encoding the wild-type, parental, or reference polypeptide.

Reference to the wild-type polypeptide is understood to include the mature form of the polypeptide. A “mature” polypeptide or variant, thereof, is one in which a signal sequence is absent, for example, cleaved from an immature form of the polypeptide during or following expression of the polypeptide.

The term “variant,” with respect to a polypeptide, refers to a polypeptide that differs from a specified wild-type, parental, or reference polypeptide in that it includes one or more naturally-occurring or man-made substitutions, insertions, or deletions of an amino acid. Similarly, the term “variant,” with respect to a polynucleotide, refers to a polynucleotide that differs in nucleotide sequence from a specified wild-type, parental, or reference polynucleotide. The identity of the wild-type, parental, or reference polypeptide or polynucleotide will be apparent from context.

The term “recombinant,” when used in reference to a subject cell, nucleic acid, protein or vector, indicates that the subject has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding a glucosyl transferase is a recombinant vector.

The terms “recovered,” “isolated,” and “separated,” refer to a compound, protein (polypeptides), cell, nucleic acid, amino acid, or other specified material or component that is removed from at least one other material or component with which it is naturally associated as found in nature. An “isolated” polypeptides, thereof, includes, but is not limited to, a culture broth containing secreted polypeptide expressed in a heterologous host cell.

The term “polymer” refers to a series of monomer groups linked together. A polymer is composed of multiple units of a single monomer. As used herein the term “glucose polymer” refers to glucose units linked together as a polymer. As long as there are at least three glucose units, the glucose polymer may contain non-glucose sugars such as lactose or galactose.

The term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

The term “nucleic acid” encompasses DNA, RNA, heteroduplexes, and synthetic molecules capable of encoding a polypeptide. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

The terms “transformed,” “stably transformed,” and “transgenic,” used with reference to a cell means that the cell contains a non-native (e.g., heterologous) nucleic acid sequence integrated into its genome or carried as an episome that is maintained through multiple generations.

The term “introduced” in the context of inserting a nucleic acid sequence into a cell, means “transfection”, “transformation” or “transduction,” as known in the art.

A “host strain” or “host cell” is an organism into which an expression vector, phage, virus, or other DNA construct, including a polynucleotide encoding a polypeptide of interest (e.g., a glucosyl transferase) has been introduced. Exemplary host strains are microorganism cells (e.g., bacteria, filamentous fungi, and yeast) capable of expressing the polypeptide of interest. The term “host cell” includes protoplasts created from cells.

The term “heterologous” with reference to a polynucleotide or protein refers to a polynucleotide or protein that does not naturally occur in a host cell.

The term “endogenous” with reference to a polynucleotide or protein refers to a polynucleotide or protein that occurs naturally in the host cell.

The term “expression” refers to the process by which a polypeptide is produced based on a nucleic acid sequence. The process includes both transcription and translation.

A “selective marker” or “selectable marker” refers to a gene capable of being expressed in a host to facilitate selection of host cells carrying the gene. Examples of selectable markers include but are not limited to antimicrobials (e.g., hygromycin, bleomycin, or chloramphenicol) and/or genes that confer a metabolic advantage, such as a nutritional advantage on the host cell.

A “vector” refers to a polynucleotide sequence designed to introduce nucleic acids into one or more cell types. Vectors include cloning vectors, expression vectors, shuttle vectors, plasmids, phage particles, cassettes and the like.

An “expression vector” refers to a DNA construct comprising a DNA sequence encoding a polypeptide of interest, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequences.

A “signal sequence” is a sequence of amino acids attached to the N-terminal portion of a protein, which facilitates the secretion of the protein outside the cell. The mature form of an extracellular protein lacks the signal sequence, which is cleaved off during the secretion process.

“Biologically active” refers to a sequence having a specified biological activity, such an enzymatic activity.

The term “specific activity” refers to the number of moles of substrate that can be converted to product by an enzyme or enzyme preparation per unit time under specific conditions. Specific activity is generally expressed as units (U)/mg of protein.

As used herein, “percent sequence identity” means that a particular sequence has at least a certain percentage of amino acid residues identical to those in a specified reference sequence, when aligned using the CLUSTAL W algorithm with default parameters. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF.

Deletions are counted as non-identical residues, compared to a reference sequence. Deletions occurring at either termini are included. For example, a variant with five amino acid deletions of the C-terminus of the mature 617 residue polypeptide would have a percent sequence identity of 99% (612/617 identical residues×100, rounded to the nearest whole number) relative to the mature polypeptide. Such a variant would be encompassed by a variant having “at least 99% sequence identity” to a mature polypeptide.

“Fused” polypeptide sequences are connected, i.e., operably linked, via a peptide bond between two subject polypeptide sequences.

The term “filamentous fungi” refers to all filamentous forms of the subdivision Eumycotina, particularly Pezizomycotina species.

The term “about” refers to ±5% to the referenced value.

“Lactase treated milk” means milk treated with lactase to reduce the amount of lactose sugar.

“Reduced lactose milk” means milk wherein the percentage of lactose is about 2% or lower.

“Lactose free milk” means milk wherein the percentage of lactose is about 0.5% or lower.

The terms “GTFJ” means the glucosyl transferase enzyme having the sequence as set forth in SEQ ID NO: 1.

The term “GTF300” means the glucosyl transferase having the sequence as set forth in SEQ ID NO: 2.

The term “texture” as used herein to refer to a yogurt or fermented milk products means the thickness of the yogurt and/or the sensory perception of mouthfeel or both. An “improvement” in texture means an increase in thickness and/or an increase in the sensory perception of mouthfeel or both. Unless otherwise noted, as used herein the “thickness” of a yogurt or fermented milk beverage means the apparent viscosity extracted at shear rate of 10 Hz. Thus, an increase in apparent viscosity at a shear rate of 10 Hz indicates an increase in thickness. The apparent viscosity extracted at shear rate 200 Hz is correlated to “mouthfeel”. Hence, an increase in apparent viscosity at a shear rate of 200 Hz indicates an increase in mouthfeel.

Additional Mutations

In some embodiments, the present glucosyl transferases further include one or more mutations that provide a further performance or stability benefit. Exemplary performance benefits include but are not limited to increased thermal stability, increased storage stability, increased solubility, an altered pH profile, increased specific activity, modified substrate specificity, modified substrate binding, modified pH-dependent activity, modified pH-dependent stability, increased oxidative stability, and increased expression. In some cases, the performance benefit is realized at a relatively low temperature. In some cases, the performance benefit is realized at relatively high temperature.

Furthermore, the present glucosyl transferases may include any number of conservative amino acid substitutions. Exemplary conservative amino acid substitutions are listed in the following Table.

Conservative Amino Acid Substitutions

| For Amino Acid | Code | Replace with any of |
|---|---|---|
| Alanine | A | D-Ala, Gly, beta-Ala, L-Cys, D-Cys |
| Arginine | R | D-Arg, Lys, D-Lys, homo-Arg, D-homo-Arg, Met, Ile, |
| | | D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, b-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, Met, D- |
| | | Met, Ile, D-Ile, Orn, D-Orn |
| Methionine | M | D-Met, S-Me-Cys, Ile, D-Ile, Leu, D-Leu, Val, D-Val |
| Phenylalanine | F | D-Phe, Tyr, D-Thr, L-Dopa, His, D-His, Trp, D-Trp, |
| | | Trans-3,4, or 5-phenylproline, cis-3,4, |
| | | or 5-phenylproline |
| Proline | P | D-Pro, L-I-thioazolidine-4-carboxylic acid, D-or L-1- |
| | | oxazolidine-4-carboxylic acid |
| Serine | S | D-Ser, Thr, D-Thr, allo-Thr, Met, D-Met, Met(O), D- |
| | | Met(O), L-Cys, D-Cys |
| Threonine | T | D-Thr, Ser, D-Ser, allo-Thr, Met, |
| | | D-Met, Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

The reader will appreciate that some of the above mentioned conservative mutations can be produced by genetic manipulation, while others are produced by introducing synthetic amino acids into a polypeptide by genetic or other means.

The present glucosyl transferases may be “precursor,” “immature,” or “full-length,” in which case they include a signal sequence, or “mature,” in which case they lack a signal sequence. Mature forms of the polypeptides are generally the most useful. Unless otherwise noted, the amino acid residue numbering used herein refers to the mature forms of the respective glucosyl transferase polypeptides. The present glucosyl transferase polypeptides may also be truncated to remove the N or C-termini, so long as the resulting polypeptides retain glucosyl transferase activity.

The present glucosyl transferases may be a “chimeric” or “hybrid” polypeptide, in that it includes at least a portion of a first glucosyl transferase polypeptide, and at least a portion of a second glucosyl transferase polypeptide. The present glucosyl transferases may further include heterologous signal sequence, an epitope to allow tracking or purification, or the like. Exemplary heterologous signal sequences are from B. licheniformis amylase (LAT), B. subtilis (AmyE or AprE), and Streptomyces CelA.

Production of Glucosyl Transferases

The present glucosyl transferases can be produced in host cells, for example, by secretion or intracellular expression. A cultured cell material (e.g., a whole-cell broth) comprising a glucosyl transferase can be obtained following secretion of the glucosyl transferase into the cell medium. Optionally, the glucosyl transferase can be isolated from the host cells, or even isolated from the cell broth, depending on the desired purity of the final glucosyl transferase. A gene encoding a glucosyl transferase can be cloned and expressed according to methods well known in the art. Suitable host cells include bacterial, fungal (including yeast and filamentous fungi), and plant cells (including algae). Particularly useful host cells include Aspergillus niger, Aspergillus oryzae or Trichoderma reesei. Other host cells include bacterial cells, e.g., Bacillus subtilis or B. licheniformis, as well as Streptomyces, and E. Coli.

The host cell further may express a nucleic acid encoding a homologous or heterologous glucosyl transferase, i.e., a glucosyl transferase that is not the same species as the host cell, or one or more other enzymes. The glucosyl transferase may be a variant glucosyl transferase. Additionally, the host may express one or more accessory enzymes, proteins, peptides.

Vectors

A DNA construct comprising a nucleic acid encoding a glucosyl transferase can be constructed to be expressed in a host cell. Because of the well-known degeneracy in the genetic code, variant polynucleotides that encode an identical amino acid sequence can be designed and made with routine skill. It is also well-known in the art to optimize codon use for a particular host cell. Nucleic acids encoding glucosyl transferase can be incorporated into a vector. Vectors can be transferred to a host cell using well-known transformation techniques, such as those disclosed below.

The vector may be any vector that can be transformed into and replicated within a host cell. For example, a vector comprising a nucleic acid encoding a glucosyl transferase can be transformed and replicated in a bacterial host cell as a means of propagating and amplifying the vector. The vector also may be transformed into an expression host, so that the encoding nucleic acids can be expressed as a functional glucosyl transferase. Host cells that serve as expression hosts can include filamentous fungi, for example.

A nucleic acid encoding a glucosyl transferase can be operably linked to a suitable promoter, which allows transcription in the host cell. The promoter may be any DNA sequence that shows transcriptional activity in the host cell of choice and may be derived from genes encoding proteins either homologous or heterologous to the host cell. Exemplary promoters for directing the transcription of the DNA sequence encoding a glucosyl transferase, especially in a bacterial host, are the promoter of the lac operon of E. coli, the Streptomyces coelicolor agarase gene dagA or celA promoters, the promoters of the Bacillus licheniformis α-amylase gene (amyL), the promoters of the Bacillus stearothermophilus maltogenic amylase gene (amyM), the promoters of the Bacillus amyloliquefaciens α-amylase (amyQ), the promoters of the Bacillus subtilis xylA and xylB genes etc. For transcription in a fungal host, examples of useful promoters are those derived from the gene encoding Aspergillus oryzae TAKA amylase, Rhizomucor miehei aspartic proteinase, Aspergillus niger neutral α-amylase, A. niger acid stable α-amylase, A. niger glucoamylase, Rhizomucor miehei lipase, A. oryzae alkaline protease, A. oryzae triose phosphate isomerase, or A. nidulans acetamidase. When a gene encoding a glucosyl transferaseis expressed in a bacterial species such as E. coli, a suitable promoter can be selected, for example, from a bacteriophage promoter including a T7 promoter and a phage lambda promoter. Examples of suitable promoters for the expression in a yeast species include but are not limited to the Gal 1 and Gal 10 promoters of Saccharomyces cerevisiae and the Pichia pastoris AOX1 or AOX2 promoters. cbh1 is an endogenous, inducible promoter from T. reesei. See Liu et al. (2008) “Improved heterologous gene expression in Trichoderma reesei by cellobiohydrolase I gene (cbh1) promoter optimization,” Acta Biochim. Biophys. Sin (Shanghai) 40(2): 158-65.

The coding sequence can be operably linked to a signal sequence. The DNA encoding the signal sequence may be the DNA sequence naturally associated with the glucosyl transferase gene to be expressed or from a different Genus or species. A signal sequence and a promoter sequence comprising a DNA construct or vector can be introduced into a fungal host cell and can be derived from the same source. For example, the signal sequence is the cbh1 signal sequence that is operably linked to a cbh1 promoter.

An expression vector may also comprise a suitable transcription terminator and, in eukaryotes, polyadenylation sequences operably linked to the DNA sequence encoding a variant glucosyl transferase. Termination and polyadenylation sequences may suitably be derived from the same sources as the promoter.

The vector may further comprise a DNA sequence enabling the vector to replicate in the host cell. Examples of such sequences are the origins of replication of plasmids pUC19, pACYC177, pUB110, pE194, pAMB1, and pIJ702.

The vector may also comprise a selectable marker, e.g., a gene the product of which complements a defect in the isolated host cell, such as the dal genes from B. subtilis or B. licheniformis, or a gene that confers antibiotic resistance such as, e.g., ampicillin, kanamycin, chloramphenicol, or tetracycline resistance. Furthermore, the vector may comprise Aspergillus selection markers such as amdS, argB, niaD and xxsC, a marker giving rise to hygromycin resistance, or the selection may be accomplished by co-transformation, such as known in the art. See e.g., International PCT Application WO 91/17243.

Intracellular expression may be advantageous in some respects, e.g., when using certain bacteria or fungi as host cells to produce large amounts of glucosyl transferase for subsequent enrichment or purification. Extracellular secretion of glucosyl transferase into the culture medium can also be used to make a cultured cell material comprising the isolated glucosyl transferase.

The expression vector typically includes the components of a cloning vector, such as, for example, an element that permits autonomous replication of the vector in the selected host organism and one or more phenotypically detectable markers for selection purposes. The expression vector normally comprises control nucleotide sequences such as a promoter, operator, ribosome binding site, translation initiation signal and optionally, a repressor gene or one or more activator genes. Additionally, the expression vector may comprise a sequence coding for an amino acid sequence capable of targeting the glucosyl transferaseto a host cell organelle such as a peroxisome, or to a particular host cell compartment. Such a targeting sequence includes but is not limited to the sequence, SKL. For expression under the direction of control sequences, the nucleic acid sequence of the glucosyl transferase is operably linked to the control sequences in proper manner with respect to expression.

The procedures used to ligate the DNA construct encoding a glucosyl transferase, the promoter, terminator and other elements, respectively, and to insert them into suitable vectors containing the information necessary for replication, are well known to persons skilled in the art (see, e.g., Sambrook et al., MOLECULAR CLONING: A LABORATORY MANUAL, 2^{nd} ed., Cold Spring Harbor, 1989, and 3^{rd} ed., 2001).

Transformation and Culture of Host Cells

An isolated cell, either comprising a DNA construct or an expression vector, is advantageously used as a host cell in the recombinant production of a glucosyl transferase. The cell may be transformed with the DNA construct encoding the enzyme, conveniently by integrating the DNA construct (in one or more copies) in the host chromosome. This integration is generally considered to be an advantage, as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector as described above in connection with the different types of host cells.

Examples of suitable bacterial host organisms are Gram positive bacterial species such as Bacillaceae including Bacillus subtilis, Bacillus licheniformis, Bacillus lentus, Bacillus brevis, Geobacillus (formerly Bacillus) stearothermophilus, Bacillus alkalophilus, Bacillus amyloliquefaciens, Bacillus coagulans, Bacillus lautus, Bacillus megaterium, and Bacillus thuringiensis; Streptomyces species such as Streptomyces murinus; lactic acid bacterial species including Lactococcus sp. such as Lactococcus lactis; Lactobacillus sp. including Lactobacillus reuteri; Leuconostoc sp.; Pediococcus sp.; and Streptococcus sp. Alternatively, strains of a Gram negative bacterial species belonging to Enterobacteriaceae including E. coli, or to Pseudomonadaceae can be selected as the host organism.

A suitable yeast host organism can be selected from the biotechnologically relevant yeasts species such as but not limited to yeast species such as Pichia sp., Hansenula sp., or Kluyveromyces, Yarrowinia, Schizosaccharomyces species or a species of Saccharomyces, including Saccharomyces cerevisiae or a species belonging to Schizosaccharomyces such as, for example, S. pombe species. A strain of the methylotrophic yeast species, Pichia pastoris, can be used as the host organism. Alternatively, the host organism can be a Hansenula species. Suitable host organisms among filamentous fungi include species of Aspergillus, e.g., Aspergillus niger, Aspergillus oryzae, Aspergillus tubigensis, Aspergillus awamori, or Aspergillus nidulans. Alternatively, strains of a Fusarium species, e.g., Fusarium oxysporum or of a Rhizomucor species such as Rhizomucor miehei can be used as the host organism. Other suitable strains include Thermomyces and Mucor species. In addition, Trichoderma sp. can be used as a host. A suitable procedure for transformation of Aspergillus host cells includes, for example, that described in EP 238023. A glucosyl transferase expressed by a fungal host cell can be glycosylated, i.e., will comprise a glycosyl moiety. The glycosylation pattern can be the same or different as present in the wild-type glucosyl transferase. The type and/or degree of glycosylation may impart changes in enzymatic and/or biochemical properties.

It is advantageous to delete genes from expression hosts, where the gene deficiency can be cured by the transformed expression vector. Known methods may be used to obtain a fungal host cell having one or more inactivated genes. Gene inactivation may be accomplished by complete or partial deletion, by insertional inactivation or by any other means that renders a gene nonfunctional for its intended purpose, such that the gene is prevented from expression of a functional protein. A gene from a Trichoderma sp. or other filamentous fungal host that has been cloned can be deleted, for example, cbh1, cbh2, egl1, and egl2 genes. Gene deletion may be accomplished by inserting a form of the desired gene to be inactivated into a plasmid by methods known in the art.

Introduction of a DNA construct or vector into a host cell includes techniques such as transformation; electroporation; nuclear microinjection; transduction; transfection, e.g., lipofection mediated and DEAE-Dextrin mediated transfection; incubation with calcium phosphate DNA precipitate; high velocity bombardment with DNA-coated microprojectiles; and protoplast fusion. General transformation techniques are known in the art. See, e.g., Sambrook et al. (2001), supra. The expression of heterologous protein in Trichoderma is described, for example, in U.S. Pat. No. 6,022,725. Reference is also made to Cao et al. (2000) Science 9:991-1001 for transformation of Aspergillus strains. Genetically stable transformants can be constructed with vector systems whereby the nucleic acid encoding a glucosyl transferase is stably integrated into a host cell chromosome. Transformants are then selected and purified by known techniques.

Expression

A method of producing a glucosyl transferase may comprise cultivating a host cell as described above under conditions conducive to the production of the enzyme and recovering the enzyme from the cells and/or culture medium.

The medium used to cultivate the cells may be any conventional medium suitable for growing the host cell in question and obtaining expression of a glucosyl transferase. Suitable media and media components are available from commercial suppliers or may be prepared according to published recipes (e.g., as described in catalogues of the American Type Culture Collection).

An enzyme secreted from the host cells can be used in a whole broth preparation. In the present methods, the preparation of a spent whole fermentation broth of a recombinant microorganism can be achieved using any cultivation method known in the art resulting in the expression of a glucosyl transferase. Fermentation may, therefore, be understood as comprising shake flask cultivation, small- or large-scale fermentation (including continuous, batch, fed-batch, or solid state fermentations) in laboratory or industrial fermenters performed in a suitable medium and under conditions allowing the glucosyl transferase to be expressed or isolated. The term “spent whole fermentation broth” is defined herein as unfractionated contents of fermentation material that includes culture medium, extracellular proteins (e.g., enzymes), and cellular biomass. It is understood that the term “spent whole fermentation broth” also encompasses cellular biomass that has been lysed or permeabilized using methods well known in the art.

An enzyme secreted from the host cells may conveniently be recovered from the culture medium by well-known procedures, including separating the cells from the medium by centrifugation or filtration, and precipitating proteinaceous components of the medium by means of a salt such as ammonium sulfate, followed by the use of chromatographic procedures such as ion exchange chromatography, affinity chromatography, or the like.

The polynucleotide encoding a glucosyl transferase in a vector can be operably linked to a control sequence that is capable of providing for the expression of the coding sequence by the host cell, i.e. the vector is an expression vector. The control sequences may be modified, for example by the addition of further transcriptional regulatory elements to make the level of transcription directed by the control sequences more responsive to transcriptional modulators. The control sequences may in particular comprise promoters.

Host cells may be cultured under suitable conditions that allow expression of a glucosyl transferase. Expression of the enzymes may be constitutive such that they are continually produced, or inducible, requiring a stimulus to initiate expression. In the case of inducible expression, protein production can be initiated when required by, for example, addition of an inducer substance to the culture medium, for example dexamethasone or IPTG or Sophorose. Polypeptides can also be produced recombinantly in an in vitro cell-free system, such as the TNT™ (Promega) rabbit reticulocyte system.

Methods for Enriching and Purifying Glucosyl Transferases

Fermentation, separation, and concentration techniques are well known in the art and conventional methods can be used in order to prepare a glucosyl transferase polypeptide-containing solution.

After fermentation, a fermentation broth is obtained, the microbial cells and various suspended solids, including residual raw fermentation materials, are removed by conventional separation techniques in order to obtain a glucosyl transferase solution. Filtration, centrifugation, microfiltration, rotary vacuum drum filtration, ultrafiltration, centrifugation followed by ultra-filtration, extraction, or chromatography, or the like, are generally used.

It is desirable to concentrate a glucosyl transferase polypeptide-containing solution in order to optimize recovery. Use of unconcentrated solutions requires increased incubation time in order to collect the enriched or purified enzyme precipitate.

The enzyme containing solution is concentrated using conventional concentration techniques until the desired enzyme level is obtained. Concentration of the enzyme containing solution may be achieved by any of the techniques discussed herein. Exemplary methods of enrichment and purification include but are not limited to rotary drum vacuum filtration and/or ultrafiltration.

GTF Enzymes

Glucan polymers produced by adding a GTF enzyme to an appropriate solution of sucrose can be soluble or insoluble. Solubility of glucan depends on a number of factors, including percent of alpha 1,3 linkages, percent of alpha 1,6 linkages and polymer length (DPn). See, e.g., U.S. Pat. No. 8,871,474, incorporated herein by reference in its entirety (the '474 patent).

Other products (byproducts) of GTF include glucose (where glucose is hydrolyzed from the glucosyl-GTF enzyme intermediate complex), various soluble oligosaccharides (DP2-DP7), and leucrose (where glucose of the glucosyl-gtf enzyme intermediate complex is linked to fructose). Leucrose is a disaccharide composed of glucose and fructose linked by an alpha-1,5 linkage. Wild type forms of glucosyl transferase enzymes generally contain (in the N-terminal to C-terminal direction) a signal peptide, a variable domain, a catalytic domain, and a glucan-binding domain.

The glucosyl transferases in certain embodiments of the invention may be derived from a Streptococcus species, Leuconostoc species or Lactobacillus species, for example. Examples of Streptococcus species from which the glucosyl transferase may be derived include S. salivarius, S. sobrinus, S. dentirousetti, S. downei, S. mutans, S. oralis, S. gallolyticus and S. sanguinis. Examples of Leuconostoc species from which the glucosyl transferase may be derived include L. mesenteroides, L. amelibiosum, L. argentinum, L. carnosum, L. citreum, L. cremoris, L. dextranicum and L. fructosum. Examples of Lactobacillus species from which the glucosyl transferase may be derived include L. acidophilus, L. delbrueckii, L. helveticus, L. salivarius, L. casei, L. curvatus, L. plantarum, L. sakei, L. brevis, L. buchneri, L. fermentum and L. reuteri.

In accordance with an aspect of the present invention, it has been determined that GTF enzymes producing insoluble glucan are particularly preferred. Insoluble glucan is glucan which is not soluble in aqueous solutions. As set forth in the '474 patent, insoluble glucan polymers tend to have a relatively high percentage of alpha 1,3 linkages to alpha 1,6 linkages and a DPn of at least 100. In accordance with an aspect of the present invention, the following GTF enzymes can be used to form insoluble glucan polymers: GTFJ, GTF300, GTF0874, 6855, 2379, 7527, 1724, 0544, 5926, 4297, 5618, 2765, 0427, 2919, 2678, and 3929.

| SEQ | | |
|---|---|---|
| ID | | |
| NO: | Sequence | Origin |
| 1 | MDETQDKTVTQSNSGTTASLVTSPEATKEADKRTNTKEADVLTPAKETNAVETATTTNTQ | Unknown |
| | ATAEAATTATTADVAVAAVPNKEAVVTTDAPAVTTEKAEEQPATVKAEVVNTEVKAPEAA | streptococcus |
| | LKDSEVEAALSLKNIKNIDGKYYYVNEDGSHKENFAITVNGQLLYFGKDGALTSSSTYSF | species |
| | TPGTTNIVDGFSINNRAYDSSEASFELIDGYLTADSWYRPASIIKDGVTWQASTAEDFRP | |
| | LLMAWWPNVDTQVNYLNYMSKVFNLDAKYSSTDKQETLKVAAKDIQIKIEQKIQAEKSTQ | |
| | WLRETISAFVKTQPQWNKETENYSKGGGEDHLQGGALLYVNDSRTPWANSDYRRLNRTAT | |
| | NQTGTIDKSILDEQSDPNHMGGFDFLLANDVDLSNPVVQAEQLNQIHYLMNWGSIVMGDK | |
| | DANFDGIRVDAVDNVDADMLQLYTNYFREYYGVNKSEANALAHISVLEAWSLNDNHYNDK | |
| | TDGAALAMENKQRLALLFSLAKPIKERTPAVSPLYNNTFNTTQRDEKTDWINKDGSKAYN | |
| | EDGTVKQSTIGKYNEKYGDASGNYVFIRAHDNNVQDIIAEIIKKEINPKSDGFTITDAEM | |
| | KQAFEIYNKDMLSSDKKYTLNNIPAAYAVMLQNMETITRVYYGDLYTDDGHYMETKSPYY | |
| | DTIVNLMKSRIKYVSGGQAQRSYWLPTDGKMDNSDVELYRTNEVYTSVRYGKDIMTANDT | |
| | EGSKYSRTSGQVTLVANNPKLNLDQSAKLNVEMGKIHANQKYRALIVGTADGIKNFTSDA | |
| | DAIAAGYVKETDSNGVLTFGANDIKGYETFDMSGFVAVWVPVGASDNQDIRVAPSTEAKK | |
| | EGELTLKATEAYDSQLIYEGFSNFQTIPDGSDPSVYTNRKIAENVDLFKSWGVTSFEMAP | |
| | QFVSADDGTFLDSVIQNGYAFADRYDLAMSKNNKYGSKEDLRDALEALHKAGIQAIADWV | |
| | PDQIYQLPGKEVVTATRTDGAGRKIADAIIDHSLYVANSKSSGKDYQAKYGGEFLAELKA | |
| | KYPEMFKVNMISTGKPIDDSVKLKQWKAEYFNGTNVLERGVGYVLSDEATGKYFTVTKEG | |
| | NFIPLQLTGKEKVITGFSSDGKGITYFGTSGTQAKSAFVTFNGNTYYFDARGHMVTNSEY | |
| | SPNGKDVYRFLPNGIMLSNAFYIDANGNTYLYNSKGQMYKGGYTKFDVSETDKDGKESKV | |
| | VKFRYFTNEGVMAKGVTVIDGFTQYFGEDGFQAEDKLVTFKGKTYYFDAHTGNGIKDTWR | |
| | NINGKWYYFDANGVAATGAQVINGQKLYFNEDGSQVKGGVVKNADGTYSKYKEGFGELVT | |
| | NEFFTTDGNVWYYAGANGKTVTGAQVINGQHLYFNADGSQVKGGVVKNADGTYSKYNAST | |
| | GERLTNEFFTTGDNNWYYIGANGKSVTGEVKIGDDTYFFAKDGKQVKGQTVSAGNGRISY | |
| | YYGDSGKRAVSTWIEIQPGVYVYFDKNGLAYPPRVLN | |
| 2 | MIDGKYYYVNEDGSHKENFAITVNGQLLYFGKDGALTSSSTYSFTPGTTNIVDGFSINNR | Unknown |
| | AYDSSEASFELIDGYLTADSWYRPASIIKDGVTWQASTAEDFRPLLMAWWPNVDTQVNYL | streptococcus |
| | NYMSKVFNLDAKYSSTDKQETLKVAAKDIQIKIEQKIQAEKSTQWLRETISAFVKTQPQW | species |
| | NKETENYSKGGGEDHLQGGALLYVNDSRTPWANSDYRRLNRTATNQTGTIDKSILDEQSD | |
| | PNHMGGFDFLLANDVDLSNPVVQAEQLNQIHYLMNWGSIVMGDKDANFDGIRVDAVDNVD | |
| | ADMLQLYTNYFREYYGVNKSEANALAHISVLEDWSLNDNHYNDKTDGAALAMENKQRLAL | |
| | LFSLAKPIKERTPAVSPLYNNTFNTTQRDEKTDWINKDGSKAYNEDGTVKQSTIGKYNEK | |
| | YGDASGNYVYIRAHDNNVQDIIAEIIKKEINPKSDGFTITDAEMKQAFEIYNKDMLSSDK | |
| | KYTLNNIPAAYAVMLQNMETITRVYYGDLYTDDGHYMETKSPYYDTIVNLMKSRIKYVSG | |
| | GQAQRSYWLPTDGKMDNSDVELYSTNEVYTSVRYGKDIMTANDTEGSKYSRTSGQVTLVA | |
| | NNPKLTLDQSAKLNVEMGKIHANQKYRALIVGTADGIKNFTSDADAIAAGYVKETDSNGV | |
| | LTFGANDIKGYETFDMSGFVAVWVPVGASDDQDIRVAPSTEAKKEGELTLKATEAYDSQL | |
| | IYEGFSNFQTIPDGSDPSVYTNRKIAENVDLFKSWGVTSFEMAPQFVSADGGTFLDSVIQ | |
| | NGYAFADRYDLAMSKNNKYGSKEDLRDALKALHKAGIQAIADWVPDQIYQLPGKEVVTAT | |
| | RTDGAGRKIADAIIDHSLYVANTKSSGKDYQAKYGGEFLAELKAKYPEMFKVNMISTGKP | |
| | IDDSVKLKQWKAEYFNGTNVLERGVGYVLSDEATGKYFTVTKDGNFIPLQLTGNEKVVTG | |
| | FSNDGKGITYFGTSGTQAKSAFVTFNGNTYYFDARGHMVTNGEYSPNGKDVYRFLPNGIM | |
| | LSNAFYVDANGNTYLYNSKGQMYKGGYTKFDVTETDKDGKESKVVKFRYFTNEGVMAKGV | |
| | TVIDGFTQYFGEDGFQAKDKLVTFKGKTYYFDAHTGNAIKDTWRNINGKWYHFDANGVAA | |
| | TGAQVINGQKLYFNEDGSQVKGGVVKNADGTYSKYKEGSGELVTNEFFTTDGNVWYYAGA | |
| | NGKTVTGAQVINGQHLYFNADGSQVKGGVVKNADGTYSKYDASTGERLTNEFFTTGDNNW | |
| | YYIGANGKSVTGEVKIGDDTYFFAKDGKQVKGQTVSAGNGRISYYYGDSGKRAVSTWIEI | |
| | QPGVYVYFDKNGIAYPPMILN | |
| 3 | MVDGKYYYYDQDGNVKKNFAVSVGDKIYYFDETGAYKDTSKVDADKSSSAVSQNATIFAA | Streptococcus |
| | NNRAYSTSAKNFEAVDNYLTADSWYRPKSILKDGKTWTESGKDDFRPLLMAWWPDTETKR | sobrinus |
| | NYVNYMNKVVGIDKTYTAETSQADLTAAAELVQARIEQKITSENNTKWLREAISAFVKTQ | |
| | PQWNGESEKPYDDHLQNGALLFDNQTDLTPDTQSNYRLLNRTPTNQTGSLDSRFTYNPND | |
| | PLGGYDFLLANDVDNSNPVVQAEQLNWLHYLLNFGSIYANDADANFDSIRVDAVDNVDAD | |
| | LLQISSDYLKAAYGIDKNNKNANNHVSIVEAWSDNDTPYLHDDGDNLMNMDNKFRLSMLW | |
| | SLAKPLDKRSGLNPLIHNSLVDREVDDREVETVPSYSFARAHDSEVQDIIRDIIKAEINP | |
| | NSFGYSFTQEEIEQAFKIYNEDLKKTDKKYTHYNVPLSYTLLLTNKGSIPRVYYGDMFTD | |
| | DGQYMANKTVNYDAIESLLKARMKYVSGGQAMQNYQIGNGEILTSVRYGKGALKQSDKGD | |
| | ATTRTSGVGVVMGNQPNFSLDGKVVALNMGAAHANQEYRALMVSTKDGVATYATDADASK | |
| | AGLVKRTDENGYLYFLNDDLKGVANPQVSGFLQVWVPVGAADDQDIRVAASDTASTDGKS | |
| | LHQDAAMDSRVMFEGFSNFQSFATKEEEYTNVVIANNVDKFVSWGITDFEMAPQYVSSTD | |
| | GQFLDSVIQNGYAFTDRYDLGMSKANKYGTADQLVKAIKALHAKGLKVMADWVPDQMYTF | |
| | PKQEVVTVTRTDKFGKPIAGSQINHSLYVTDTKSSGDDYQAKYGGAFLDELKEKYPELFT | |
| | KKQISTGQAIDPSVKIKQWSAKYFNGSNILGRGADYVLSDQVSNKYFNVASDTLFLPSSL | |
| | LGKVVESGIRYDGKGYIYNSSATGDQVKASFITEAGNLYYFGKDGYMVTGAQTINGANYF | |
| | FLENGTALRNTIYTDAQGNSHYYANDGKRYENGYQQFGNDWRYFKDGNMAVGLTTVDGNV | |
| | QYFDKDGVQAKDKIIVTRDGKVRYFDQHNGNAATNTFIADKTGHWYYLGKDGVAVTGAQT | |
| | VGKQKLYFEANGQQVKGDFVTSDEGKLYFYDVDSGDMWTDTFIEDKAGNWFYLGKDGAAV | |
| | TGAQTIRGQKLYFKANGQQVKGDIVKGTDGKIRYYDAKSGEQVFNKTVKAADGKTYVIGN | |
| | DGVAVDPSVVKGQTFKDASGALRFYNLKGQLVTGSGWYETANHDWVYIQSGKALTGEQTI | |
| | NGQHLYFKEDGHQVKGQLVTGTDGKVRYYDANSGDQAFNKSVTVNGKTYYFGNDGTAQTA | |
| | GNPKGQTFKDGSDIRFYSMEGQLVTGSGWYENAQGQWLYVKNGKVLTGLQTVGSQRVYFD | |
| | ENGIQAKGKAVRTSDGKIRYFDENSGSMITNQWKFVYGQYYYFGNDGARIYRGWN | |
| 4 | MIDGKYYYVNEDGSHKENFAITVNGQLLYFGKDGALTSSSTYSFTPGTTNIVDGFSINNR | Streptococcus |
| | AYDSSEASFELIDGYLTADSWYRPASIIKDGVTWQASTAEDFRPLLMAWWPNVDTQVNYL | salivarius |
| | NYMSKVFNLDAKYSSTDKQETLKVAAKDIQIKIEQKIQAEKSTQWLRETISAFVKTQPQW | |
| | NKETENYSKGGGEDHLQGGALLYVNDSRTPWANSDYRRLNRTATNQTGTIDKSILDEQSD | |
| | PNHMGGFDFLLANDVDLSNPVVQAEQLNQIHYLMNWGSIVMGDKDANFDGIRVDAVDNVD | |
| | ADMLQLYTNYFREYYGVNKSEANALAHISVLEAWSLNDNHYNDKTDGAALAMENKQRLAL | |
| | LFSLAKPIKERTPAVSPLYNNTFNTTQRDEKTDWINKDGSKAYNEDGTVKQSTIGKYNEK | |
| | YGDASGNYVFIRAHDNNVQDIIAEIIKKEINPKSDGFTITDAEMKQAFEIYNKDMLSSDK | |
| | KYTLNNIPAAYAVMLQNMETITRVYYGDLYTDDGHYMETKSPYYDTIVNLMKSRIKYVSG | |
| | GQAQRSYWLPTDGKMDNSDVELYRTNEVYTSVRYGKDIMTANDTEGSKYSRTSGQVTLVA | |
| | NNPKLTLDQSAKLNVEMGKIHANQKYRALIVGTADGIKNFTSDADAIAAGYVKETDSNGV | |
| | LTFGANDIKGYETFDMSGFVAVWVPVGASDDQDIRVAPSTEAKKEGELTLKATEAYDSQL | |
| | IYEGFSNFQTIPDGSDPSVYTNRKIAENVDLFKSWGVTSFEMAPQFVSADDGTFLDSVIQ | |
| | NGYAFADRYDLAMSKNNKYGSKEDLRDALKALHKAGIQAIADWVPDQIYQLPGKEVVTAT | |
| | RTDGAGRKIADAIIDHSLYVANTKSSGKDYQAKYGGEFLAELKAKYPEMFKVNMISTGKP | |
| | IDDSVKLKQWKAEYFNGTNVLERGVGYVLSDEATGKYFTVTKDGNFIPLQLTGNEKVVTG | |
| | FSNDGKGITYFGTSGTQAKSAFVTFNGNTYYFDARGHMVTNGEYSPNGKDVYRFLPNGIM | |
| | LSNAFYVDANGNTYLYNSKGQMYKGGYTKFDVTETDKDGKESKVVKFRYFTNEGVMAKGV | |
| | TVIDGFTQYFGEDGFQAKDKLVTFKGKTYYFDAHTGNAIKDTWRNINGKWYHFDANGVAA | |
| | TGAQVINGQKLYFNEDGSQVKGGVVKNADGTYSKYKEGSGELVTNEFFTTDGNVWYYAGA | |
| | NGKTVTGAQVINGQHLYFNADGSQVKGGVVKNADGTYSKYDASTGERLTNEFFTTGDNNW | |
| | YYIGANGKSVTGEVKIGDDTYFFAKDGKQVKGQTVSAGNGRISYYYGDSGKRAVSTWIEI | |
| | QPGVYVYFDKNGIAYPPRVLN | |
| 5 | MPSHIKTINGKQYYVEDDGTIRKNYVLERIGGSQYFNAETGELSNQKEYRFDKNGGTGSS | Streptococcus |
| | ADSTNTNVTVNGDKNAFYGTTDKDIELVDGYFTANTWYRPKEILKDGKEWTASTENDKRP | salivarius |
| | LLTVWWPSKAIQASYLNYMKEQGLGTNQTYTSFSSQTQMDQAALEVQKRIEERIAREGNT | |
| | DWLRTTIKNFVKTQPGWNSTSENLDNNDHLQGGALLYNNDSRTSHANSDYRLLNRTPTSQ | |
| | TGKHNPKYTKDTSNGGFEFLLANDIDNSNPAVQAEQLNWLHYIMNIGTITGGSEDENFDG | |
| | VRVDAVDNVNADLLQIASDYFKAKYGADQSQDQAIKHLSILEAWSHNDAYYNEDTKGAQL | |
| | PMDDPMHLALVYSLLRPIGNRSGVEPLISNSLNDRSESGKNSKRMANYAFVRAHDSEVQS | |
| | IIGQIIKNEINPQSTGNTFTLDEMKKAFEIYNKDMRSANKQYTQYNIPSAYALMLTHKDT | |
| | VPRVYYGDMYTDDGQYMAQKSPYYDAIETLLKGRIRYAAGGQDMKVNYIGYGNTNGWDAA | |
| | GVLTSVRYGTGANSASDTGTAETRNQGMAVIVSNQPALRLTSNLTINMGAAHRNQAYRPL | |
| | LLTTNDGVATYLNDSDANGIVKYTDGNGNLTFSANEIRGIRNPQVDGYLAVWVPVGASEN | |
| | QDVRVAPSKEKNSSGLVYESNAALDSQVIYEGFSNFQDFVQNPSQYTNKKIAENANLFKS | |
| | WGITSFEFAPQYVSSDDGSFLDSVIQNGYAFTDRYDIGMSKDNKYGSLADLKAALKSLHA | |
| | VGISAIADWVPDQIYNLPGDEVVTATRVNNYGETKDGAIIDHSLYAAKTRTFGNDYQGKY | |
| | GGAFLDELKRLYPQIFDRVQISTGKRMTTDEKITQWSAKYMNGTNILDRGSEYVLKNGLN | |
| | GYYGTNGGKVSLPKVVGSNQSTNGDNQNGDGSGKFEKRLFSVRYRYNNGQYAKNAFIKDN | |
| | DGNVYYFDNSGRMAVGEKTIDGKQYFFLANGVQLRDGYRQNRRGQVFYYDQNGVLNANGK | |
| | QDPKPDNNNNASGRNQFVQIGNNVWAYYDGNGKRVTGHQNINGQELFFDNNGVQVKGRTV | |
| | NENGAIRYYDANSGEMARNRFAEIEPGVWAYFNNDGTAVKGSQNINGQDLYFDQNGRQVK | |
| | GALANVDGNLRYYDVNSGELYRNRFHEIDGSWYYFDGNGNAVKGMVNINGQNLLFDNNGK | |
| | QIKGHLVRVNGVVRYFDPNSGEMAVNRWVEVSPGWWVYFDGEGRGQI | |
| 6 | MDETQDKTVTQSNSGTTASLVTSPEATKEADKRTNTKEADVLTPAKETNAVETATTTNTQ | Streptococcus |
| | ATAEAATTATTADVAVAAVPNKEAVVTTDAPAVTTEKAEEQPATVKAEVVNTEVKAPEAA | salivarius |
| | LKDSEVEAALSLKNIKNIDGKYYYVNEDGSHKENFAITVNGQLLYFGKDGALTSSSTYSF | |
| | TPGTTNIVDGFSINNRAYDSSEASFELIDGYLTADSWYRPASIIKDGVTWQASTAEDFRP | |
| | LLMAWWPNVDTQVNYLNYMSKVFNLDAKYSSTDKQETLKVAAKDIQIKIEQKIQAEKSTQ | |
| | WLRETISAFVKTQPQWNKETENYSKGGGEDHLQGGALLYVNDSRTPWANSDYRRLNRTAT | |
| | NQTGTIDKSILDEQSDPNHMGGFDFLLANDVDLSNPVVQAEQLNQIHYLMNWGSIVMGDK | |
| | DANFDGIRVDAVDNVDADMLQLYTNYFREYYGVNKSEANALAHISVLEAWSLNDNHYNDK | |
| | TDGAALAMENKQRLALLFSLAKPIKERTPAVSPLYNNTFNTTQRDEKTDWINKDGSKAYN | |
| | EDGTVKQSTIGKYNEKYGDASGNYVFIRAHDNNVQDIIAEIIKKEINPKSDGFTITDAEM | |
| | KQAFEIYNKDMLSSDKKYTLNNIPAAYAVMLQNMETITRVYYGDLYTDDGHYMETKSPYY | |
| | DTIVNLMKSRIKYVSGGQAQRSYWLPTDGKMDNSDVELYRTNEVYTSVRYGKDIMTANDT | |
| | EGSKYSRTSGQVTLVANNPKLNLDQSAKLNVEMGKIHANQKYRALIVGTADGIKNFTSDA | |
| | DAIAAGYVKETDSNGVLTFGANDIKGYETFDMSGFVAVWVPVGASDNQDIRVAPSTEAKK | |
| | EGELTLKATEAYDSQLIYEGFSNFQTIPDGSDPSVYTNRKIAENVDLFKSWGVTSFEMAP | |
| | QFVSADDGTFLDSVIQNGYAFADRYDLAMSKNNKYGSKEDLRDALKALHKAGIQAIADWV | |
| | PDQIYQLPGKEVVTATRTDGAGRKIADAIIDHSLYVANSKSSGKDYQAKYGGEFLAELKA | |
| | KYPEMFKVNMISTGKPIDDSVKLKQWKAEYFNGTNVLERGVGYVLSDEATGKYFTVTKEG | |
| | NFIPLQLTGKEKVITGFSSDGKGITYFGTSGTQAKSAFVTFNGNTYYFDARGHMVTNSEY | |
| | SPNGKDVYRFLPNGIMLSNAFYIDANGNTYLYNSKGQMYKGGYTKFDVSETDKDGKESKV | |
| | VKFRYFTNEGVMAKGVTVIDGFTQYFGEDGFQAKDKLVTFKGKTYYFDAHTGNGIKDTWR | |
| | NINGKWYYFDANGVAATGAQVINGQKLYFNEDGSQVKGGVVKNADGTYSKYKEGFGELVT | |
| | NEFFTTDGNVWYYAGANGKTVTGAQVINGQHLYFNADGSQVKGGVVKNADGTYSKYNAST | |
| | GERLTNEFFTTGDNNWYYIGANGKSVTGEVKIGDDTYFFAKDGKQVKGQTVSAGNGRISY | |
| | YYGDSGKRAVSTWIEIQPGVYVYFDENGLAYPPRVLN | |
| 7 | MVDGKYYYYDQDGNVKKNFAVSVGEKIYYFDETGAYKDTSKVEADKSGSDISKEETTFAA | Streptococcus |
| | NNRAYSTSAENFEAIDNYLTADSWYRPKSILKDGKTWTESSKDDFRPLLMAWWPDTETKR | downei |
| | NYVNYMNKVVGIDKTYTAETSQADLTAAAELVQARIEQKITTEQNTKWLREAISAFVKTQ | |
| | PQWNGESEKPYDDHLQNGALKFDNQSDLTPDTQSNYRLLNRTPTNQTGSLDSRFTYNAND | |
| | PLGGYELLLANDVDNSNPIVQAEQLNWLHYLLNFGTIYAKDADANFDSIRVDAVDNVDAD | |
| | LLQISSDYLKAAYGIDKNNKNANNHVSIVEAWSDNDTPYLHDDGDNLMNMDNKFRLSMLW | |
| | SLAKPLDKRSGLNPLIHNSLVDREVDDREVETVPSYSFARAHDSEVQDLIRDIIKAEINP | |
| | NAFGYSFTQDEIDQAFKIYNEDLKKTDKKYTHYNVPLSYTLLLTNKGSIPRVYYGDMFTD | |
| | DGQYMANKTVNYDAIESLLKARMKYVAGGQAMQNYQIGNGEILTSVRYGKGALKQSDKGD | |
| | ATTRTSGVGVVMGNQPNFSLDGKVVALNMGAAHANQEYRALMVSTKDGVATYATDADASK | |
| | AGLVKRTDENGYLYFLNDDLKGVANPQVSGFLQVWVPVGAADDQDIRVAASDTASTDGKS | |
| | LHQDAAMDSRVMFEGFSNFQSFATKEEEYTNVVIANNVDKFVSWGITDFEMAPQYVSSTD | |
| | GQFLDSVIQNGYAFTDRYDLGMSKANKYGTADQLVKAIKALHAKGLKVMADWVPDQHYTF | |
| | PKQEVVTVTRTDKFGKPIAGSQINHSLYVTDTKSSGDDYQAKYGGAFLDELKEKYPELFT | |
| | KKQISTGQAIDPSVKIKQWSAKYFNGSNILGRGADYVLSDQASNKYLNVTVSKLFLPKTL | |
| | LGQVVESGIRFDGTGYVYNSSTTGEKVTDSFITEAGNLYYFGQDGYMVTGAQNIKGSNYY | |
| | FLANGAALRNTVYTDAQGQNHYYGNDGKRYENGYQQFGNDSWRYFKNGVMALGLTTVDGH | |
| | VQYFDKDGVQAKDKIIVTRDGKVRYFDQHNGNAVTNTFVADKTGHWYYLGKDGVAVTGAQ | |
| | TVGKQHLYFEANGQQVKGDFVTAKDGKLYFYDVDSGDMWTNTFIEDKAGNWFYLGKDGAA | |
| | VTGAQTIKGQKLYFKANGQQVKGDIVKDADGKIRYYDAQTGEQVFNKSVSVNGKTYYFGS | |
| | DGTAQTQANPKGQTFKDGSGVLRFYNLEGQYVSGSGWYETAEHEWVYVKSGKVLTGAQTI | |
| | GNQRVYFKDNGHQVKGQLVTGNDGKLRYYDANSGDQAFNKSVTVNGKTYYFGSDGTAQTQ | |
| | ANPKGQTFKDGSGVLRFYNLEGQYVSGSGWYKNAQGQWLYVKDGKVLTGLQTVGNQKVYF | |
| | DKNGIQAKGKAVRTSDGKVRYFDENSGSMITNQWKFVYGQYYYFGSDGAAVYRGWN | |
| 8 | MIDGKYYYYDNNGKVRTNFTLIADGKILHFDETGAYTDTSIDTVNKDIVTTRSNLYKKYN | Streptococcus |
| | QVYDRSAQSFEHVDHYLTAESWYRPKYILKDGKTWTQSTEKDFRPLLMTWWPSQETQRQY | mutans |
| | VNFMNAQLGINKTYDDTSNQLQLNIAAATIQAKIEAKITTLKNTDWLRQTISAFVKTQSA | |
| | WNSDSEKPFDDHLQNGAVLYDNEGKLTPYANSNYRILNRTPTNQTGKKDPRYTADNTIGG | |
| | YEFLLANDVDNSNPVVQAEQLNWLHFLMNFGNIYANDPDANFDSIRVDAVDNVDADLLQI | |
| | AGDYLKAAKGIHKNDKAANDHLSILEAWSDNDTPYLHDDGDNMINMDNKLRLSLLFSLAK | |
| | PLNQRSGMNPLITNSLVNRTDDNAETAAVPSYSFIRAHDSEVQDLIRDIIKAEINPNVVG | |
| | YSFTMEEIKKAFEIYNKDLLATEKKYTHYNTALSYALLLTNKSSVPRVYYGDMFTDDGQY | |
| | MAHKTINYEAIETLLKARIKYVSGGQAMRNQQVGNSEIITSVRYGKGALKAMDTGDRTTR | |
| | TSGVAVIEGNNPSLRLKASDRVVVNMGAAHKNQAYRPLLLTTDNGIKAYHSDQEAAGLVR | |
| | YTNDRGELIFTAADIKGYANPQVSGYLGVWVPVGAAADQDVRVAASTAPSTDGKSVHQNA | |
| | ALDSRVMFEGFSNFQAFATKKEEYTNVVIAKNVDKFAEWGVTDFEMAPQYVSSTDGSFLD | |
| | SVIQNGYAFTDRYDLGISKPNKYGTADDLVKAIKALHSKGIKVMADWVPDQMYALPEKEV | |
| | VTATRVDKYGTPVAGSQIKNTLYVVDGKSSGKDQQAKYGGAFLEELQAKYPELFARKQIS | |
| | TGVPMDPSVKIKQWSAKYFNGTNILGRGAGYVLKDQATNTYFNISDNKEINFLPKTLLNQ | |
| | DSQVGFSYDGKGYVYYSTSGYQAKNTFISEGDKWYYFDNNGYMVTGAQSINGVNYYFLPN | |
| | GLQLRDAILKNEDGTYAYYGNDGRRYENGYYQFMSGVWRHFNNGEMSVGLTVIDGQVQVF | |
| | DEMGYQAKGKFVTTADGKIRYFDKQSGNMYRNRFIENEEGKWLYLGEDGAAVTGSQTING | |
| | QHLYFRANGVQVKGEFVTDRHGRISYYDGNSGDQIRNRFVRNAQGQWFYFDNNGYAVTGA | |
| | RTINGQHLYFRANGVQVKGEFVTDRHGRISYYDGNSGDQIRNRFVRNAQGQWFYFDNNGY | |
| | AVTGARTINGQHLYFRANGVQVKGEFVTDRYGRISYYDGNSGDQIRNRFVRNAQGQWFYF | |
| | DNNGYAVTGARTINGQHLYFRANGVQVKGEFVTDRYGRISYYDANSGERVRIN | |
| 9 | MVDGKYYYYDADGNVKKNFAVSVGDAIFYFDETGAYKDTSKVDADKTSSSVNQTTETFAA | Streptococcus |
| | NNRAYSTAAENFEAIDNYLTADSWYRPKSILKDGTTWTESTKDDFRPLLMAWWPDTETKR | dentirousetti |
| | NYVNYMNKVVGIDKTYTAETSQADLTAAAELVQARIEQKITSEKNTKWLREAISAFVKTQ | |
| | PQWNGESEKPYDDHLQNGALKFDNETSLTPDTQSGYRILNRTPTNQTGSLDPRFTFNQND | |
| | PLGGYEYLLANDVDNSNPVVQAESLNWLHYLLNFGSIYANDPEANFDSIRVDAVDNVDAD | |
| | LLQISSDYLKSAYKIDKNNKNANDHVSIVEAWSDNDTPYLNDDGDNLMNMDNKFRLSMLW | |
| | SLAKPTNVRSGLNPLIHNSVVDREVDDREVEATPNYSFARAHDSEVQDLIRDIIKAEINP | |
| | NSFGYSFTQEEIDQAFKIYNEDLKKTNKKYTHYNVPLSYTLLLTNKGSIPRIYYGDMFTD | |
| | DGQYMANKTVNYDAIESLLKARMKYVSGGQAMQNYNIGNGEILTSVRYGKGALKQSDKGD | |
| | KTTRTSGIGVVMGNQSNFSLEGKVVALNMGATHTKQKYRALMVSTETGVAIYNSDEEAEA | |
| | AGLIKTTDENGYLYFLNDDLKGVANPQVSGFLQVWVPVGAPADQDIRVAATDAASTDGKS | |
| | LHQDAALDSRVMFEGFSNFQSFATKEEEYTNVVIAKNVDKFVSWGITDFEMAPQYVSSTD | |
| | GTFLDSVIQNGYAFTDRYDLGMSKANKYGTADQLVAAIKALHAKGLRVMADWVPDQMYTF | |
| | PKKEVVTVTRTDKFGNPVAGSQINHTLYVTDTKGSGDDYQAKYGGAFLDELKEKYPELFT | |
| | KKQISTGQAIDPSVKIKQWSAKYFNGSNILGRGANYVLSDQASNKYFNVAEGKVFLPAAM | |
| | LGKVVESGIRFDGKGYIYNSSTTGEQVKDSFITEAGNLYYFGKDGYMVMGAQNIQGANYY | |
| | FLANGAALRNSILTDQDGKSHYYANDGKRYENGYYQFGNDSWRYFENGVMAVGLTRVAGH | |
| | DQYFDKDGIQAKNKIIVTRDGKVRYFDEHNGNAATNTFISDQAGHWYYLGKDGVAVTGAQ | |
| | TVGKQHLYFEANGQQVKGDFVTAKDGKLYFLDGDSGDMWTDTFVQDKAGHWFYLGKDGAA | |
| | VTGAQTVRGQKLYFKANGQQVKGDIVKGADGKIRYYDANSGDQVYNRTVKGSDGKTYIIG | |
| | NDGVAITQTIAKGQTIKDGSVLRFYSMEGQLVTGSGWYSNAKGQWLYVKNGQVLTGLQTV | |
| | GSQRVYFDANGIQAKGKAVRTSDGKLRYFDANSGSMITNQWKEVNGQYYYFDNNGVAIYR | |
| | GWN | |
| 10 | MIDGKNYYVQDDGTVKKNFAVELNGRILYFDAETGALVDSNEYQFQQGTSSLNNEFSQKN | Streptococcus |
| | AFYGTTDKDIETVDGYLTADSWYRPKFILKDGKTWTASTETDLRPLLMAWWPDKRTQINY | oralis |
| | LNYMNQQGLGAGAFENKVEQALLTGASQQVQRKIEEKIGKEGDTKWLRTLMGAFVKTQPN | |
| | WNIKTESETTGTKKDHLQGGALLYTNNEKSPHADSKFRLLNRTPTSQTGTPKYFIDKSNG | |
| | GYEFLLANDFDNSNPAVQAEQLNWLHYMMNFGSIVANDPTANFDGVRVDAVDNVNADLLQ | |
| | IASDYFKSRYKVGESEEEAIKHLSILEAWSDNDPDYNKDTKGAQLAIDNKLRLSLLYSFM | |
| | RNLSIRSGVEPTITNSLNDRSSEKKNGERMANYIFVRAHDSEVQTVIADIIRENINPNTD | |
| | GLTFTMDELKQAFKIYNEDMRKADKKYTQFNIPTAHALMLSNKDSITRVYYGDLYTDDGQ | |
| | YMEKKSPYHDAIDALLRARIKYVAGGQDMKVTYMGVPREADKWSYNGILTSVRYGTGANE | |
| | ATDEGTAETRTQGMAVIASNNPNLKLNEWDKLQVNMGAAHKNQYYRPVLLTTKDGISRYL | |
| | TDEEVPQSLWKKTDANGILTFDMNDIAGYSNVQVSGYLAVWVPVGAKADQDARTTASKKK | |
| | NASGQVYESSAALDSQLIYEGFSNFQDFATRDDQYTNKVIAKNVNLFKEWGVTSFELPPQ | |
| | YVSSQDGTFLDSIIQNGYAFEDRYDMAMSKNNKYGSLKDLLNALRALHSVNIQAIADWVP | |
| | DQIYNLPGKEVVTATRVNNYGTYREGAEIKEKLYVANSKTNETDFQGKYGGAFLDELKAK | |
| | YPEIFERVQISNGQKMTTDEKITKWSAKYFNGTNILGRGAYYVLKDWASNDYLTNRNGEI | |
| | VLPKQLVNKNSYTGFVSDANGTKFYSTSGYOAKNSFIQDENGNWYYFDKRGYLVTGAHEI | |
| | DGKHVYFLKNGIQLRDSIREDENGNQYYYDQTGAQVLNRYYTTDGQNWRYFDAKGVMARG | |
| | LVKIGDGQQFFDENGYQVKGKIVSAKDGKLRYFDKDSGNAVINRFAQGDNPSDWYYFGVE | |
| | FAKLTGLQKIGQQTLYFDQDGKQVKGKIVTLSDKSIRYFDANSGEMAVGKFAEGAKNEWY | |
| | YFDKTGKAVTGLQKIGKQTLYFDQDGKQVKGKVVTLADKSIRYEDADSGEMAVGKFAEGA | |
| | KNEWYYFDQTGKAVTGLQKIDKQTLYFDQDGKQVKGKIVTLSDKSIRYFDANSGEMATNK | |
| | FVEGSQNEWYYFDQAGKAVTGLQQVGQQTLYFTQDGKQVKGKVVDVNGVSRYFDANSGDM | |
| | ARSKWIQLEDGSWMYFDRDGRGQNFGRN | |
| 11 | MIDGKKYYVQDDGTVKKNFAVELNGKILYFDAETGALIDSAEYQFQQGTSSLNNEFTQKN | Streptococcus |
| | AFYGTTDKDVETIDGYLTADSWYRPKFILKDGKTWTASTEIDLRPLLMAWWPDKQTQVSY | sanguinis |
| | LNYMNQQGLGAGAFENKVEQAILTGASQQVQRKIEERIGKEGDTKWLRTLMGAFVKTQPN | |
| | WNIKTESETTGTNKDHLQGGALLYSNSDKTSHANSKYRILNRTPTNQTGTPKYFIDKSNG | |
| | GYEFLLANDFDNSNPAVQAEQLNWLHFMMNFGSIVANDPTANFDGVRVDAVDNVNADLLQ | |
| | IASDYFKSRYKVGESEEEAIKHLSILEAWSDNDPDYNKDTKGAQLPIDNKLRLSLLYSFM | |
| | RKLSIRSGVEPTITNSLNDRSTEKKNGERMANYIFVRAHDSEVQTVIADIIRENINPNTD | |
| | GLTFTMDELKQAFKIYNEDMRKADKKYTQFNIPTAHALMLSNKDSITRVYYGDLYTDDGQ | |
| | YMEKKSPYHDAIDALLRARIKYVAGGQDMKVTYMGVPREADKWSYNGILTSVRYGTGANE | |
| | ATDEGTAETRTQGMAVIASNNPNLKLNEWDKLQVNMGAAHKNQYYRPVLLTTKDGISRYL | |
| | TDEEVPQSLWKKTDANGILTFDMNDIAGYSNVQVSGYLAVWVPVGAKADQDARVTASKKK | |
| | NASGQVYESSAALDSQLIYEGFSNFQDFATRDDQYTNKVIAKNVNLFKEWGVTSFELPPQ | |
| | YVSSQDGTFLDSIIQNGYAFEDRYDMAMSKNNKYGSLNDLLNALRALHSVNIQAIADWVP | |
| | DQIYNLPGKEVVTATRVNNYGTYREGSEIKENLYVANTKTNGTDYQGKYGGAFLDELKAK | |
| | YPEIFERVQISNGQKMTTDEKITKWSAKHFNGTNILGRGAYYVLKDWASNEYLNNKNGEM | |
| | VLPKQLVNKNAYTGFVSDASGTKYYSTSGYQARNSFIQDENGNWYYFNNRGYLVTGAQEI | |
| | DGKQLYFLKNGIQLRDSLREDENGNQYYYDKTGAQVLNRYYTTDGQNWRYFDVKGVMARG | |
| | LVTMGGNQQFFDQNGYQVKGKIARAKDGKLRYFDKDSGNAAANRFAQGDNPSDWYYFGAD | |
| | GVAVTGLQKVGQQTLYFDQDGKQVKGKVVTLADKSIRYFDANSGEMAVNKFVEGAKNVWY | |
| | YFDQAGKAVTGLQTINKQVLYFDQDGKQVKGKVVTLADKSIRYFDANSGEMAVGKFAEGA | |
| | KNEWYYFDQAGKAVTGLQKIGQQTLYEDQNGKQVKGKVVTLADKSIRYFDANSGEMASNK | |
| | FVEGAKNEWYYFDQAGKAVTGLQQIGQQTLYEDQNGKQVKGKIVYVNGANRYFDANSGEM | |
| | ARNKWIQLEDGSWMYFDRNGRGRRFGWN | |
| 12 | MIDGKYYYVNEDGSHKENFAITVNGQLLYFGKDGALTSSSTYSFTQGTTNIVDGFSINNR | Unknown |
| | AYDSSEASFELIDGYLTADSWYRPASIIKDGVTWQASTAEDFRPLLMAWWPNVDTQVNYL | streptococcus |
| | NYMSKVFNLDAKYSSTDKQETLKVAAKDIQIKIEQKIQAEKSTQWLRETISAFVKTQPQW | species |
| | NKETENYSKGGGEDHLQGGALLYVNDSRTPWANSNYRLLNRTATNQTGTIDKSILDEQSD | |
| | PNHMGGFDFLLANDVDLSNPVVQAEQLNQIHYLMNWGSIVMGDKDANFDGIRVDAVDNVD | |
| | ADMLQLYTNYFREYYGVNKSEANALAHISVLEAWSLNDNHYNDKTDVAALAMENKQRLAL | |
| | LFSLAKPIKERTPAVSPLYNNTFNTTQRDEKTDWINKDGSKAYNEDGTVKKSTIGKYNEK | |
| | YGDASGNYVFIRAHDNNVQDIIAEIIKKEINEKSDGFTITDSEMKRAFEIYNKDMLSNDK | |
| | KYTLNNIPAAYAVMLQNMETITRVYYGDLYTDDGNYMEAKSPYYDTIVNLMKSRIKYVSG | |
| | GQAQRSYWLPTDGKMDKSDVELYRTNEVYTSVRYGKDIMTADDTQGSKYSRTSGQVTLVV | |
| | NNPKLTLDQSAKLNVVMGKIHANQKYRALIVGTPNGIKNFTSDAEAIAAGYVKETDGNGV | |
| | LTFGANDIKGYETFDMSGFVAVWVPVGASDDQDIRVAASTAAKKEGELTLKATEAYDSQL | |
| | IYEGFSNFQTIPDGSDPSVYTNRKIAENVDLFKSWGVTSFEMAPQFVSADDGTFLDSVIQ | |
| | NGYAFADRYDLAMSKNNKYGSKEDLRNALKALHKAGIQAIADWVPDQIYQLPGKEVVTAT | |
| | RTDGAGRKISDAIIDHSLYVANSKSSGKDYQAKYGGEFLAELKAKYPEMEKVNMISTGKP | |
| | IDDSVKLKQWKAEYFNGTNVLDRGVGYVLSDEATGKYFTVTKEGNFIPLQLKGNKKVITG | |
| | FSSDGKGITYFGTSGNQAKSAFVTFNGNTYYFDARGHMVTNGEYSPNGKDVYRFLPNGIM | |
| | LSNAFYVDGNGNTYLYNSKGQMYKGGYSKFDVTETKDGKESKVVKFRYFTNEGVMAKGVT | |
| | VVDGFTQYFNEDGIQSKDELVTYNGKTYYFEAHTGNAIKNTWRNIKGKWYHFDANGVAAT | |
| | GAQVINGQHLYFNEDGSQVKGSIVKNADGTFSKYKDSSGDLVVNEFFTTGDNVWYYAGAN | |
| | GKTVTGAQVINGQHLFFKEDGSQVKGDFVKNSDGTYSKYDAASGERLTNEFFTTGDNHWY | |
| | YIGANGKTVTGEVKIGDDTYFFAKDGKQLKGQIVTTRSGRISYYFGDSGKKAISTWVEIQ | |
| | PGVFVFFDKNGLAYPPENMN | |
| 13 | MIDGKYYYVNKDGSHKENFAITVNGQLLYFGKDGALTSSSTYSFTQGTTNIVDGFSKNNR | Streptococcus |
| | AYDSSEASFELIDGYLTADSWYRPVSIIKDGVTWQASTKEDFRPLLMAWWPNVDTQVNYL | salivarius |
| | NYMSKVFNLDAKYTSTDKQVDLNRAAKDIQVKIEQKIQAEKSTQWLREAISAFVKTQPQW | |
| | NKETENFSKGGGEDHLQGGALLYVNDPRTPWANSNYRLLNRTATNQTGTIDKSVLDEQSD | |
| | PNHMGGFDFLLANDVDTSNPVVQAEQLNQIHYLMNWGSIVMGDKDANFDGIRVDAVDNVD | |
| | ADMLQLYTNYFREYYGVNKSEANALAHISVLEAWSLNDNHYNDKTDGAALAMENKQRLAL | |
| | LFSLAKPIKERTPAVSPLYNNTFNTTQRDEKTDWINKDGSKAYNEDGTVKQSTIGKYNEK | |
| | YGDASGNYVFIRAHDNNVQDIIAEIIKKEINPKSDGFTITDAEMKKAFEIYNKDMLSSDK | |
| | KYTLNNIPAAYAVMLQNMETITRVYYGDLYTDDGHYMETKSPYYDTIVNLMKNRIKYVSG | |
| | GQAQRSYWLPTDGKMDKSDVELYRTNEVYTSVRYGKEIMTADDTQGSKYSRTSGQVTLVV | |
| | NNPKLSLDKSAKLDVEMGKIHANQKYRALIVGTPNGIKNFTSDAEAIAAGYVKETDGNGV | |
| | LTFGANDIKGYETFDMSGFVAVWVPVGASDDQDIRVAASTAAKKEGELTLKATEAYDSQL | |
| | IYEGESNFQTIPDGSDPSVYTNRKIAEDWDLFKSWGVTSFEMAPQFVSADDGTFLDSVIQ | |
| | NGYAFADRYDLAMSKNNKYGSKEDLRNALKALHKAGIQAIADWVPDQIYQLPGKEVVTAT | |
| | RTDGAGRKISDAIIDHSLYVANSKSSGKDYQAKYGGEFLAELKAKYPEMFKVNMISTGKP | |
| | IDDSVKLKQWKAEYFNGTNVLDRGVGYVLSDEATGKYFTVTKEGNFIPLQLKGNEKVITG | |
| | FSSDGKGITYEGTSGNQAKSAFVTFNGNTYYFDARGHMVTNGEYSPNGKDVYRFLPNGIM | |
| | LSNAFYVDGNGNTYLYNSKGQMYKGGYSKFDVTETKDGKESKVVKFRYFTNEGVMAKGVT | |
| | VVDCFTQYFNEDGIQSKDELVTYNGKTYYFEAHTGNAIKNTWRNIKGKWYHFDANGVAAT | |
| | GAQVINGQHLYFNEDGSQVKGGVVKNADGTFSKYKDGSGDLVVNEFFTTGDNVWYYAGAN | |
| | GKTVTGAQVINGQHLFFKEDGSQVKGDFVKNSDGTYSKYDAASGERLTNEFFTTGDNHWY | |
| | YIGANGKTVTGEVKIGDDTYFFAKDGKQLKGQIVTTRSGRISYYFGDSGKKAISTWVEIQ | |
| | PGVFVFFDKNGLAYPPENMN | |
| 14 | MTDGKYYYVNEDGSHKENFAITVNGQLLYFGKDGALTSSSTHSFTPGTTNIVDGFSINNR | Streptococcus |
| | AYDSSEASFELINGYLTADSWYRPV$IIKDGVTWQASTAEDFRPLLMAWWPNVDTQVNYL | salivarius |
| | NYMSKVFNLEAKYTSTDKQADLNRAAKDIQVKIEQKIQAEKSTQWLRETISAFVKTQPQW | |
| | NKETENYSKGGGEDHLQGGALLYVNDSRTPWANSNYRLLNRTATNQTGTINKSVLDEQSD | |
| | PNHMGGFDFLLANDVDLSNPVVQAEQLNQIHYLMNWGSIVMGDKDANFDGIRVDAVDNVN | |
| | ADMLQLYTNYFREYYGVNKSEAQALAHISVLEAWSLNDNHYNDKTDGAALAMENKQRLAL | |
| | LFSLAKPIKDRTPAVSPLYNNTFNTTQRDFKTDWINKDGSTAYNEDGTAKQSTIGKYNEK | |
| | YGDASGNYVFIRAHDNNVQDIIAEIIKKEINKKSDGFTISDSEMKQAFEIYNKDMLSSNK | |
| | KYTLNNIPAAYAVMLQNMETITRVYYGDLYTDDGHYMETKSPYHDTIVNLMKNRIKYVSG | |
| | GQAQRSYWLPTDGKMDNSDVELYRTSEVYTSVRYGKDIMTADDTEGSKYSRTSGQVTLVV | |
| | NNPKLTLHESAKLNVEMGKIHANQKYRALIVGTADGIKNFTSDAEAIAAGYVKETDSNGV | |
| | LTFGANDIKGYETFDMSGFVAVWVPVGASDDQDIRVAPSTEAKKEGELTLKATEAYDSQL | |
| | IYEGFSNFQTIPDGSDPSVYTNRKIAENVDLFKSWGVTSFEMAPQFVSADDGTFLDSVIQ | |
| | NGYAFADRYDLAMSKNNKYGSKEDLRDALKALHKAGIQAIADWVPDQIYQLPGKEVVTAT | |
| | RTDGAGRKIADAIIDHSLYVANSKSSGRDYQAQYGGEFLAELKAKYPKMFTENMISTGKP | |
| | IDDSVKLKQWKAKYFNGTNVLDRGVGYVLSDEATGKYFTVTKEGNFIPLQLTGNEKAVTG | |
| | FSNDGKGITYFGTSGNQAKSAFVTFNGNTYYFDARGHMVTNGEYSPNGKDVYRFLPNGIM | |
| | LSNAFYVDANGNTYLYNYKGQMYKGGYTKFDVTETDKDGNESKVVKFRYFTNEGVMAKGL | |
| | TVIDGSTQYFGEDGFQTKDKLATYKGKTYYFEAHTGNAIKNTWRNIDGKWYHFDENGVAA | |
| | TGAQVINGQKLYFNEDGSQVKGGVVKNADGTYSKYKEGSGELVTNEFFTTDGNVWYYAGA | |
| | DGKTVTGAQVINGQHLYFKEDGSQVKGGVVKNADGTYSKYDAATGERLTNEFFTTGDNNW | |
| | YYIGSNGKTVTGEVKIGADTYYFAKDGKQVKGQTVTAGNGRISYYYGDSGKKAISTWIEI | |
| | QPGIYVYFDKTGIAYPPRVLN | |
| 15 | MIDGKYYYVNEDGSHKENFAITVNGQLLYFGKDGALTSSSTYSFTPGTTNIVDGFSINNR | Streptococcus |
| | AYDSSEASFELIDGYLTADSWYRPASIIKDGVTWQASTAEDFRPLLMAWWPNVDTQVNYL | salivarius |
| | NYMSKVFNLDAKYTSTDKQETLNVAAKDIQVKIEQKIQAEKSTQWLRETISAFVKTQPQW | |
| | NKETENYSKGGGEDHLQGGALLYVNDSRTPWANSNYRLLNHTATNQKGTIDKSVLDEQSD | |
| | PNHMGGFDFLLANDVDLSNPVVQAEQLNQIHYLMNWGSIVMGDKDANFDGIRVDAVDNVD | |
| | ADMLQLYTNYFREYYGVNKSEANALAHISVLEAWSLNDNHYNDKTDGAALAMENKQRLAL | |
| | LFSLAKPIKERTPAVSPLYNNTFNTTQRDEKTDWINKDGSKAYNEDGTVKQSTIGKYNEK | |
| | YGDASGNYVFIRAHDNNVQDIIAEIIKKEINPKSDGFTITDAEMKKAFEIYNKDMLSSDK | |
| | KYTLNNIPAAYAVMLQNMETITRVYYGDLYTDNGNYMETKSPYYDTIVNLMKNRIKYVSG | |
| | GQAQRSYWLPTDGKMDNSDVELYRTNEVYASVRYGKDIMTADDTEGSKYSRTSGQVTLVA | |
| | NNPKLTLDQSAKLKVEMGKIHANQKYRALIVGTADGIKNFTSDADAIAAGYVKETDSNGV | |
| | LTFGANDIKGYETFDMSGFVAVWVPVGASDDQDIRVAPSTEAKKEGELTLKATEAYDSQL | |
| | IYEGFSNFQTIPDGSDPSVYTNRKIAENVDLFKSWGVTSFEMAPQFVSADDGTFLDSVIQ | |
| | NGYAFADRYDLAMSKNNKYGSKEDLRDALKALHKAGIQAIADWVPDQIYQLPGKEVVTAT | |
| | RTDGAGRKIADAIIDHSLYVANTKSSGKDYQAKYGGEFLAELKAKYPEMFKVNMISTGKP | |
| | IDDSVKLKQWKAEYFNGTNVLERGVGYVLSDEATGKYFTVTKDGNFIPLQLTGNEKVVTG | |
| | FSNDGKGITYFGTSGTQAKSAFVTFNGNTYYFDARGHMVTNGEYSPNGKDVYRFLPNGIM | |
| | LSNAFYVDANGNTYLYNSKGQMYKGGYTKFDVTETDKDGKESKVVKFRYFTNEGVMAKGV | |
| | TVIDGFTQYFGEDGFQAKDKLVTFKGKTYYFDAHTGNAIKNTWRNIDGKWYHFDANGVAA | |
| | TGAQVINGQKLYFNEDGSQVKGGVVKNADGTYSKYKEGSGELVTNEFFTTDGNVWYYAGA | |
| | NGKTVTGAQVINGQHLYFNADGSQVKGGVVKNADGTYSKYDAATGERLTNEFFTTGDNNW | |
| | YYIGANGKTVTGEVKIGDDTYYFAKDGKQVKGQTVSAGNGRISYYYGDSGKRAVSTWVEI | |
| | QPGVYVYFDKNGLAYPPRVLN | |

### PREFERRED EMBODIMENTS OF THE INVENTION

In accordance with an aspect of the present invention, a method is provided for generating glucose polymers in a dairy product using a glucosyl transferase to provide increased texture. The method provides high, robust, and smooth texture from the formed glucose polymers. As set forth above, in the context of the present invention, texture means thickness and/or mouthfeel. As discussed above, there is wide spread use in the yogurt industry of starch to provide texture in yogurt. The methods of the present invention surprisingly provide an alternative to starch and other stabilizers for adding texture to yogurt.

In accordance with an aspect of the present invention, added sucrose is converted into glucose polymers and fructose. While the glucose polymers increase the texture of the yogurt, the fructose gives the yogurt a fructose sweetness. Fructose enhances palatability and taste of the yogurt in addition to the improved texture.

In some jurisdictions, components such as enzyme potentially require labeling of the final product as having the component as an ingredient. In an aspect of the present invention, the GTF would be considered a processing aid because the milk maybe heated (including pasteurization), inactivating the GTF. Surprisingly, it has been found that the increased texture provided by the instant invention is not destroyed by heating, even up to 95° C. for 6 minutes.

In another aspect of the present invention, it was found that the glucose polymers produced in milk at a neutral pH may have a non-uniform or non-homogenous appearance. After inoculation with culture during the fermentation process, the pH drops and it was found that the glucose polymers present during fermentation have a more homogenous, shiny look.

As mentioned above, stabilizers are frequently added to yogurt to increase texture. In addition to increasing expense because of the cost of the ingredient, added stabilizers such as starch require special handling procedures when the yogurt is poured into smaller containers for distribution to consumers. Yogurt manufacturers must typically cool yogurt to 8° C. before it can be shipped out for distribution to stores. While it is possible to quickly batch chill yogurt using cooling plates, this is not possible for yogurt having stabilizer. If yogurt with stabilizer is batch chilled to 8° C. before filling into individual containers for consumer purchase, the texture provided by the stabilizer will be destroyed during the filling process by shear forces. Texture lost in this way cannot be restored, defeating the entire point of adding stabilizer to begin with.

Stabilizer containing yogurt must be filled into containers at 20 to 25° C. Once the yogurt with stabilizer is filled into containers, it can be cooled to approximately 8° C. and shipped. However, cooling in this way is slower and causes delays in shipping and added expense in terms of providing a cooling facility.

In accordance with an aspect of the present invention, it was discovered that the yogurt containing the produced glucose polymers may be cooled to 5° C. before filling. This feature allows for substantial costs savings.

In another aspect of the instant invention, the yogurts containing the produced glucose polymers may be combined with stabilizers such as starch or pectin to provide long shelf life, highly stable, increased texture yogurt. Stabilizers may also be used to prevent sedimentation of protein caused by heating of the yogurt at low pH.

The protein and fat content of yogurt can be modified for cost and/or perceived health reasons. For example, fat provides texture and a desirable taste to yogurt. However, for health reasons consumers may prefer low fat or even non-fat yogurt. The glucose polymers produced in accordance with the instant invention can replace the texture lost by reducing or eliminating fat. Increasing yogurt protein content is also a way of increasing texture. However, there is an expense associated with boosting yogurt protein content. In accordance with the present invention, it has been discovered that the glucose polymers of the instant invention can provide texture in place of or in addition to the added protein.

In accordance with an aspect of the present invention, a method is presented for making a yogurt product having improved texture, improved texture being increased thickness and/or mouthfeel, having the steps of: providing milk; adding sucrose to the milk to form sweetened milk; contacting the sweetened milk with a glucosyl transferase to form an insoluble glucose polymer; inoculating with a starter culture; and fermenting to provide the yogurt product having improved texture which is increased thickness and/or increased mouthfeel.

Preferably, the milk is cow's milk. Preferably, the milk is selected from the group consisting of raw milk, pre-pasteurized milk, whole milk, skim milk, reconstituted milk, lactase treated milk, reduced lactose milk, lactose free milk and condensed milk. In other preferred embodiments, the milk is raw milk.

Preferably, the method has the additional steps of homogenizing and pasteurizing the milk. In a preferred aspect of the instant invention, the step of contacting with glucosyl transferase is performed after the steps of homogenizing and pasteurizing. In yet another preferred embodiment, the step of contacting with glucosyl transferase is performed before the steps of homogenizing and pasteurizing.

Preferably, the sucrose is added to constitute about 0.1 to 12% (w/w). More preferably, the sucrose is added to constitute about 2 to 8% (w/w). In still more preferred embodiments, the sucrose is added to constitute about 4 to 6% (w/w).

Preferably, the glucosyl transferase is an enzyme which has at least 70% sequence identity to an enzyme selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO: 12), GTF2919 (SEQ ID NO: 13), GTF2678 (SEQ ID NO; 14), and GTF3929 (SEQ ID NO: 15). More preferably, the glucosyl transferase is an enzyme which has at least 80% sequence identity to an enzyme selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO: 12), GTF2919 (SEQ ID NO: 13), GTF2678 (SEQ ID NO; 14), and GTF3929 (SEQ ID NO: 15). Still more preferably, the glucosyl transferase is an enzyme which has at least 90% sequence identity to an enzyme selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO: 12), GTF2919 (SEQ ID NO: 13), GTF2678 (SEQ ID NO; 14), and GTF3929 (SEQ ID NO: 15). In yet more preferred embodiments, the glucosyl transferase is an enzyme which has at least 95% sequence identity to GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO: 12), GTF2919 (SEQ ID NO: 13), GTF2678 (SEQ ID NO, 14), and GTF3929 (SEQ ID NO: 15). In still more preferred embodiments, the glucosyl transferase is selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO: 12), GTF2919 (SEQ ID NO: 13), GTF2678 (SEQ ID NO; 14), and GTF3929 (SEQ ID NO: 15). Still more preferably the glucosyl transferase is GTFJ (SEQ ID NO: 1).

Preferably, the glucosyl transferase is present in the milk in an amount from about 0.005 mg per 100 ml milk to 15 mg per 100 ml milk. More preferably, the glucosyltransferase is present in an amount from about 0.03 mg per 100 ml milk to about 12.5 mg per 100 ml milk.

Preferably, the GTFJ is present in an amount from about 0.033 mg per 100 ml milk to about 12.5 mg per 100 ml milk. More preferably, the GTFJ is present in an amount from about 0.3 mg per 100 ml milk to about 5.0 mg per 100 ml milk.

In other preferred embodiments the glucosyl transferase is GTF300 (SEQ ID NO: 2). Preferably, the GTF300 is present in an amount from about 0.033 mg per 100 ml to about 12.5 mg per 100 ml milk. More preferably, the GTF300 is present in an amount from about 0.3 mg per 100 ml milk to about 5 mg per 100 ml milk.

Preferably, the increased texture is increased thickness. Preferably, the thickness is increased by 30% or more as compared with a control sample (no GTF enzyme). More preferably, the thickness is increased by 50% or more. Still more preferably, the thickness is increased by 70% or more. In yet more preferred embodiments, the thickness is increased by 90% or more. More preferably, the thickness is increased by 100% or more. Still more preferably, the thickness is increased by 110% or more. In the most preferred embodiments, the thickness is increased by 120% or more.

In other preferred embodiments, the increased texture is increased mouthfeel. Preferably the mouthfeel is increased by 30% or more as compared with a control sample (no GTF enzyme). More preferably, the mouthfeel is increased by 50% or more. Still more preferably, the mouthfeel is increased by 70% or more. In yet more preferred embodiments, the mouthfeel is increased by 90% or more. Still more preferably, the mouthfeel is increased by 100% or more. In yet more preferred embodiments, the mouthfeel is increased by 110% or more. In the most preferred embodiments, the mouthfeel is increased by 120% or more.

In accordance with an aspect of the present invention, the milk is low fat milk to provide a low fat yogurt. In a more preferred aspect of the present invention the milk is non-fat milk to provide a non-fat yogurt.

In another preferred aspect of the present invention, the protein content of the milk is adjusted to at least about 3% (w/w). More preferably, the protein content of the milk is adjusted to at least about 3.5%. Still more preferably, the protein content of the milk is adjusted to at least about 3.7% (w/w). In other preferred embodiment, the protein content of the milk is adjusted to at least about 3.8% (w/w). In still more preferred embodiments, the protein content of the milk is adjusted to at least about 3.9% (w/w). In still other preferred embodiments, the protein content of the milk is adjusted to at least about 4.0% (w/w).

In another aspect of the invention, the method includes the further steps of cooling the yogurt of to a temperature of between 5 and 10° C. to provide a chilled yogurt; and pouring the chilled yogurt into preformed containers. Preferably, the containers provide a single serving of yogurt. The preferred embodiments of this aspect of the present invention are as set forth above.

In another aspect of the present invention, a yogurt is presented which is made according to any of the above methods. Preferably, the yogurt has pectin.

In another aspect of the present invention, the milk comprises at least 4% lactose (w/w). Preferably, the milk comprises at least 4.5% lactose.

In another aspect of the present invention, a method is presented of making a reduced sugar food product having improved texture, the method having the steps of: providing a food matrix comprising sucrose and lactose; and contacting the food matrix with a glucosyl transferase to form an insoluble glucose polymer.

Preferably, the sucrose in the food matrix is from about 0.1 to about 12% (w/w). More preferably, the sucrose is from about 2 to about 8% (w/w). Still more preferably, the sucrose is from about 4 to about 6% (w/w).

Preferably, the lactose in the food matrix is from about 0.1 to about 12% (w/w). More preferably, the lactose is from about 2 to about 8% (w/w). Still more preferably, the lactose is from about 4 to about 6% (w/w)

Preferably, the improved texture in the food matrix is increased thickness and/or increased mouthfeel.

Preferably, the food matrix is a diary product, a beverage, a dough or bread, a confectionary, a fermented beverage, a dressing, a sauce, or a processed meat

Preferred glucosyltransferases are as set forth above.

Some embodiments herein are drawn to a method as presently disclosed, but in which at least one fructosyltransferase enzyme is used in place of, or in addition to, a glucosyltransferase as disclosed herein. A “fructosyltransferase” (or “fructansucrase”) herein refers to an enzyme capable of transferring fructose from sucrose substrate to a saccharide acceptor such as sucrose or a fructan, thereby producing glucose and a fructosylated saccharide product (e.g., a fructan such as 2,1-beta-fructan [inulin] or 2,6-beta-fructan [levan]). Given that a fructosyltransferase enzyme is used, the sucrose of a milk composition herein is converted to a fructan instead of glucan, and free glucose is produced instead of free fructose. Examples of fructosyltransferases herein are those as classified under Enzyme Commission (E.C.) Nos. 2.4.1.9 (e.g., inulosucrase) or 2.4.1.99 (e.g., levansucrase). Further examples include fructosyltransferases as disclosed in any of U.S. Pat. Nos. 5,952,205, 5,641,667 and 6,872,555, which are incorporated herein by reference. It is contemplated that production of fructan in dairy and other food products using a fructosyltransferase in situ allows for producing products with at least improved texture, dietary fiber, and/or prebiotic qualities. Alternatively, fructan can be directly added to dairy or other food products; such fructan can be a product of any of the foregoing fructosyltransferases, for example.

Some embodiments herein are drawn to a method as presently disclosed, but in which at least one dextransucrase enzyme is used in place of, or in addition to, a glucosyltransferase as disclosed herein. A dextransucrase herein refers to a type of glucosyltransferase enzyme capable of synthesizing dextran (e.g., water-soluble alpha-glucan comprising at least about 50%, 60%, 70%, 80%, 90%, 91%, 92%, 93%, 94%, 95%, 96%, 97%, 98%, 99%, or 100% alpha-1,6 glycosidic linkages) and fructose from sucrose, and is typically classified under EC 2.4.1.5. In some aspects, a dextransucrase can produce dextran having a weight-average molecular weight (Mw) of about, at least about, or no more than about, 25, 50, 100, 200, 500, or 850 million Daltons. A dextransucrase can, in some instances, produce dextran that comprises (i) about 87-93 wt % glucose linked at positions 1 and 6; (ii) about 0.1-1.2 wt % glucose linked at positions 1 and 3; (iii) about 0.1-0.7 wt % glucose linked at positions 1 and 4; (iv) about 7.7-8.6 wt % glucose linked at positions 1, 3 and 6; and (v) about 0.4-1.7 wt % glucose linked at: (a) positions 1, 2 and 6, or (b) positions 1, 4 and 6; and has an Mw of about 50-200 million Daltons and a z-average radius of gyration of about 200-280 nm. In some instances, a dextransucrase can produce dextran having a degree of polymerization (DP) or weight-average DP (DPw) of about, at least about, or no more than about, 10, 25, 50, 75, 100, 105, 110, 150, 200, 250, 300, 400, 500, 600, or 700. A dextran in some cases can comprise 1-50% alpha-1,2 branches (each branch typically is a single glucose unit), where such branches are added by the dextransucrase enzyme itself (one that further has alpha-1,2-branching activity) during dextran synthesis. Examples of dextransucrases with some of the foregoing capabilities (e.g., GTF 0768, GTF 8117, GTF 6831, GTF 5604, DSR-E) are as disclosed in any of U.S. Patent Appl. Publ. Nos. 2016/0122445, 2017/0145120, 2018/0282385, 2017/0218093 and 2010/0284972, which are all incorporated herein by reference. It is contemplated that production of dextran in dairy and other food products using a dextransucrase in situ allows for producing products with at least improved dietary fiber and/or prebiotic qualities. Alternatively, dextran can be directly added to dairy or other food products; such dextran can be a product of any of the foregoing dextransucrases, for example.

Some embodiments herein are drawn to a method as presently disclosed, but in which at least one variant (engineered) alpha-1,3-glucan-producing glucosyltransferase enzyme is used in place of, or in addition to, a glucosyltransferase as disclosed herein. Such a variant glucosyltransferase can produce alpha-1,3 glucan (e.g., water-insoluble alpha-glucan comprising at least about 50%, 60%, 70%, 80%, 90%, 91%, 92%, 93%, 94%, 95%, 96%, 97%, 98%, 99%, or 100% alpha-1,3 glycosidic linkages), and in some aspects the glucan product is of lower or higher molecular weight, and/or produced in higher yield, as compared to alpha-1,3-glucan produced by the enzyme's non-variant counterpart (e.g., parent enzyme). Examples of suitable parent enzymes are disclosed herein as GTF-J, GTF0874, GTF6855, GTF2379, GTF7527, GTF1724, GTF0544, GTF5926, GTF4297, GTF5618, GTF2765, GTF0427, GTF2919, GTF2678 and GTF3929; it is noted that GTF300 (SEQ ID NO:2) is a variant of GTF6855 (SEQ ID NO:4). A variant alpha-1,3-glucan-producing glucosyltransferase in some aspects can produce insoluble alpha-1,3-glucan with a DP or DPw of about, or less than about, 300, 280, 260, 240, 220, 200, 180, 160, 140, 120, 100, 80, 60, 50, 40, 30, 25, 20, 15, or 11, and/or can be as disclosed in U.S. patent application Ser. No. 16/295,423 (as originally filed), which is incorporated herein by reference. With respect to producing lower molecular weight alpha-1,3-glucan (e.g., DP or DPw<300), suitable substitution sites and examples of particular substitutions at these sites can include any of those as listed in Table 3 or 4 of U.S. patent application Ser. No. 16/295,423 that are associated with a decrease in DPw of insoluble alpha-1,3-glucan product by about, or at least about, 10%, 20%, 30%, 40%, 50%, 60%, 70%, 80%, or 85%, for example. With respect to producing higher molecular weight alpha-1,3-glucan, suitable substitution sites and examples of particular substitutions at these sites can include any of those as listed in Table 3, 4, or 5 of U.S. Patent Appl. Publ. No. 2019/0078062 (incorporated herein by reference) that are associated with an increase in DPw of insoluble alpha-1,3-glucan product by about, or at least about, 10%, 20%, 30%, 40%, 50%, or 60%, for example. With respect to producing alpha-1,3-glucan at a higher yield, suitable substitution sites and examples of particular substitutions at these sites can include any of those as listed in Table 3, 6, or 7 of U.S. Patent Appl. Publ. No. 2019/0078063 (incorporated herein by reference) that are associated with (i) a decrease in leucrose production by at least about 10%, 20%, 30%, 40%, 50%, 60%, 70%, 80%, 90%, or 95%, and/or (ii) an increase in glucan yield by at least about 10%, 20%, 30%, 40%, 50%, 60%, 70%, 80%, 90%, 100%, 110%, 120%, 130%, 140%, or 150%, for example. It is contemplated that production of alpha-1,3-glucan in dairy and other food products using a variant glucosyltransferase in situ allows for producing products with at least improved texture, dietary fiber, and/or prebiotic qualities. Alternatively, alpha-1,3-glucan as produced by (or producible from) a variant glucosyltransferase herein can be directly added to dairy or other food products.

In some aspects, alpha-1,3-glucan in the form of a dextran-alpha-1,3-glucan block copolymer can be directly added to milk or other dairy product. Examples of such block copolymers are disclosed in Int. Patent Appl. Publ. No. WO2017/079595 or U.S. Patent Appl. Publ. No. 2019/0185893, which are incorporated herein by reference. In some aspects, the dextran component of a dextran-alpha-1,3-glucan block copolymer (e.g., the dextran used to produce the block copolymer) comprises (i) about 87-93 wt % glucose linked at positions 1 and 6; (ii) about 0.1-1.2 wt % glucose linked at positions 1 and 3; (iii) about 0.1-0.7 wt % glucose linked at positions 1 and 4; (iv) about 7.7-8.6 wt % glucose linked at positions 1, 3 and 6; and (v) about 0.4-1.7 wt % glucose linked at: (a) positions 1, 2 and 6, or (b) positions 1, 4 and 6; and has an Mw of about 50-200 million Daltons and a z-average radius of gyration of about 200-280 nm. In some aspects, the dextran component of a dextran-alpha-1,3-glucan block copolymer can be produced using GTF 0768 as disclosed in U.S. Patent Appl. Publ. No. 2016/0122445. In some aspects, a dextran-alpha-1,3-glucan block copolymer comprises about, or at least about, 50, 55, 60, 65, 70, 75, 80, 85, or 90 wt % dextran. It is contemplated that direct addition of dextran-alpha-1,3-glucan block copolymer to dairy or other food products can provide at least improved texture, dietary fiber, and/or prebiotic qualities to products.

Some embodiments herein are drawn to a method of reducing the caloric content of, and/or increasing the dietary fiber content of, a food product or food precursor product. This method can comprise treating a saccharide-containing food product or food precursor product with first and second phosphorylase enzymes under suitable conditions, wherein the saccharide (present in the food product or food precursor product) is mammal (e.g., human)-digestible (i.e., caloric) and comprises glucose, and the first phosphorylase enzyme converts the mammal-digestible saccharide to products including alpha-glucose-1-phosphate (alpha-GIP), and the second phosphorylase enzyme reacts the alpha-G1P with a saccharide acceptor to produce a mammal-indigestible (i.e., non-caloric) saccharide. This method reduces the caloric content of the food product or food precursor product and/or increases the dietary fiber content of the food product or food precursor product. Features of this method can include, for example, any as disclosed in U.S. Patent Appl. Publ. No. 2017/0327857 or U.S. patent application Ser. No. 16/383,820 (as originally filed), which are incorporated herein by reference. A “phosphorylase” herein refers to a particular class of enzymes belonging to the glycosyl hydrolase 94 (GH94) family according to the CAZy (Carbohydrate-Active EnZymes) database (cazy.org website; see Cantarel et al., 2009, Nucleic Acids Res. 37:D233-238, incorporated herein by reference). In general, such a phosphorylase catalyzes the following reaction: glucose-comprising disaccharide, oligosaccharide, or polysaccharide+free phosphate e alpha-glucose-1-phosphate (alpha-G1P)+saccharide acceptor. A mammal-digestible saccharide, which the first phosphorylase uses as a substrate to produce alpha-GIP, typically comprises a disaccharide, oligosaccharide, or polysaccharide that has one or more glucose residues; such one or more glucose residues are used by the first phosphorylase to make alpha-G1P. Examples of a first phosphorylase herein include starch phosphorylase (EC 2.4.1.1) and sucrose phosphorylase (EC 2.4.1.7), which use starch and sucrose, respectively, to produce alpha-G1P. A second phosphorylase herein uses alpha-G1P (produced by the first phosphorylase) and a saccharide acceptor to produce an oligosaccharide or polysaccharide that is indigestible by a mammal (e.g., human). An example of such an indigestible saccharide is beta-glucan (e.g., an oligosaccharide or polysaccharide that comprises at least about 90%, 95%, or 100% beta-glycosidic linkages). In some aspects, a saccharide acceptor herein comprises beta-1,4 glycosidic linkages and/or the second phosphorylase enzyme is a cellodextrin phosphorylase that produces beta-1,4-glucan (e.g., comprising at least about 90%, 95%, or 100% beta-1,4 linkages). In some aspects, a saccharide acceptor comprises beta-1,3 glycosidic linkages, and the second phosphorylase enzyme is a beta-1,3-glucan phosphorylase that produces beta-1,3-glucan (e.g., comprising at least about 90%, 95%, or 100% beta-1,3 linkages). In some aspects, a food product or food precursor product is treated with first and second phosphorylase enzymes simultaneously, or in a step-wise manner beginning with the first phosphorylase enzyme.

In accordance with another aspect of the present invention, it was discovered that treating sucrose containing dairy containing products with glucosyltransferase converts sucrose to polyglucose and fructose. This results in a lowering of the weight concentration of the overall sugar molecules (sucrose, fructose, glucose, etc.) in the final dairy product. The generated polyglucose can be a be alpha or beta glucose linkage connecting carbons 1 to 6 in the glucose ring. Specifically, for alpha (1-3) glucan, above a degree of polymerization of 5, it becomes insoluble and can be used as a dietary fiber for health benefits.

In another aspect of the present invention, a method is presented of reducing the caloric content of, and/or increasing the dietary fiber content of a food product or food precursor product, the method having the steps of: treating a sucrose-containing food product or food precursor product with a glucosyltransferase under suitable conditions to convert sucrose of the food product or food precursor product to alpha-glucan, whereby the caloric content of the food product or food precursor product is reduced and/or the dietary fiber content of the food product or food precursor product is increased.

Preferably, the weight concentration of the sucrose in the food product or food precursor product after the treating step is between 0-80% of the weight concentration of the sucrose of the food product or food precursor product that existed before the treating step. More preferably, the weight concentration of the sucrose in the food product or food precursor product after said treating step is between 0-30% of the weight concentration of the sucrose of the food product or food precursor product that existed before the treating step.

Preferably, the alpha-glucan has a DPw of 5-5000.

Preferably, the alpha-glucan is alpha-1,3-glucan.

More preferably, the alpha-1,3-glucan has at least 50% alpha-1,3 linkages and a DPw of 5-1600.

Preferably, the food product or food precursor product comprises a dairy ingredient.

Preferred glycosyl transferases for this aspect of the present invention are as set forth above.

The present disclosure is described in further detail in the following examples, which are not in any way intended to limit the scope of the disclosure as claimed. The attached figures are meant to be considered as integral parts of the specification and description of the disclosure. The following examples are offered to illustrate, but not to limit the claimed disclosure.

### EXAMPLES

### Example 1 GTFJ

GTFJ is a glucosyl transferase enzyme derived from Streptococcus salivarius SK126 having the amino acid sequence as set forth in SEQ ID NO: 1. GTFJ was produced recombinantly in Bacillus subtilis.

### Example 2 GTF300

GTF300 has the following backbone substitutions relative to GTFJ. A510D:F607Y:R741S:D948G. The amino acid sequence of GTF300 is set forth in SEQ ID NO: 2. GTF300 was also produced in recombinantly in B. subtilis.

### Example 3: Standard Yogurt Procedure

Pre-pasteurized (72° C. for 15 s) bulk blended skimmed milk (0.1% fat) (Arla Foods, Denmark) stored at 4-6° C. was standardized to a desired protein (% w/w), fat (% w/w) and sucrose (% w/w) content by addition of skimmed milk powder (33% protein, 1.2% fat, 54% carbohydrate) from BBA Lactalis (Laval, Mayenne, France), cream (38% fat) from ArIa Foods Denmark), and sucrose (Granulated Sugar 500, Nordic Sugar A/S, Denmark). The standardized milk was then pasteurized and homogenized in a standard plate heat exchange pasteurizer. Homogenization was performed at 65° C. at 200 bar and pasteurization at 95° C. for 6 minutes, and then milk was cooled to 43° C. The milk was inoculated with a thermophilic starter culture at an inoculation rate of 20 DCU/100 L. Fermentation was followed using the CINAC multichannel pH system (Ysebaert, Frepillon, France), which monitored the pH development every 5 min. Fermentation was conducted until pH 4.60 and the product was cooled on a yogurt plate heat exchanger (SPX Flow Technology, Sussex, UK) and YTRON-ZP shear-pump system (YTRON Process Technology, Bad Endorf, Germany) to 24° C. The resulting stirred style yogurts were stored at 4-6° C. for further viscosity measurements.

### Example 4: Method for Measuring Apparent Viscosity

A rotational rheological test was employed to evaluate the viscosity of the stirred style yogurts. Flow curves were obtained with an Anton Paar MCR302 rheometer (Anton Paar GmbH, Ostfildern, Germany) using the cone plate measurement system. The test method was a controlled shear rate test (CSR), where the shear rate is controlled and the resulting shear stress is measured. The shear rate intervals applied to the samples were 0.1-200 s⁻¹, which defines the up-curve, and the reverse operation explains the down-curve (200-0.1 s⁻¹). The value of the measuring point duration was selected to be at least as long as the value of the reciprocal shear rate, which is valid for the up-curve. The tests were performed under constant temperature of 10° C., and each sample was analyzed in duplicates. A water bath was connected to the rheometer to ensure isothermal conditions.

From the flow curves the apparent viscosity was assessed, which is appropriate for fluids where the ratio of shear stress to shear rate varies with the shear rate. The apparent viscosity was extracted at either shear rate 10 Hz or 200 Hz. The apparent viscosity extracted at shear rate 10 Hz indicates the “thickness” of the sample. The apparent viscosity extracted at shear rate 200 s⁻¹ (200 Hz) is correlated to the sensory perception of “mouthfeel”.

### Example 5: GTF300 Addition at the Inoculation Step

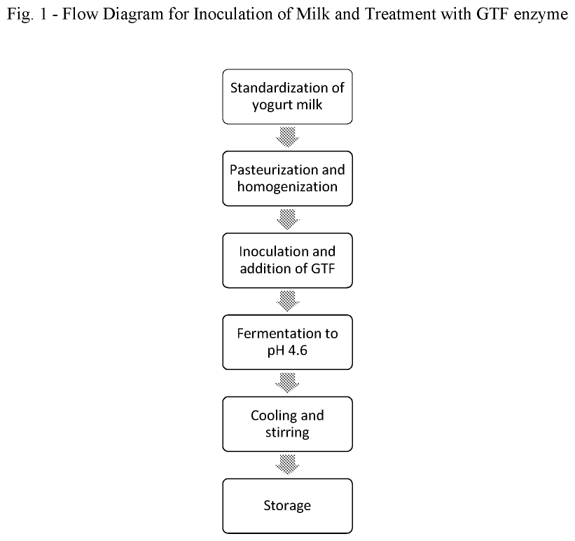

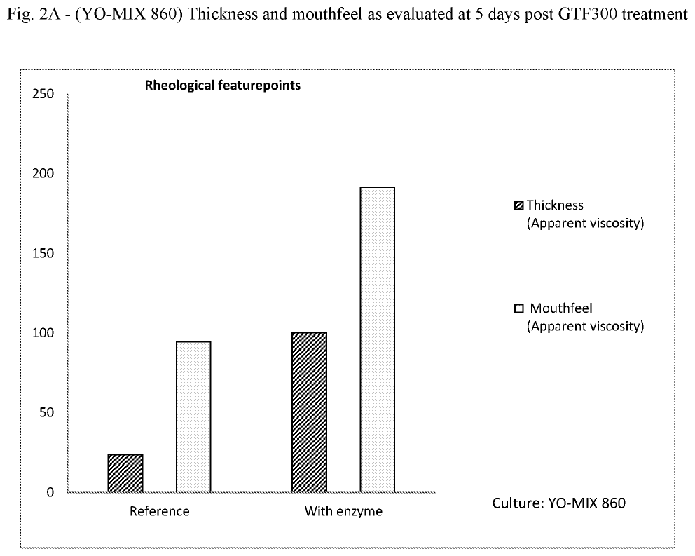

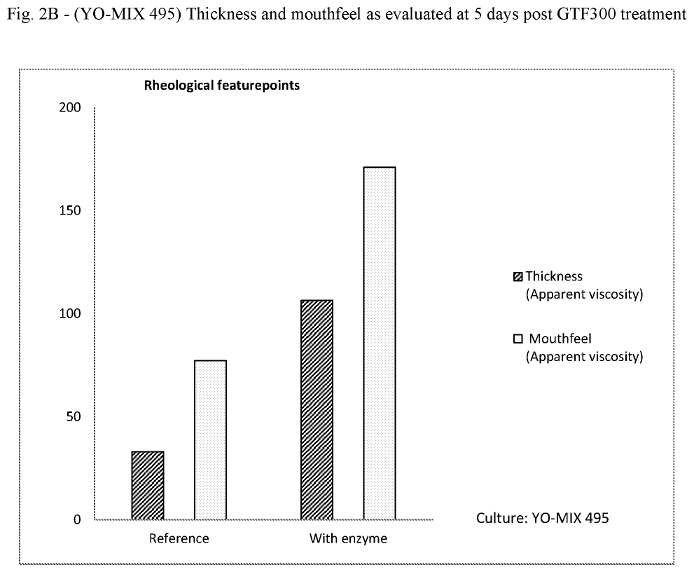

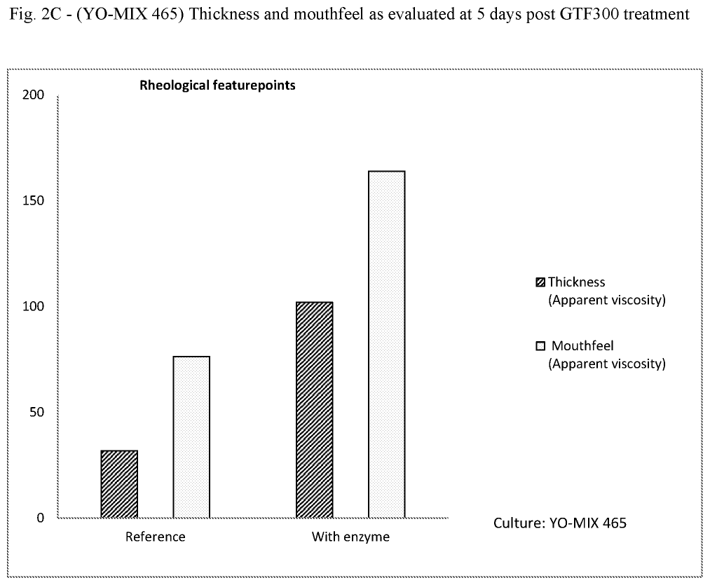

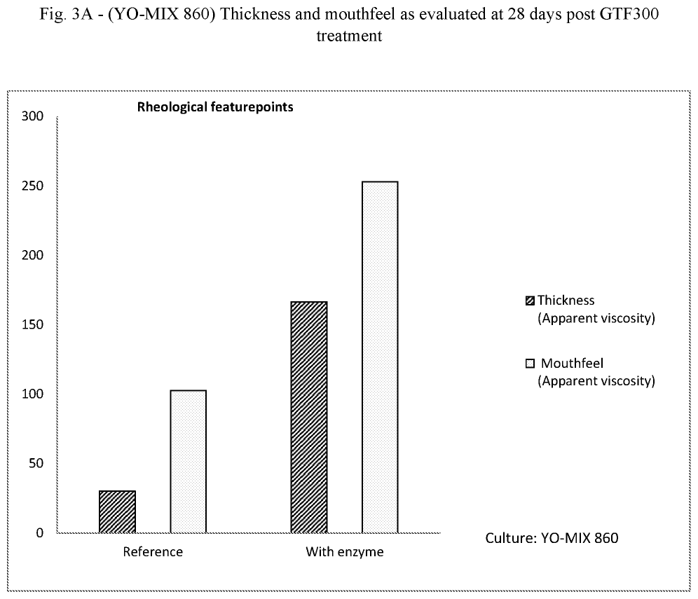

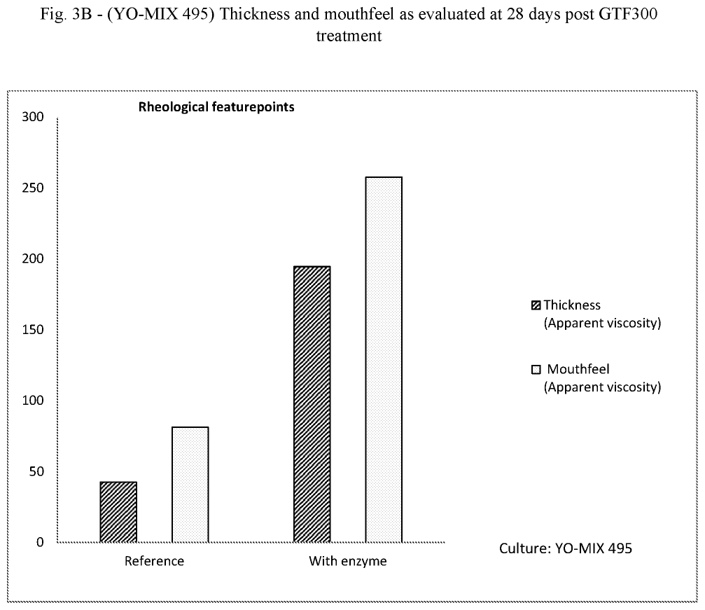

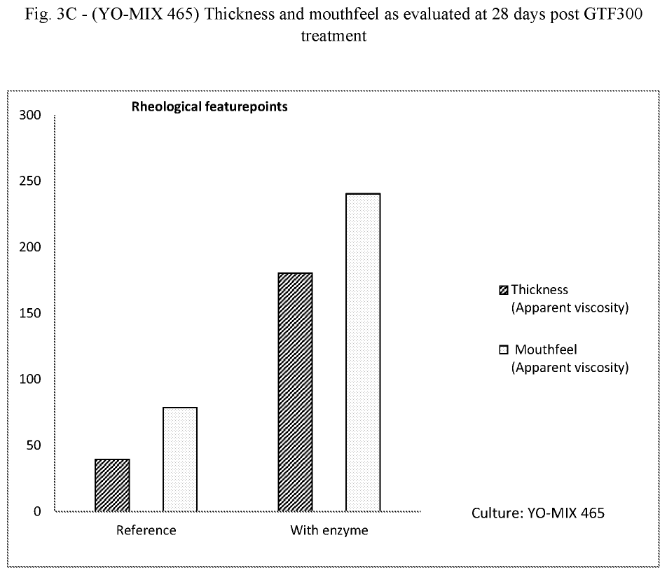

The texturing effect of GTF300 was investigated in a 4-liter scale set-up yogurt production. Fresh milk was standardized to 4.0% (w/w) protein and 1.0% (w/w) fat, 8.0% (w/w) sucrose, which was homogenized and pasteurized as described in example 3. GTF300 [2.5 mg/100 g of milk] was added at the inoculation step as schematically presented in FIG. 1. YO-MIX 860, YO-MIX 495, and YO-MIX 465, respectively, were employed as starter cultures (available from DuPont). After 5 and 28 days, respectively, of storage at 5°C. the texturing effect of GTF300 was assessed by rotational rheological test as described in example 4. The impact on viscosity of the non-enzymated and GTF300 added yogurt samples for the three different starter culture fermentations are presented in FIG. 2A to 2C (5 days) and FIG. 3A to 3C (28 days). The addition of GTF300 provided enhanced shear stress values over the entire shear range for all three starter cultures investigated. The addition of 2.5 mg GTF300 per 100 ml of milk resulted in an increased apparent viscosity (\dot{γ}=200 Hz) compared to the control by 103%, 122%, 116% for YM 860, YM 495, and YM 465, respectively, at day 5. The increase in texture was maintained at day 28 and, in fact, increased.

It was determined that GTF300 provides additional texture to that created by the gel network formed by addition of the starter cultures during acidification to pH 4.6. Moreover, the GTF300 provided texture survives the mechanical shear stresses caused by stirring, pumping and cooling of the fermented milk and this texture increase is maintained after 5 days of storage and, moreover, is maintained throughout the shelf life of the yogurt.

### Example 6: GTF300 Addition Prior to Heat-Treatment and Homogenization

The texturing effect of GTF300 was apparent when added at the inoculation step as shown in example 5. It was of interest to investigate whether the addition of GTF300 and the subsequent texture development could be established prior to pasteurization and homogenization of the base milk and maintained after such processing.

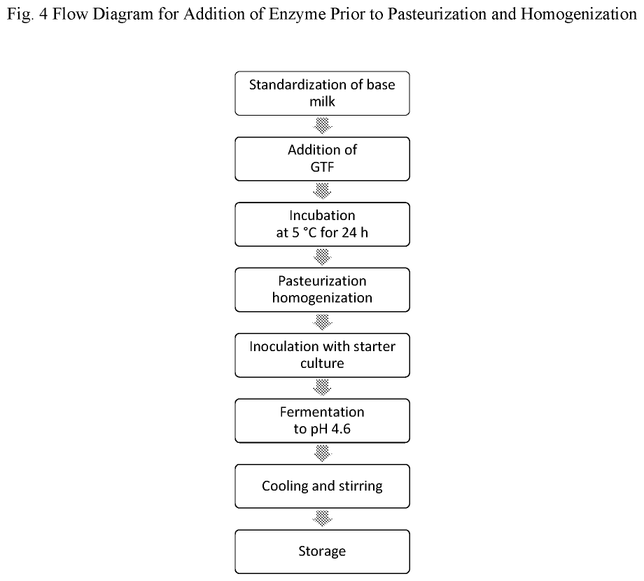

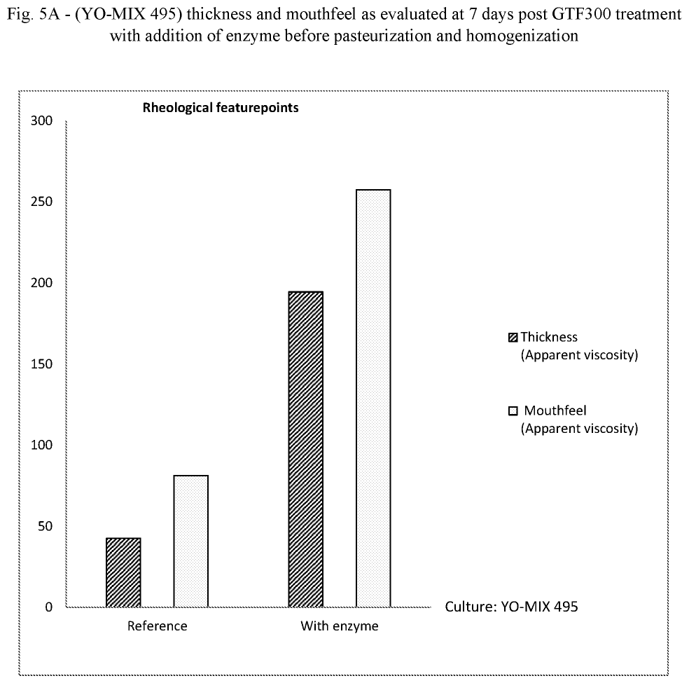

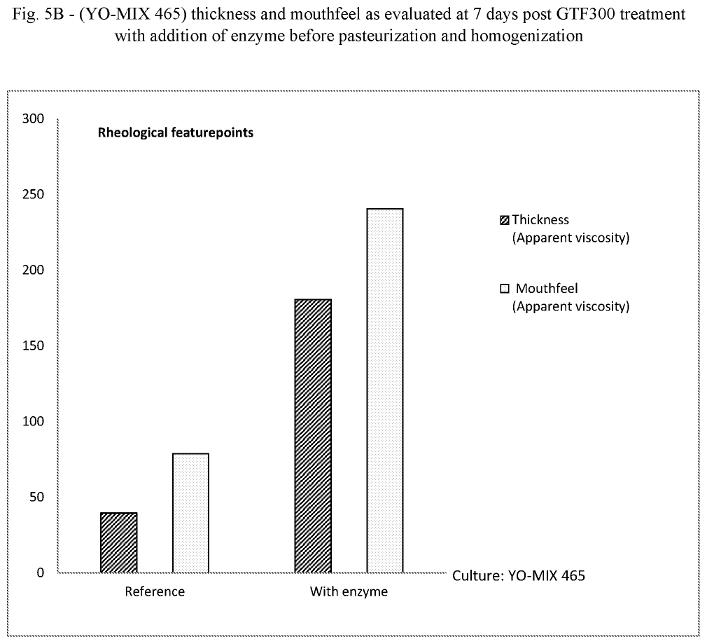

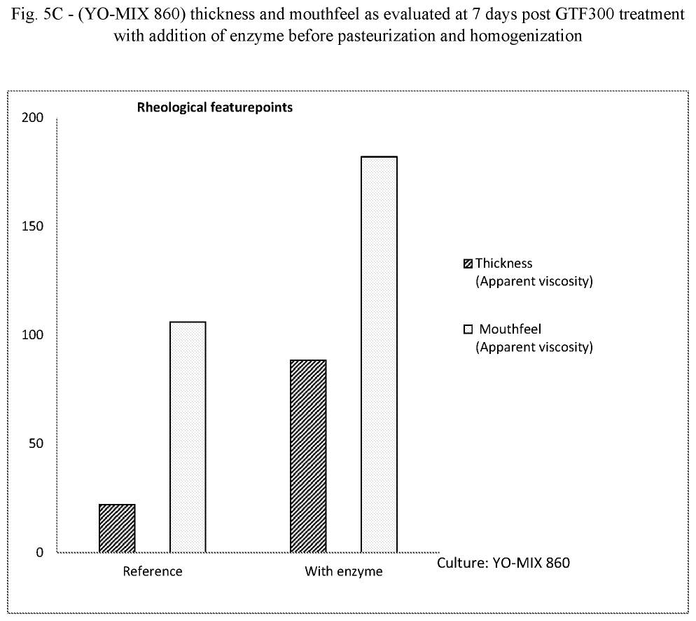

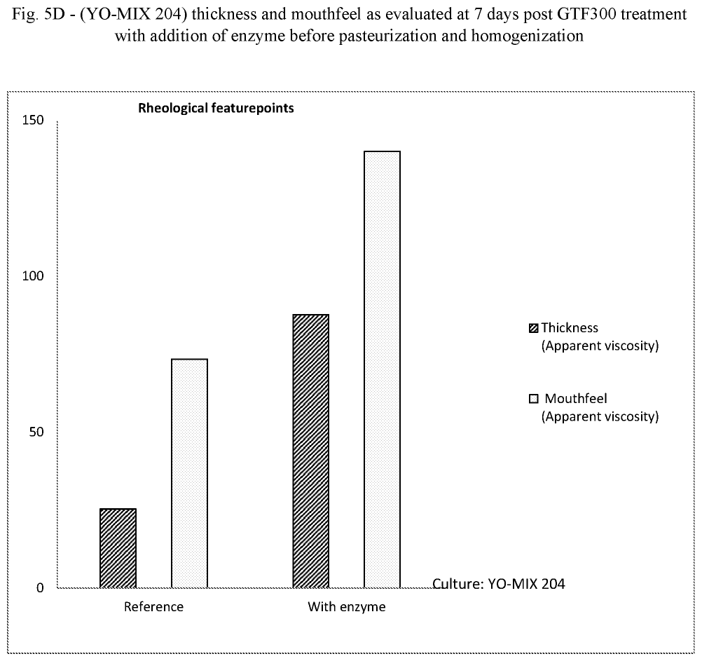

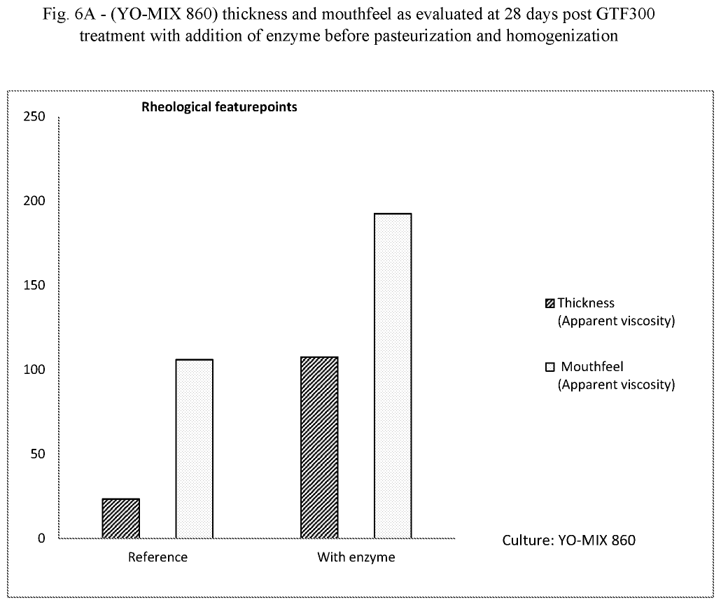

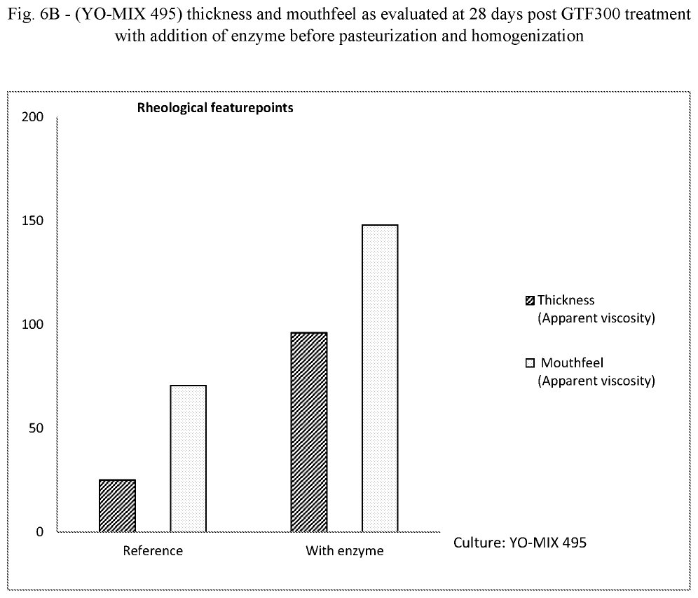

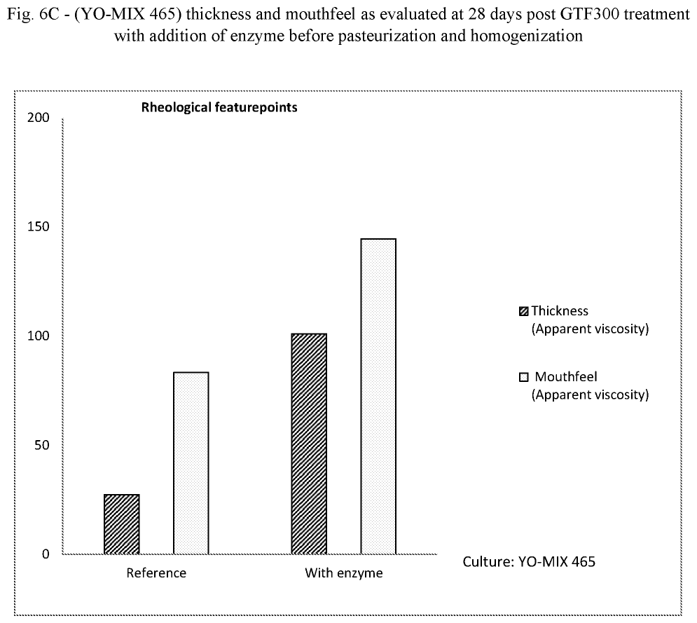

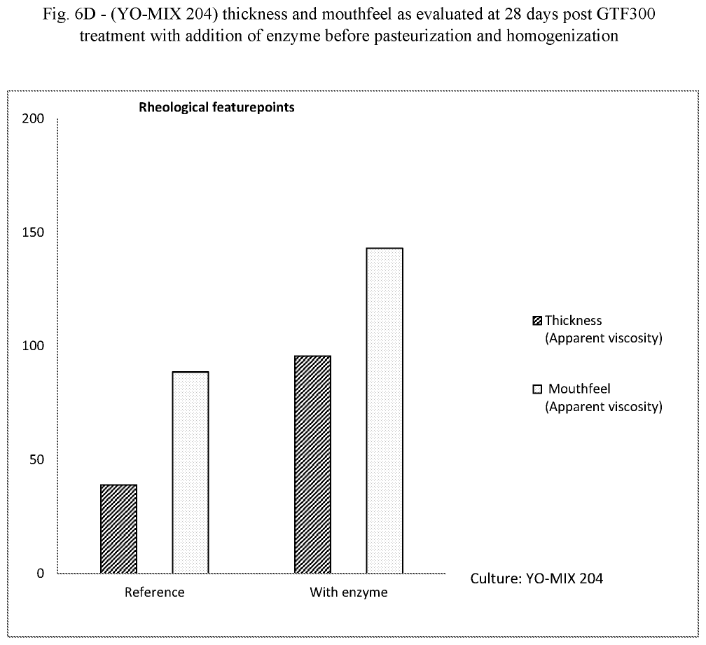

Therefore, GTF300 enzyme [3.75 mg per 100 ml of milk] was added to the base milk containing 8% (w/w %) sucrose, followed by an incubation step at 5° C. for 24 hours. Subsequently, the pasteurization and homogenization were performed as described in example 3. The production flow is schematically presented in FIG. 4. YO-MIX 860, YO-MIX 495, YO-MIX 465, and YO-MIX 204, respectively, were employed as starter cultures. After 7 and 28 days of shelf life the apparent viscosity was assessed as described in example 4. The resulting flow curves of the non-enzymated and GTF300 treated samples are shown in FIG. 5A-D (7 days) and FIG. 6A-D (28 days). The addition of GTF300 increased the apparent viscosity (\dot{γ}=200 Hz) by 72%, 47%, 62%, and 51% for YO-MIX 860, YO-MIX 495, YO-MIX 465 and YO-MIX 204, respectively, at day 7. This increase in texture was maintained at day 28, see FIG. 6A-D (28 days).

Surprisingly, it was observed that the texturing effect established by GTF300 could resist the mechanical shear of homogenization and pasteurization processes. The texture formed during the incubation step is, thus, able to withstand the before mentioned processing steps and in addition the shear from the cooling process at the end of fermentation.

### Example 7: GTF300 Addition to 2% and 4% Sucrose Yogurts

In example 6 the texturing effect of GTF300 was investigated for yogurts with a content of 8% sucrose. This prompted investigation of the texturing effect of GTF300 in yogurts with lower sucrose contents. Therefore, the performance of GTF300 was investigated for yogurts with 2% and 4% sucrose.

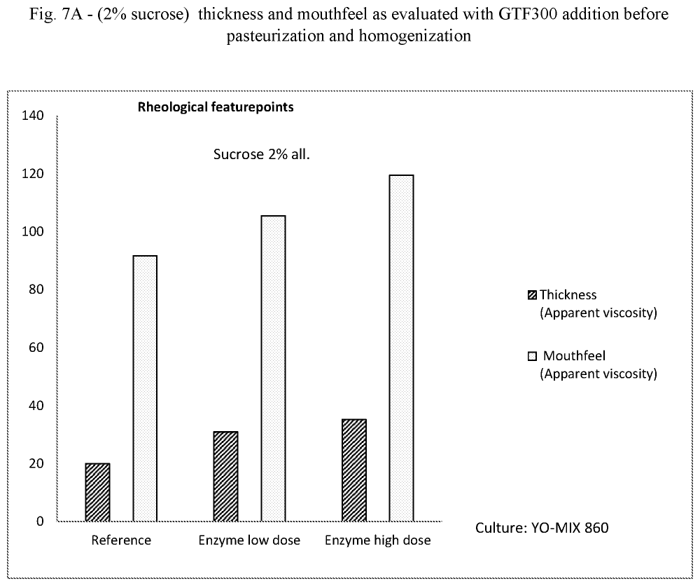

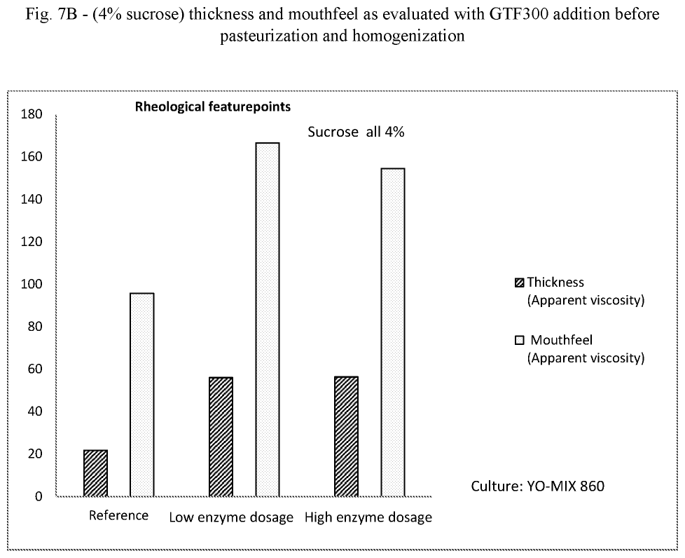

The milk was standardized to 4% (w/w) protein, 1% (w/w) fat, and 2% or 4% (w/w) sucrose, respectively, and pasteurized and homogenized as described in example 3. The dose of GTF300 was the same with regard to the sucrose content as in example 6. Additionally, doubling the dosage was also investigated. The addition of GTF300 was added to the milk followed by an incubation step at 5° C. for 24 hours prior to pasteurization and homogenization as outlined in FIG. 4. The texturing performance of GTF300 was investigated as described in example 4, and the results for day 7 are presented in FIG. 7A to 7B.

The texturing effect of GTF300 was apparent for both sucrose contents at 2% and 4%. The addition of GTF300 increased the apparent viscosity (\dot{γ}=200 Hz) by 74% and 61% when added at 1.88% sucrose and 3.76% sucrose for yogurts with 4% sucrose. For yogurts with 2% sucrose GTF300 increased the apparent viscosity (\dot{γ}=200 Hz) by 15% and 30% when added at 1.88% sucrose and 3.76% sucrose, respectively.

Even at reduced sucrose levels a substantial increase in texture can be enabled by the addition of GTF300.

### Example 8: Cooling of GTF300 Yogurt to 5° C. Instead of 24° C.

In the yogurt industry, the cooling of stirred type yogurt containing stabilizers such as starch is performed in a two-phase way. First, the fermented milk is stirred gently to obtain a homogenous matrix, and then cooled to typically between 20-24° C. Yogurt cups are then filled and kept at cold storage over a period of 10-12 hours to be cooled below 8° C. Filling the yogurt cups with yogurt at a temperature between 20-24° C. and then cooling is crucial to maintain the texture added by the starch. In this regard, cooling the yogurt to 8° C. and then filling, particularly if the cooling takes place under shear from pumps and plate heat exchanger, could result in a weak yogurt gel. Moreover, whey separation could occur during storage. Therefore, it was of interest to test if the texture formed by GTF300 in the fermented milk could resist cooling to 5° C. and possible shear during cooling and filling.

The milk was standardized to 4% (w/w) protein, 2% (w/w) fat, and 8% (w/w) sucrose and pasteurized and homogenized as described in example 3. The addition of GTF300 [3.75 mg per 100 ml of milk] was added at the inoculation step as schematically presented in FIG. 1. The texturing effect of GTF300 in fermented milk cooled to, respectively, 5° C. and 24° C., when filled in the cups, was assessed after 7 days as described in example 4.

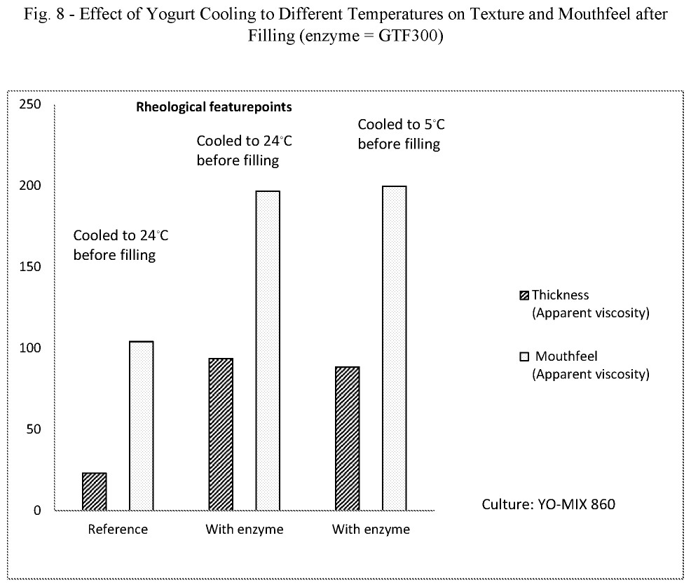

The addition of GTF300 enhanced the apparent viscosity (\dot{γ}=200 Hz) by 89% and 92% when cooled at 24° C. and 5° C., respectively, compared to a non-enzymated yogurt sample cooled at 24° C. The texture supplied by GTF300 is not sensitive to cooling at 5° C., and provides the same texture seen for GTF300 yogurts filled at 24° C. (see FIG. 8).

### Example 9: Addition of GTF300 to a Water Model System

The effect of GTF300 was pursued in a water model system with lactose (Variolac® 992 BG100, Arla Foods, Denmark) and/or sucrose (Granulated Sugar 500, Nordic Sugar A/S, Denmark) added. The sucrose and lactose contents were dissolved in the water by stirring the sample on a magnetic stirrer. The samples were kept at 5° C. until analysis of viscosity.

After 24 hours at 5° C. the viscosity was assessed by measuring the Brookfield viscosity (spindle S62, 30 rpm, 30 seconds).

TABLE 1
Brookfield viscosity of model systems with GTF300
at a dose of 2.5 mg per 100 ml of
milk. Brookfield viscosity (spindle S61 or spindle S62,
30 rpm, 30 seconds) was assessed after
24 hours at 5° C.

| Sample | Brookfield viscosity (cP) |
|---|---|
| Sample 1: | 2 cP |
| 5% lactose + GTF300 | |
| 2.5 mg/100 ml of milk | |
| Sample 2: | 200 cP |
| 5% lactose + 8% sucrose + | |
| GTF300 2.5 mg/100 | |
| ml of milk | |
| Sample 3: | 70 cP |
| 8% sucrose + GTF300 | |
| 2.5 mg/100 ml of milk | |

As shown above, with no sucrose GTF300 is unable to make polymer. As expected, glucan polymer is formed by the inclusion of 8% sucrose in the aqueous media. Surprisingly, however, it was determined that formation of glucan was substantially increased in the presence of lactose.

### Example 10: GTFJ Addition at the Inoculation Step

The texturing effect of GTFJ was investigated in a 4-liter scale set-up yogurt production. Fresh milk and cream was standardized to 4.0% (w/w) protein and 1.0% (w/w) fat, 8% (w/w) sucrose, homogenized and pasteurized as described in example 3. GTFJ was added at the inoculation step in several dosages (v/w %) [0.33 mg per 100 ml milk, 0.66 mg per 100 ml milk, 0.98 mg per 100 ml milk, 1.31 mg per 100 ml milk].

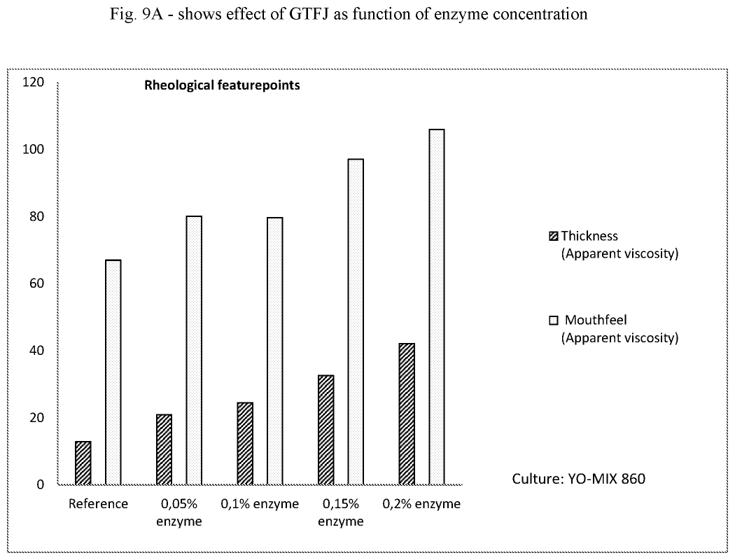

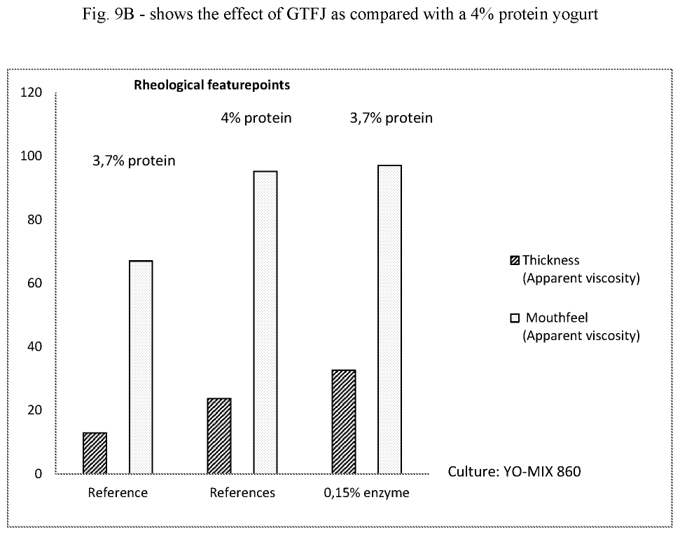

The employed starter culture was YO-MIX 860. After 7 days of storage the texturing effect of GTFJ was assessed by rotational rheological test as described in example 4. The results of the non-enzymated and GTFJ added yogurt samples for day 7 are presented in FIG. 9A. The addition of GTFJ enhanced the thickness for all applied dosages. The addition of 0.33 mg per 100 ml milk (0.05% enzyme), 0.66 mg per 100 ml milk (0.1% enzyme), 0.98 mg per 100 ml milk (0.15% enzyme), 1.31 mg per 100 mlmilk. (0.2% enzyme) increased the thickness by 62%, 92%, 154%, and 223%, respectively. The texturing effect of GTFJ was compared to the texturing effect of protein in FIG. 9B. It was seen that an addition of GTFJ [0.98 mg per 100 ml milk/0.15% enzyme] to a 3.7% protein yogurt increased the shear stress over the entire shear rate range. The flow curve of the 3.7% protein yogurt sample added GTFJ [0.98 mg per 100 ml milk] was compared to a non-enzymated 4.0% protein yogurt sample, and it was seen that the addition of GTFJ to a 3.7% protein yogurt sample could mimic the flow curve of the 4.0% protein yogurt sample. The apparent viscosity (\dot{γ}=200 Hz) of the 3.7% non-enzymated yogurt sample was 67 Pa. The addition of GTFJ increased the apparent viscosity (\dot{γ}=200 Hz) by 45% to 97 Pa. The apparent viscosity (\dot{γ}=200 Hz) of the 4.0%/0 non-enzymated yogurt sample was 95 Pa.

### Example 11: Lactose Incorporation in Polymers and Affect on Rate of Polymer Formation Materials and Methods

Sample Preparation and Photometric Measurement

The effect of lactose on GTF300 was investigated in a water model system with lactose (Variolac® 992 BG100, Arla Foods, Denmark) and/or sucrose (Granulated Sugar 500, Nordic Sugar A/S, Denmark) added. The sucrose and/or lactose contents (5% each) were dissolved in 100 mL water by stirring the sample on a magnetic stirrer. After all sugars were dissolved, 0.2% GTF300 was added to the samples. The sample were mixed for further 30 s and incubated without stirring or mixing at 25° C. for up to 48 h. Additionally, a milk sample (UHT-milk, 1.5% fat, Arla Foods, Denmark) with 5% sucrose in the milk was prepared equally to the samples with water. All samples were adjusted to a pH of 6.7 with acetic acid, if necessary.

After 24 and 48 h incubation, 250 μL of the samples containing water were transferred to a microtitter plate and the change in adsorption compared to samples without enzyme addition was measured photometrically (Multiskan™ FC Microplate Photometer, ThermoFischer Scientific, United States) at 340 nm.

Measurement of Soluble Sugars

All samples (water samples and milk samples) were analyzed regarding their composition of soluble sugars (lactose and sucrose).

Quantification of the sugars was performed by HPLC. Prior to HPLC analysis, the samples were diluted 10-fold in water and centrifuged at 13.000 rpm for 10 min. Subsequently, 475 μL of the supernatant were mixed with 25 μL of 20% ribose in water. Ribose acted as an internal standard during the quantification and analysis. The so prepared samples were mixed with 25 μL of Carrez reagent (15 g of potassium hexacyanoferrate(II) trihydrate in 100 mL water) and 25 μL Carrez II (30 g of zinc sulphate heptahydrate in 100 mL water). The samples were mixed and subsequently centrifuged at 13.000 rpm for 10 min. Afterward, 280 μL of the supernatant were filtered through a 0.22 μm filterplate and used for injection to the HPLC.

HPLC analysis was carried out on a Dionex Ultimate 3000 HPLC System (Thermo Fischer Scientific) equipped with a DGP-3600SD Dual Gradient analytical pump, WPS-3000TSL thermostat autosampler, TCC-3000SD thermostated column oven, and a RI-101 refractive index detector (Shodex, JM Science). Chromeleon datasystem software (Version 7.2) was used for data acquisition and analysis. The injection volume for each sample was set to 10 μL. Samples were held at 20° C. in the thermostated autosampler compartment. Chromatography was performed with a RSO oligosaccharide 200×10 mm column, Ag⁺4% crosslinked (Phenomenex, The Netherlands) equipped with a guard column (RSO oligosaccharide 60×10 mm column, Ag⁺4% crosslinked, Phenomenex, The Netherlands) at 70° C. The column was eluted with double distilled water at a flow rate of 0.29 mL/min. An isocratic flow of 0.29 mL/min was maintained throughout analysis with a total run time of 65 min. The eluent was monitored by means of refraction index detector (RI-101, Shodex, JM Science) and quantification was made by the peak area relative to the peak area of the given standard. The sugars to be quantified were used as a standard for quantification.

Results

TABLE 2
Absorption increase at 340 nm of model systems
with GTF300 at a dose of 0.2% in water.

| Sample | 24 h incubation | 48 h incubation |
|---|---|---|
| 5% sucrose in water | 1.084 ± 0.004 | 2.001 ± 0.005 |
| 5% lactose in water | 0.030 ± 0.000 | 0.030 ± 0.000 |
| 5% sucrose + 5% | 1.776 ± 0.047 | 1.896 ± 0.101 |
| lactose in water | | |

As shown above, after 24 h the absorption in the sample containing lactose and sucrose was increased by 64% compared to the sample containing lactose only. In contrast, no increase in absorption was detected with lactose as the only sugar present. Therefore, lactose alone was not converted by the enzyme. However, lactose seemed to act as an acceptor for the glycosyl-enzyme complex, leading to a faster conversion/polymer formation. However, after 48 h the absorption in the sample containing sucrose only and of the sample containing sucrose and lactose was comparable.

TABLE 3
Sucrose and lactose concentration in the supernatant of
model systems with GTF300 at a dose of
0.2% in water or milk after varied incubation times
at 25° C.

| Sample | Sucrose | Lactose |
|---|---|---|
| 5% sucrose in water | | |
| 0 h | 4.99% | 0.00% |
| 24 h | 7.59% | 0.00% |
| 48 h | 0.79% | 0.00% |
| 5% lactose in water | | |
| 0 h | 0.00% | 4.98% |
| 24 h | 0.00% | 4.98% |
| 48 h | 0.00% | 4.97% |
| 5% sucrose + 5% lactose in | | |
| water | | |
| 0 h | 5.14% | 5.02% |
| 24 h | 2.04% | 4.47% |
| 48 h | 0.00% | 4.10% |
| 5% sucrose milk | | |
| 0 h | 5.00% | 4.93% |
| 24 h | 0.20% | 3.73% |
| 48 h | 0.00% | 3.68% |

As shown above, the lactose content was stable in the sample containing only 5% lactose and GTF300. Lactose as the only sugar present was not converted by the enzyme and no polymers or single sugars were formed. In contrast, sucrose in water was converted by GTF300. After 24 h and 48 h a remaining concentration of 52% and 16% of the initial sucrose level, was detected respectively. If sucrose and lactose were present simultaneously in the water sample, the sucrose level decreased to 40% of its initial value after 24 h and no sucrose was detected in the sample after 48 h. At same times, the lactose level in the supernatant decreased by 11% and 18% after 24 h and 48 h respectively. No additional glucose or galactose, which would indicate a lactose hydrolysis were detected. Consequently, lactose was able to act as an acceptor for the glycosyl-enzyme complex. Unsoluble sugar polymers containing lactose were formed, and the lactose concentration in the supernatant decreased. Surprisingly, in milk this effect increased even further compared to a sample with lactose and sucrose in water. Here, a remaining sucrose concentration of 4% and 0% of its original value was detected after 24 h and 48 h respectively. The lactose concentration decreased by 24% after 24 h and 25% after 48 h. Therefore, a milk base promoted the lactose incorporation further.

In corporation of lactose in the polymers formed was also visible in the chromatograms from the HPLC (see FIG. 10). In general, higher amounts of soluble polymers (DP3-DP7) were formed in the presence of lactose compared to sucrose without lactose. Furthermore, when lactose was present, the soluble polymers eluted later from the column (up to 30 s) and the peaks were not symmetrical like for sucrose only, but showed a fronting and/or a shoulder. Therefore, more than one type of sugar polymer with equal degrees of polymerization eluted at the similar times from the column.

Although the foregoing invention has been described in some detail by way of illustration and example, for purposes of clarity of understanding, certain changes and modifications can be practiced within the scope of the appended claims. In addition, each reference provided herein is incorporated by reference in its entirety for all purposes to the same extent as if each reference was individually incorporated by reference. To the extent the content of any citation, including website or accession number may change with time, the version in effect at the filing date of this application is meant. Unless otherwise apparent from the context any step, element, aspect, feature of embodiment can be used in combination with any other.

What is claimed is:

1. A method of making a yogurt product having improved texture, wherein said improved texture comprises increased thickness and/or increased mouthfeel, said method comprising the steps of:
providing milk;
adding sucrose to the milk to form sweetened milk;
contacting said sweetened milk with a glucosyl transferase to form an insoluble glucose polymer;
inoculating with a starter culture;
and fermenting to provide the yogurt product having improved texture comprising increased thickness and/or increased mouthfeel.

2. A method according to claim 1 wherein the milk is cow's milk.

3. A method according to claim 2 wherein the milk is selected from the group consisting of raw milk, pre-pasteurized milk, whole milk, skim milk, reconstituted milk, lactase treated milk, reduced lactose milk, lactose free milk and condensed milk.

4. A method according to claim 3 wherein the milk is raw milk.

5. A method according to any of the preceding claims comprising the additional steps of homogenizing and pasteurizing the milk.

6. A method according to claim 5 wherein the step of contacting with glucosyl transferase is performed after the steps of homogenizing and pasteurizing.

7. A method according to claim 5 wherein the step of contacting with glucosyl transferase is performed before the steps of homogenizing and pasteurizing.

8. A method according to any of the preceding claims wherein the sucrose is added to constitute about 0.1 to 12% (w/w).

9. A method according to claim 8 wherein the sucrose is added to constitute about 2 to 8% (w/w).

10. A method according to claim 9 wherein the sucrose is added to constitute about 4 to 6% (w/w).

11. A method according to any of the preceding claims wherein the glucosyl transferase comprises an enzyme which has at least 70% sequence identity to and enzyme selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO: 12), GTF2919 (SEQ ID NO: 13), GTF2678 (SEQ ID NO; 14), and GTF3929 (SEQ ID NO: 15).

12. A method according to claim 11 wherein the glucosyl transferase comprises an enzyme which has at least 80% sequence identity to an enzyme selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO: 12), GTF2919 (SEQ ID NO: 13), GTF2678 (SEQ ID NO; 14), and GTF3929 (SEQ ID NO: 15).

13. A method according to claim 12 wherein the glucosyl transferase comprises an enzyme which has at least 90% sequence identity to an enzyme selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO: 12), GTF2919 (SEQ ID NO: 13), GTF2678 (SEQ ID NO; 14), and GTF3929 (SEQ ID NO: 15).

14. A method according to claim 13 wherein the glucosyl transferase comprises an enzyme which has at least 95% sequence identity to an enzyme selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO: 12), GTF2919 (SEQ ID NO: 13), GTF2678 (SEQ ID NO; 14), and GTF3929 (SEQ ID NO: 15).

15. A method according to claim 14 wherein the glucosyl transferase is selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO. 12), GTF2919 (SEQ ID NO: 13), GTF2678 (SEQ ID NO; 14), and GTF3929 (SEQ ID NO: 15).

16. A method according to any of claims 11-15 wherein the glucosyl transferase is present in the milk in an amount from about 0.005 mg per 100 ml milk to about 15 mg per 100 ml milk.

17. A method according to claim 16 wherein the glucosyltransferase is present in the milk in an amount from about 0.03 mg per 100 ml milk to about 12.5 mg per 100 ml milk.

18. A method according to claim 15 wherein the glucosyl transferase is GTFJ (SEQ ID NO: 1).

19. A method according to claim 18 wherein the GTFJ is present in an amount from about 0.033 mg per 100 ml milk to about 12.5 mg per 100 ml milk.

20. A method according to claim 19 wherein the GTFJ is present in an amount from about 0.3 mg per 100 ml milk to about 5.0 mg per 100 ml milk.

21. A method according to claim 15 wherein the glucosyl transferase is GTF300 (SEQ ID NO: 2).

22. A method according to claim 21 wherein the GTF300 is present in an amount from about 0.033 mg per 100 ml to about 12.5 mg per 100 ml milk.

23. A method according to claim 22 wherein the GTF300 is present in an amount from about 0.3 mg per 100 ml milk to about 5 mg per 100 ml milk.

24. A method according to any of the preceding claims wherein the increased texture comprises increased thickness.

25. A method according to claim 24 wherein the thickness is increased by 30% or more.

26. A method according to claim 25 wherein the thickness is increased by 50% or more.

27. A method according to claim 26 wherein the thickness is increased by 70% or more.

28. A method according to claim 27 wherein the thickness is increased by 90% or more.

29. A method according to claim 28 wherein the thickness is increased by 100% or more.

30. A method according to claim 29 wherein the thickness is increased by 110% or more.

31. A method according to claim 30 wherein the thickness is increased by 120% or more.

32. A method according to any of claims 1-23 wherein the increased texture comprises increased mouthfeel.

33. A method according to claim 32 wherein the mouthfeel is increased by 30% or more.

34. A method according to claim 33 wherein the mouthfeel is increased by 50% or more.

35. A method according to claim 34 wherein the mouthfeel is increased by 70% or more.

36. A method according to claim 35 wherein the mouthfeel is increased by 90% or more.

37. A method according to claim 36 wherein the mouthfeel is increased by 100% or more.

38. A method according to claim 37 wherein the mouthfeel is increased by 110% or more.

39. A method according to claim 38 wherein the mouthfeel is increased by 120% or more.

40. A method according to any of the preceding claims further comprising the steps of
cooling the yogurt of to a temperature of between 5 and 10° C. to provide a chilled yogurt, and
pouring the chilled yogurt into preformed containers.

41. A method according to claim 40 wherein the preformed containers provide a single serving of yogurt.

42. A method according to any of the preceding claims wherein the milk is non-fat milk to provide a non-fat yogurt.

43. A method according to any of the preceding claims wherein the protein content of the milk is adjusted to at least about 3% (w/w).

44. A method according to claim 43 wherein the protein content of the milk is adjusted to at least about 3.5% (w/w).

45. A method according to claim 44 wherein the protein content of the milk is adjusted to at least about 3.7% (w/w).

46. A method according to claim 45 wherein the protein content of the milk is adjusted to at least about 3.8% (w/w).

47. A method according to claim 46 wherein the protein content of the milk is adjusted to at least about 3.9% (w/w).

48. A method according to claim 47 wherein the protein content of the milk is adjusted to at least about 4.0% (w/w).

49. A yogurt produced in accordance with any of the preceding claims.

50. The yogurt of claim 49 further comprising pectin.

51. The method according to any of claims 1 to 48 wherein the milk comprises at least 4% lactose (w/w).

52. The method according to claim 51 wherein the milk comprises at least 4.5% lactose.

53. A method of making a reduced sugar food product having improved texture, said method comprising the steps of:
providing a food matrix comprising sucrose and lactose; and
contacting said food matrix with a glucosyl transferase to form an insoluble glucose polymer.

54. A method according to claim 53 wherein the sucrose is from about 0.1 to about 12% (w/w).

55. A method according to claim 54 wherein the sucrose is from about 2 to about 8% (w/w).

56. A method according to claim 55 wherein the sucrose is from about 4 to about 6% (w/w).

57. A method according to any of claims 53 to 56 wherein the lactose is from about 0.1 to about 12% (w/w).

58. A method according to claim 57 wherein the lactose is from about 2 to about 8% (w/w)

59. A method according to claim 58 wherein the lactose is from about 4 to about 6% (w/w).

60. A method according to any of claims 53 to 59 wherein the improved texture comprises increased thickness and/or increased mouthfeel.

61. A method according to any of claims 53 to 60 wherein the food matrix is a diary product, a beverage, a dough or bread, a confectionary, a fermented beverage, a dressing, a sauce, or a processed meat.

62. A method according to any of claims 53 to 61 wherein the glucosyl transferase comprises an enzyme which has at least 70% sequence identity to and enzyme selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO: 12), GTF2919 (SEQ ID NO: 13), GTF2678 (SEQ ID NO; 14), and GTF3929 (SEQ ID NO: 15).

63. A method according to claim 62 wherein the glucosyl transferase comprises an enzyme which has at least 80% sequence identity to an enzyme selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO: 12), GTF2919 (SEQ ID NO: 13), GTF2678 (SEQ ID NO; 14), and GTF3929 (SEQ ID NO: 15).

64. A method according to claim 63 wherein the glucosyl transferase comprises an enzyme which has at least 90% sequence identity to an enzyme selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO: 12), GTF2919 (SEQ ID NO: 13), GTF2678 (SEQ ID NO; 14), and GTF3929 (SEQ ID NO: 15).

65. A method according to claim 64 wherein the glucosyl transferase comprises an enzyme which has at least 95% sequence identity to an enzyme selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO: 12), GTF2919 (SEQ ID NO: 13), GTF2678 (SEQ ID NO; 14), and GTF3929 (SEQ ID NO: 15).

66. A method according to claim 65 wherein the glucosyl transferase is selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO. 12), GTF2919 (SEQ ID NO: 13), GTF2678 (SEQ ID NO; 14), and GTF3929 (SEQ ID NO: 15).

67. A method of reducing the caloric content of, and/or increasing the dietary fiber content of, a food product or food precursor product, said method comprising:
treating a saccharide-containing food product or food precursor product with first and second phosphorylase enzymes under suitable conditions, wherein the saccharide is mammal-digestible and comprises glucose, wherein the first phosphorylase enzyme converts the mammal-digestible saccharide to products including alpha-glucose-1-phosphate (alpha-G1P), and the second phosphorylase enzyme reacts the alpha-GIP with a saccharide acceptor to produce a mammal-indigestible saccharide, whereby the caloric content of the food product or food precursor product is reduced and/or the dietary fiber content of the food product or food precursor product is increased.

68. A method of claim 67, wherein the food product or food precursor product is treated with the first and second phosphorylase enzymes simultaneously, or in a step-wise manner beginning with the first phosphorylase enzyme.

69. A method of claim 67, wherein the mammal-digestible saccharide comprises a disaccharide, oligosaccharide, or polysaccharide.

70. A method of claim 67, wherein mammal-digestible saccharide comprises sucrose or starch, and wherein the first phosphorylase enzyme is, respectively, a sucrose phosphorylase or starch phosphorylase.

71. A method of claim 67, wherein the mammal-indigestible saccharide is a beta-glucan.

72. A method according to any of claims 67 to 71, wherein the saccharide acceptor comprises beta-1,4 glycosidic linkages and the second phosphorylase enzyme is a cellodextrin phosphorylase that produces beta-glucan comprising beta-1,4 glycosidic linkages using the saccharide acceptor as a substrate.

73. A method of claim 67 to 71, wherein the saccharide acceptor comprises beta-1,3 glycosidic linkages, and the second phosphorylase enzyme is a beta-1,3-glucan phosphorylase that produces beta-glucan comprising beta-1,3 glycosidic linkages using the saccharide acceptor as a substrate.

74. A method of reducing the caloric content of, and/or increasing the dietary fiber content of a food product or food precursor product, said method comprising: treating a sucrose-containing food product or food precursor product with a glucosyltransferase under suitable conditions to convert sucrose of the food product or food precursor product to alpha-glucan, whereby the caloric content of the food product or food precursor product is reduced and/or the dietary fiber content of the food product or food precursor product is increased.

75. A method according to claim 74, wherein the weight concentration of the sucrose in the food product or food precursor product after said treating step is between 0-80% of the weight concentration of the sucrose of the food product or food precursor product that existed before the treating step.

76. A method according to claim 75, wherein the weight concentration of the sucrose in the food product or food precursor product after said treating step is between 0-30% of the weight concentration of the sucrose of the food product or food precursor product that existed before the treating step.

77. A method according to claim 74, wherein the alpha-glucan has a DPw of 5-5000.

78. A method according to claim 74, wherein the alpha-glucan is alpha-1,3-glucan.

79. A method according to claim 78, wherein the alpha-1,3-glucan has at least 50% alpha-1,3 linkages and a DPw of 5-1600.

80. A method according to any of claims 74 to 79, wherein the food product or food precursor product comprises a dairy ingredient.

81. A method according to any of claims 75 to 89 wherein the glucosyl transferase comprises an enzyme which has at least 70% sequence identity to and enzyme selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO: 12), GTF2919 (SEQ ID NO: 13), GTF2678 (SEQ ID NO; 14), and GTF3929 (SEQ ID NO: 15).

82. A method according to claim 81 wherein the glucosyl transferase comprises an enzyme which has at least 80% sequence identity to an enzyme selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO: 12), GTF2919 (SEQ ID NO: 13), GTF2678 (SEQ ID NO; 14), and GTF3929 (SEQ ID NO: 15).

83. A method according to claim 82 wherein the glucosyl transferase comprises an enzyme which has at least 90% sequence identity to an enzyme selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO: 12), GTF2919 (SEQ ID NO: 13), GTF2678 (SEQ ID NO; 14), and GTF3929 (SEQ ID NO: 15).

84. A method according to claim 83 wherein the glucosyl transferase comprises an enzyme which has at least 95% sequence identity to an enzyme selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO: 12), GTF2919 (SEQ ID NO: 13), GTF2678 (SEQ ID NO; 14), and GTF3929 (SEQ ID NO: 15).

85. A method according to claim 84 wherein the glucosyl transferase is selected from the group consisting of GTFJ (SEQ ID NO: 1), GTF300 (SEQ ID NO: 2), GTF0874 (SEQ ID NO: 3), GTF6855 (SEQ ID NO: 4), GTF2379 (SEQ ID NO: 5), GTF7527 (SEQ ID NO: 6), GTF1724 (SEQ ID NO: 7), GTF0544 (SEQ ID NO: 8), GTF5926 (SEQ ID NO: 9), GTF4297 (SEQ ID NO: 10), GTF5618 (SEQ ID NO: 11), GTF2765 (SEQ ID NO: 12), GTF2919 (SEQ ID NO: 13), GTF2678 (SEQ ID NO; 14), and GTF3929 (SEQ ID NO: 15).
